(19) United States
(12) Patent Application Publication
(10) Pub. No.: US 2015/0247207 A1
(43) Pub. Date: Sep. 3, 2015

(54) MICROARRAY FOR THE DETECTION OF AN ANGIOSTATIC TUMOR STAGE OF COLORECTAL CARCINOMA

(71) Applicant: Friedrich-Alexander-Universitaet Erlangen, Erlangen (DE)

(72) Inventors: Roland S. Croner, Roettenbach (DE); Michael Stuerzl, Erlangen (DE); Elisabeth Naschberger, Erlangen (AT)

(21) Appl. No.: 14/712,325

(22) Filed: May 14, 2015

Related U.S. Application Data
(63) Continuation of application No. 12/516,475, filed on May 27, 2009.

(51) Int. Cl. C12Q 1/68; G01N 33/574

(52) U.S. Cl. CPC C12Q 1/6886; G01N 33/57419; C12Q 2600/16; C12Q 2600/118

(57) ABSTRACT

A microarray for the detection of an angiostatic tumor stage/tumor area of colorectal carcinoma in a patient is provided. In some embodiments, the microarray comprises gene probes capable of specifically hybridizing to predefined nucleic acids. Also provided are inhibitors or modulators of one or more of these nucleic acids, pharmaceutical compositions comprising the disclosed inhibitors and/or modulators, ex vivo methods for diagnosis of an angiostatic tumor stage/tumor area in a patient suffering from a colorectal carcinoma, and methods to predict the response of patients with colorectal carcinoma and other diseases to therapy.

### CROSS REFERENCE TO RELATED APPLICATIONS

The presently disclosed subject matter in a continuation of U.S. patent application Ser. No. 12/516,475, filed May 27, 2009, which itself is a National Stage entry of PCT International Patent Application Serial No. PCT/EP2007/06522, filed Nov. 19, 2007, which itself claims the benefit of U.S. Provisional Patent Application Ser. No. 60/861,624, filed Nov. 29, 2006, the disclosure of which is incorporated herein by reference in its entirety.

### TECHNICAL FIELD

The present invention is directed to a microarray for the detection of an angiostatic tumor stage/tumor area of colorectal carcinoma in a patient, wherein the microarray comprises gene probes capable of specifically hybridizing to predefined nucleic acids. The invention is further directed to an inhibitor or modulator of one or more of these nucleic acids, as well as to a pharmaceutical composition, comprising those inhibitors or modulators. In a further aspect, the present invention is directed to an ex vivo method for the diagnosis of an angiostatic tumor stage/tumor area in a patient suffering from a colorectal carcinoma. In a further aspect the invention is directed to predict the response of patients with colorectal carcinoma but also other diseases to therapy.

### BACKGROUND

Colorectal Cancer is the third most frequently occurring cancer in both sexes worldwide. It ranks second in developed countries (Hawk and Levin, 2005). The cumulative life time risk of developing colorectal cancer is about 6% (Smith et al., 2002). Despite the advances in the treatment of this disease the 5-year survival is only 62% (Smith et al., 2002).

Three pathways have been described as the basis for malignant transformation within the colon. These are the chromosomal instability pathway, the microsatellite instability pathway (Vogelstein et al., 1988) and the methylation pathway (Jass, 2002).

Malignant transformation of the colorectal epithelium typically occurs as a multistep process that requires cumulative damage to different genes within several cellular generations. Initially cryptal hyperplasia, a proliferation of normal-appearing cells, commonly results from genetic or epigenetic changes in pathways regulating cell cycle progression or apoptosis such as APC or Bcl-2 (Baylin and Herman, 2000). The transition from hyperproliferation to dysplasia is characterized by abnormal nuclear and/or cellular shapes in crypts with larger cells, often characterized by mutations in k-ras (Takayama et al., 2001). Progression from these aberrant crypt foci to adenoma, and subsequently to carcinoma, is typically associated with additional aberrations involving SMAD-2/4, DCC, and p53 (Ilyas et al., 1999). In addition to the genetic changes in the tumor cells two important stroma reactions are associated with colorectal cancer pathogenesis: angiogenesis and inflammation.

### Angiogenesis in Colorectal Carcinoma

Tumor growth beyond the critical two to three millimeter diameter and metastasis require angiogenesis. The important role of angiogenesis in colorectal cancer progression has been convincingly documented. It has been shown that microvessel density increases around primary tumors compared with normal mucosa or adenomas (Bossi et al., 1995), and is a strong independent predictor of poor outcome (Takebayashi et al., 1996). High microvessel density is associated with a greater than 3-fold risk of death from colorectal cancer (Choi et al., 1998). In addition, vascular endothelial growth factor (VEGF) expression is significantly increased in patients with all stages of colorectal carcinoma as compared to controls (Kumar et al., 1998). Intratumor expression of VEGF was found to be associated with a nearly 2-fold increase of death risk from colorectal cancer (Ishigami et al., 1998) and correlated with increasing tumor stage, decreased overall survival, and decreased disease-free survival (Kahlenberg et al., 2003; Kang et al., 1997). Recently, all of these observations were convincingly supported in a clinical study. In this study an anti-VEGF antibody (Bevacizumab, Avastin) was added to flourouracil-based combination chemotherapy. This approach resulted in statistically significant and clinically meaningful improvement in survival among patients with metastatic colorectal cancer (Hurwitz et al., 2004). This was the first report on successful tumor therapy with antiangiogenic treatment strategies, which clearly documented the importance of angiogenesis in colorectal cancer pathogenesis.

### Endothelial Cell and Inflammatory Cell Interaction

As yet, the effect of inflammation on angiogenesis in colorectal carcinoma has not been investigated in detail. Blood vessels can be detected in inflammatory areas of colorectal carcinomas. In addition, angiogenesis is a characteristic feature of inflammatory tissues. Both observations apparently suggest that inflammation may positively contribute to angiogenesis in colorectal carcinoma. However, it is well known that inflammatory cytokines such as interleukin (IL)-1beta, tumor necrosis factor (TNF)-alpha and interferon (IFN)-gamma are potent inhibitors of endothelial cell proliferation and invasion in vitro (Cozzolino et al., 1990; Frater-Schroder et al., 1987; Friesel et al., 1987; Guenzi et al., 2001; Guenzi et al., 2003; Schweigerer et al., 1987). In addition, inflammatory cytokines have been shown to inhibit angiogenesis in different animal models in vivo (Cozzolino et al., 1990; Fathallah-Shaykh et al., 2000; Norioka et al., 1994; Yilmaz et al., 1998). In contrast, in some other animal models an induction of angiogenesis has been observed in the presence of inflammatory cytokines (Frater-Schroder et al., 1987; Gerol et al., 1998; Mahadevan et al., 1989; Montrucchio et al., 1994; Torisu et al., 2000) and it has been reported that according to their concentrations inflammatory cytokines may act either as pro- or anti-angiogenic molecules in the same model system (Fajardo et al., 1992).

The antiangiogenic effect of inflammatory cytokines may be caused by their direct inhibitory effects on endothelial cell proliferation and invasion (Guenzi et al., 2001; Guenzi et al., 2003; Naschberger et al., 2005). The angiogenic effects of inflammatory cytokines have been attributed to indirect mechanisms, via the recruitment of monocytes into tissues that in turn may release angiogenic factors (Fajardo et al., 1992; Frater-Schroder et al., 1987; Joseph and Isaacs, 1998; Montrucchio et al., 1994) or to the induction of basic fibroblast growth factor (bFGF) or VEGF expression in resident cells (Samaniego et al., 1997; Torisu et al., 2000). Altogether, these results indicate that angiogenesis in colorectal carcionoma may critically depend on the specific micromilieu generated by the interplay of tumor cells, inflammatory cells and endothelial cells. This may significantly vary in different tumor stages but also in different areas of the same tumor. Thus, angiogenesis may be activated in certain tumor areas/stages and inhibited in others.

The relationship of inflammation and cancer has been a matter of debate up to now. Chronic inflammatory diseases such as ulcerative colitis and Crohn's disease predispose patients for colorectal carcinoma with an up to 10-fold increased risk (reviewed in Itzkowitz and Yio, 2004; Clevers, 2004; Farrell and Peppercorn, 2002). It has been demonstrated that chronic inflammation not only triggers the progression of cancer but also the initiation. For example, chronic inflammation is believed to be responsible for the neoplastic transformation of intestinal epithelium (reviewed in Itzkowitz and Yio, 2004). In contrast, acute inflammation of the Th1-type is considered as a host response which antagonizes tumor progression. Efforts have been undertaken to induce acute inflammation in tumor patients by e.g., systemic IL-2 immunotherapy in renal cell carcinoma where but the responses were low (Negrier et al., 1998). The relationship of inflammation, tumor initiation/progression and angiogenesis in the sporadic CRC remains largely unclear.

Recently, a concept determined as “immunoangiostasis” has been introduced by Stricter and colleagues. It was described that under certain pathological conditions in the tissue a micromilieu is established that corresponds to an IFN-γ-dependent (Th-1-like) immune reaction which finally leads to an intrinsic angiostatic reaction. This angiostatic activity has been largely attributed to the induction of the anti-angiogenic chemokines CXCL9 (monokine induced by IFN-γ [MIG]), CXCL10 (IFN-γ inducible protein-10 [IP-10]) and CXCL11 (IFN-inducible T-cell a chemoattractant [I-TAC]) by IFN-γ. These chemokines belong to the CXC chemokine subfamily that all lack a so called “ELR” amino acid motif (Glu-Leu-Arg) (Strieter et al., 2005b). Currently, the anti-angiogenic chemokines consist of five members that are CXCL4 (platelet factor-4 [PF-4]) (Spinetti et al., 2001), CXCL9, CXCL10, CXCL11 and CXCL13 (B-cell chemoattractant-1 [BCA-1]) (Romagnani et al., 2004). All angiostatic chemokines except from CXCL4 are induced by IFN-gamma (Romagnani et al., 2001). CXCL4, CXCL9, CXCL10 and CXCL11 bind to the same receptor, namely CXCR3 that is expressed by CD4 and CD8 lymphocytes, B cells, NK cells and endothelial cells. The CXCR3 receptor exists in two alternatively spliced variants CXCR3-A and CXCR3-B and the latter is responsible for the anti-angiogenic action of the chemokines (Lasagni et al., 2003).

One of the most abundant proteins induced by IFN-γ is the guanylate binding protein-1 (GBP-1) that belongs to the family of large GTPases (Prakash et al., 2000; Cheng et al., 1983; Naschberger et al., 2005).

The inventors demonstrated that GBP-1 is not only induced by IFN-γ, rather by a group of inflammatory cytokines (IFN-α/γ, interleukin [IL]-1α/β and tumor necrosis factor [TNF]-α) (Lubeseder-Martellato et al., 2002; Naschberger et al., 2004). GBP-1 expression was preferentially associated with endothelial cells (EC) in vitro and in vivo (Lubeseder-Martellato et al., 2002) and GBP-1 was shown to regulate and mediate the inhibition of proliferation induced by inflammatory cytokines (IC) in endothelial cells as well as their invasive capacity (Guenzi et al., 2001; Guenzi et al., 2003). The protein was established as a histological marker of normal endothelial cells that are activated by IC and display an anti-angiogenic phenotype.

Thus, inflammation and angiogenesis are important stroma reactions of colorectal carcinoma (CRC). Inflammation can exert pro- or antiangiogenic activity. These effects of inflammation may vary in different patients. Pre-therapeutic differentiation of angiogenic and angiostatic inflammation therefore may clearly improve the efficacy of antiangiogenic but also of other forms of therapy of CRC. In addition, this approach may also be adequate to predict therapy response in other diseases.

### SUMMARY

Therefore, it is an object of the invention to provide a means and method for the detection, prediction and/or diagnosis of an angiostatic tumor stage/tumor area of colorectal carcinoma in a patient. It is a further object of the present invention to provide molecular markers to predict responses to therapy of patients with colorectal carcinoma and also other diseases (e.g., breast carcinoma, lung canarcinoma also). It is a further object of the present invention to provide substances, which are suitable for the treatment of colorectal carcinoma.

These objects are achieved by the subject-matter of the independent claims. Preferred embodiments are set forth in the dependent claims.

The inventors investigated whether guanylate binding protein-1 (GBP-1) may be a marker of angiostatic inflammation in CRC, because it characterizes endothelial cells exposed to inflammatory cytokines and mediates the direct antiangiogenic effects of these factors.

It was found that GBP-1 is strongly expressed in endothelial cells and monocytes in the desmoplastic stroma of some CRC. Transcriptome analysis of GBP-1-positive and -negative CRC (n=24) demonstrated that GBP-1 is highly significant (p<0.001) associated with an interferon-γ (IFN-γ)-dominated micromilieu and high expression of antiangiogenic chemokines (CXCL9, CXCL10, CXCL11). Corresponding conditions have been referred to as immunoangiostasis (IAS) recently. The association of GBP-1 and angiostasis was confirmed by the detection of an inverse relation of GBP-1 expression and endothelial cell proliferation in the tumor vessels. Moreover, this association was affirmed in an independent disease, namely caseating tuberculosis. This avascular disease is the prototype of highly active IAS and exhibited an extremely robust expression of GBP-1. Most importantly, an immunohistochemical analysis of 388 colonic carcinoma tissues showed that GBP-1 was associated with a highly significant (p<0.001) increased (16.2%) cancer-related 5-year survival of the patients. Moreover, the relative risk of cancer-related death was lowered by 50% in GBP-1-positive colonic carcinoma.

It is shown herein that GBP-1 is a novel marker, among others, and active component of IAS in CRC and it is demonstrated that GBP-1-associated IAS is beneficial for the survival of CRC patients. GBP-1 expression along with the coexpression of several other markers may be a valuable prognostic marker to identify tumors with high intrinsic antiangiogenic activity and GBP-1-positive CRC will differentially respond to antiangiogenic therapy but also to all other forms of therapy as compared to GBP-1-negative CRC. The induction of GBP-1-associated IAS may be a promising approach for the clinical treatment of CRC.

At present an angiostatic stage is not considered to exist in CRC. The inventors have demonstrated that such a stage exists, concommitantly with the availability of means and methods, which allows one to detect this stage.

The availability of a method to detect patients with “angiostatic CRC” has three major advantages: (1) It allows at an early stage to apply appropriate treatment strategies to these patients. (2) The specific selection of patients will improve the clinical efficacy of antiangiogenic therapy but likely also to other forms of therapy. (3) Improved selection criteria for therapy responsive patients will significantly reduce the costs for the health system.

Specific forms of therapy which are referred to above include the following but also additional drugs which are used for treatment of colorectal carcinoma but also additional diseases:

(1) Direct and indirect inhibitors of angiogenesis, immunomodulatory molecules and other drugs (clinically approved): monoclonal antibodies (e.g., bevacizumab, cetuximab, ranibizumab, panitumumab), tyrosine kinase inhibitors (e.g., erlotinib, sunitinib/SU11248, sorafenib, temsirolimus), aptamers (e.g., pegaptanib), endogenous angiogenesis inhibitors (e.g., endostatin), thalidomide, paclitaxel, celecoxib, bortezomib, trastuzumab, lenalidomid.
(2) Direct and indirect inhibitors of angiogenesis, immunomodulatory molecules and other drugs (clinically non-approved, in clinical trial): e.g., PTK787, SU5416, ABT-510, CNGRC peptide TNF-alpha conjugate, cyclophosphamide, combretastatin A4 phosphate, dimethylxanthenone acetic acid, docetaxel, LY317615, soy isoflavone, ADH-1, AG-013736, AMG-706, AZD2171, BMS-582664, CHIR-265, pazopanib, PI-88, everolimus, suramin, XL184, ZD6474, ATN-161, cilenigtide.

Altogether, the invention will contribute to predict therapy responses to a variety of different drugs in different diseases. In addition, the invention will contribute an important tool to the development of improved treatment strategies for cancer, which are considering the specific cellular activation phenotype predominating in individual patients to gain optimal therapeutic success.

### DETAILED DESCRIPTION

According to a first aspect, the present invention provides a microarray for the detection of an angiostatic tumor stage/tumor area of colorectal carcinoma in a patient, wherein the microarray comprises gene probes capable of specifically hybridizing to the nucleic acids according to GENE Nos. 1-108 (see Table 4) or derivatives thereof, wherein the array comprises gene probes hybridizing to a subset of at least 4 of the above nucleic acid sequences, and further, wherein the array comprises gene probes specifically hybridizing to the nucleic acid sequences of GENE Nos. 1, 4, 8 and 41 (corresponding to SEQ ID NOs: 5, 1, 3, and 7, respectively).

The term “microarray” as used herein is meant to comprise DNA microarrays as well as protein microarrays.

A DNA microarray in the meaning of the present invention (also commonly known as gene or genome chip, DNA chip, or gene array) is a collection of microscopic DNA spots attached to a solid surface, such as glass, plastic or silicon chip forming an array for the purpose of expression profiling, monitoring expression levels for several genes simultaneously.

The affixed DNA segments are known and termed herein as probes, and many of them can be used in a single DNA microarray. The term gene probe generally means a specific sequence of single-stranded DNA or RNA. The term “probe” generally is here defined as a nucleic acid which can bind to a target nucleic acid via one or more kind of chemical binding, usually via complementary base pairing which usually utilizes hydrogen bonds. A probe thus is designed to bind to, and therefore single out, a particular segment of DNA to which it is complementary. Therefore, it is sufficient for the purposes of the present invention that the gene probe only hybridizes to a small part of the nucleic acid sequences indicated herein.

For performing an analysis, the following approach might be chosen:

At first, RNA is extracted from a patient sample, than the RNA is transcribed into cDNA or cRNA following purification and/or amplification steps. The cDNA or cRNA obtained may be provided with labels, if required. These nucleic acids in the next step are hybridized with the microarray as defined herein, whereby labelled cDNA or cRNA pieces are binding to its complementary counterpart on the array. Following washing away unbound cDNA or cRNA pieces, the signal of the labels in each position of the microarray may be recorded by a suitable device.

As mentioned above and as it can be derived from Table 4, GBP-1 (GENE No. 41; SEQ ID NOs: 7/8) is a powerful biomarker of an angiostatic immune reaction in colorectal cancer (CRC) and might already serve alone as a valuable tool for detecting an angiostatic tumor stage in a patient suffering from CRC. However, it also turned out that an even more valuable tool can be established, if the expression of at least three additional markers is evaluated, being the genes corresponding to GENE Nos. 1, 4, and 8 (CXCL11, CXCL9 and CXCL10; SEQ ID NOs: 5/6, 1/2, and 3/4, respectively). Interestingly, these three chemokines CXCL9, CXCL10, CXCL11 were among the 15 highest upregulated genes in GBP-1-positive tumors and were also found to be clearly higher expressed in GBP-1-positive as compared to -negative tumors. Thus, they can serve to enhance the sensitivity of detecting an angiostatic stage in an individual patient.

Therefore, it is an essential element of the invention that the microarray is at least comprising gene probes which are capable of hybridizing to the nucleic acid sequences of GENE Nos. 1, 4, 8 and 41 (corresponding to SEQ ID NOs: 5, 1, 3, and 7, respectively).

Although it is sufficient that the array contains these probes in order to achieve the object of the present invention, i.e. to detect, whether an angiostatic stage is present in an individual CRC patient or not (in order to subsequently chose the appropriate therapeutical steps), additional gene probes may be included which are capable of hybridizing to further nucleic acids selected from the group of GENE Nos. 1-108.

Among these, further subgroups of genes preferably may be selected, specifically those, which are expressed in increased levels in GBP-1-positive CRC and have been shown to play an important role in the regulation of the cellular response to IFN: GENE Nos. 1, 4, 8, 14, 25, 26, 41, 54 59, 65, 76, 81, 105, 106, 107, 108 (see Table 4) and those whose expression is more than 10fold increased in GBP-1 positive CRC: 1-17. Further subgroups may be identified as GENE Nos. 26, 54, 59, 65, 81, 105, 106, 107, and/or 108. It is noted that it is also preferred to additionally use these nucleic acids alone or in combination which each other, for example, and more preferred, subgroups GENE Nos. 26, 54, 59, 65, 81 and/or 105, 106, 107, 108.

In a further embodiment, the microarray may additionally contain gene probes capable of specifically hybridizing to at least one of the nucleic acids according to GENE Nos. 109-157 (see Table 5), being 49 gene probes of genes with increased expression in hGBP-1-negative CRC. These additional nucleic acid sequences and the respective gene probes hybridizing to them may be used as “negative” control in order to further enhance the predictive value of the microarray.

Because it has been shown that vascular endothelial cell growth factor (VEGF) and basic fibroblast growth factor (bFGF) are major regulators of angiogenesis, the microarray may preferably also contain probes also to these genes. Both genes were not found to be differentially expressed in GBP-1-positive and -negative CRC, because they are generally expressed in increased levels in all CRC as compared to healthy tissues. However, due to their specific activity which antagonizes the effects of GBP-1-associated immunoangiostasis, probes for VEGF (including VEGF-A, VEGF-B, VEGF-C, VEGF-D) and bFGF and all splice variants of the respective genes will be used as a standard to determine basic angiogenic activation. To these goal the probes for VEGF and bFGF will be applied in combination with all gene groups mentioned above: namely GENE Nos. 1-108 or 109-157; GENE Nos. 1, 4, 8, 14, 25, 26, 41, 59, 65, 76, 81, 105, 106, 107, 108; or GENE Nos. 1-17.

The microarray of the present invention additionally may contain appropriate control gene probes, e.g., actin or GAPDH. Those can be included as control gene probes to determine relative signal intensities.

In a preferred embodiment, the gene probes used in the microarray of the invention are oligonucleotides, cDNA, RNA or PNA molecules.

As mentioned above, the nucleic acids as defined above preferably are labelled in order to allow a better detection of their binding to the corresponding gene probe on the array. Preferably, such a label is selected from the group consisting of a radioactive, fluorescence, biotin, digoxigenin, peroxidase labelling or a labelling detectable by alkaline phosphatase.

In a further embodiment, the gene probes of the array may be bound to a solid phase matrix, e.g., a nylon membrane, glass or plastics.

In a second aspect, the present invention is directed to a protein microarray, capable of detecting at least a subset of four amino acid sequences of a group of amino acid sequences corresponding to the nucleic acid sequences of GENE Nos. 1-108, wherein the array is capable of at least detecting the amino acids corresponding to the nucleic acid sequences of GENE Nos. 1, 4, 8 and 41 (corresponding to SEQ ID NOs: 5, 3, 1, and 7, respectively).

Or in other words, the protein microarray is capable of detecting all amino acids corresponding to nucleic acid sequences and subgroups as defined hereinabove.

In the protein microarray of the present invention, the array preferably is an antibody microarray or a Western-blot microarray.

An antibody microarray is a specific form of a protein microarray, i.e. a collection of capture antibodies are spotted and fixed on a solid surface, such as glass, plastic and a silicon chip for the purpose of detecting antigens.

The term “antibody”, is used herein for intact antibodies as well as antibody fragments, which have a certain ability to selectively bind to an epitope. Such fragments include, without limitations, Fab, F(ab′)₂, ScFv and Fv antibody fragment. The term “epitop” means any antigen determinant of an antigen, to which the paratop of an antibody can bind. Epitop determinants usually consist of chemically active surface groups of molecules (e.g., amino acid or sugar residues) and usually display a three-dimensional structure as well as specific physical properties.

The antibodies according to the invention can be produced according to any known procedure. For example the pure complete protein according to the invention or a part of it can be produced and used as immunogen, to immunize an animal and to produce specific antibodies.

The production of polyclonal antibodies is commonly known. Detailed protocols can be found for example in Green et al, Production of Polyclonal Antisera, in Immunochemical Protocols (Manson, editor), pages 1-5 (Humana Press 1992) und Coligan et al, Production of Polyclonal Antisera in Rabbits, Rats, Mice and Hamsters, in Current Protocols In Immunology, section 2.4.1 (1992). In addition, the expert is familiar with several techniques regarding the purification and concentration of polyclonal antibodies, as well as of monoclonal antibodies (Coligan et al., Unit 9, Current Protocols in Immunology, Wiley Interscience, 1994).

The production of monoclonal antibodies is as well commonly known. Examples include the hybridoma method (Kohler and Milstein, 1975, Nature, 256:495-497, Coligan et al., section 2.5.1-2.6.7; and Harlow et al., Antibodies: A Laboratory Manual, page 726 (Cold Spring Harbor Pub. 1988)), the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96).

In brief, monoclonal antibodies can be attained by injecting a mixture which contains a protein/peptide into mice/rats. The antibody production in the mice/rats is checked via a serum probe. In the case of a sufficient antibody titer, the mouse/rat is sacrificed and the spleen is removed to isolate B-cells. The B cells are fused with myeloma cells resulting in hybridomas. The hybridomas are cloned and the clones are analyzed. Positive clones which contain a monoclonal antibody against the protein are selected and the antibodies are isolated from the hybridoma cultures. There are many well established techniques to isolate and purify monoclonal antibodies. Such techniques include affinity chromatography with protein A sepharose, size-exclusion chromatography and ion exchange chromatography. Also see for example. Coligan et al., section 2.7.1-2.7.12 and section “Immunoglobulin G (IgG)”, in Methods In Molecular Biology, volume 10, pages 79-104 (Humana Press 1992).

In a third aspect, the present invention provides an inhibitor or modulator of one or more of the nucleic acids of GENE Nos. 1-108, or of the amino acids expressed therefrom. Such substances may be used for the treatment of colorectal carcinoma.

The inhibitor or modulator is preferably selected from the group consisting of an antisense nucleic acid, a ribozyme, double stranded RNA, siRNA, microRNA an antibody, a receptor, a mutated transdominant negative variant of the protein, a peptide and a peptidomimetic.

In a fourth aspect, the invention provides a pharmaceutical composition, which comprises an inhibitor/modulator as defined above and a pharmaceutically acceptable carrier.

The active compounds of the present invention are preferably used in such a pharmaceutical composition, in doses mixed with an acceptable carrier or carrier material, that the disease can be treated or at least alleviated. Such a composition can (in addition to the active component and the carrier) include filling material, salts, buffer, stabilizers, solubilizers and other materials, which are known state of the art.

The term “pharmaceutically acceptable” is defined as non-toxic material, which does not interfere with effectiveness of the biological activity of the active compound. The choice of the carrier is dependent on the application.

The pharmaceutical composition can contain additional components which enhance the activity of the active component or which supplement the treatment. Such additional components and/or factors can be part of the pharmaceutical composition to achieve a synergistic effect or to minimize adverse or unwanted effects.

Techniques for the formulation or preparation and application/medication of compounds of the present invention are published in “Remington's Pharmaceutical Sciences”, Mack Publishing Co., Easton, Pa., latest edition. A therapeutically effective dose relates to the amount of a compound which is sufficient to improve the symptoms, for example a treatment, healing, prevention or improvement of such conditions. An appropriate application can include for example oral, dermal, rectal, transmucosal or intestinal application and parenteral application, including intramuscular, subcutaneous, intramedular injections as well as intrathecal, direct intraventricular, intravenous, intraperitoneal or intranasal injections. The intravenous injection is the preferred treatment of a patient.

A typical composition for an intravenous infusion can be produced such that it contains 250 ml sterile Ringer solution and for example 10 mg protein compound. See also Remington's Pharmaceutical Science (15. edition, Mack Publishing Company, Easton, Ps., 1980).

The active component or mixture of it in the present case can be used for prophylactic and/or therapeutic treatments.

A fifth aspect of the present invention is directed to an ex vivo method for the diagnosis of an angiostatic tumor stage/tumor area in a CRC patient comprising the steps of:
(a) providing a sample of the patient;
(b) extracting RNA from the sample;
(c) optionally transcribing RNA to cDNA or cRNA;
(d) detecting, whether at least four nucleic acid sequences selected from the group consisting of GENE Nos. 1-108 are present in the sample, and whether the sample contains at least the nucleic acid sequences of GENE Nos. 1, 4, 8 and 41 (corresponding to SEQ ID NOs: 5, 1, 3, and 7, respectively);
(e) wherein the presence of said nucleic acids is indicative for the presence of an angiostatic tumor stage/tumor area of CRC in said patient.

The sample used in this method preferably is a CRC tissue sample or a cell lysate or a body fluid sample.

The detection preferably is performed by PCR, more preferably by RT-PCR, most preferably multiplex RT-PCR. The PCR method has the advantage that very small amounts of DNA are detectable. Dependent on the to be analyzed material and the equipment used the temperature conditions and number of cycles of the PCR have to be adjusted. The optimal conditions can be experimentally determined according to standard procedures.

Multiplex-PCR conditions for the simultaneous detection of GBP-1, CXCL9, CXCL10 and CXCL11 might be set as follows:

Reaction mixture:

cDNA 1 μl (corresponding to 50 ng total-RNA)

dNTP 200 μM

GBP-1, CXCL10 and CXCL11 primer each 0.4 μM, CXCL9 primer 0.8 μM

10× FastStart High Fidelity Reaction Buffer (Fa. Roche) 5 μl

FastStart High Fidelity Enzyme (Fa. Roche) 0.5 μl

Ad 50 μl Millipore-H₂O

### Program:

95° C. 2 min 1×

95° C. 30 sec 35×

55° C. 30 sec

72° C. 30 sec

72° C. 4 min 1×

4° C. unlimited

⅓ of the PCR-product are applied to a agarose gel.

The during the PCR amplification accrued, characteristic, specific DNA fragments can be detected for example by gel electrophoretic or fluorimetric methods with the DNA labeled accordingly. Alternatively, other appropriate, known to the expert, detection systems can be applied.

The DNA or RNA, especially mRNA, of the to be analyzed probe can be an extract or a complex mixture, in which the DNA or RNA to be analyzed are only a very small fraction of the total biological probe. This probe can be analyzed by PCR, e.g., RT-PCR. The biological probe can be serum, blood or cells, either isolated or for example as mixture in a tissue.

The detection is—as already outlined above—preferably performed by means of complementary gene probes. Those gene probes preferably are cDNA or oligonucleotide probes. Furthermore, these gene probes preferably are capable of hybridizing to at least a portion of the nucleic acid sequences of GENE Nos. 1-108, or to RNA sequences or derivatives derived therefrom.

According to the invention, the hybridization to the nucleic acids according to the invention is done at moderate stringent conditions.

Stringent hybridization and wash conditions are in general the reaction conditions for the formation of duplexes between oligonucleotides and the desired target molecules (perfect hybrids) or that only the desired target can be detected. Stringent washing conditions mean 0.2×SSC (0.03 M NaCl, 0.003 M sodium citrate, pH 7)/0.1% SDS at 65° C. For shorter fragments, e.g., oligonucleotides up to 30 nucleotides, the hybridization temperature is below 65° C., for example at 50° C., preferably above 55° C., but below 65° C. Stringent hybridization temperatures are dependent on the size or length, respectively of the nucleic acid and their nucleic acid composition and will be experimentally determined by the skilled artisan. Moderate stringent hybridization temperatures are for example 42° C. und washing conditions with 0.2×SSC/0.1% SDS at 42° C.

The expert can according to the state of the art adapt the chosen procedure, to reach actually moderate stringent conditions and to enable a specific detection method. Appropriate stringent conditions can be determined for example on the basis of reference hybridization. An appropriate nucleic acid or oligonucleotide concentration needs to be used. The hybridization has to occur at an appropriate temperature (the higher the temperature the lower the binding).

In a preferred embodiment, the microarray as defined above is used for the detection.

A sixth aspect of the present invention provides an ex vivo method for the diagnosis of an angiostatic tumor stage/tumor area in a CRC patient comprising the steps of:
(a) providing a sample from the patient;
(b) detecting, whether at least four amino acid sequences corresponding to the nucleic acid sequences selected from the group of GENE Nos. 1-108 are present in the sample, and whether the sample contains at least the amino acids corresponding to the nucleic acid sequences of GENE Nos. 1, 4, 8 and 41 (corresponding to SEQ ID NOs: 5, 1, 3, and 7, respectively);
(c) wherein the presence of said proteins is indicative for the presence of an angiostatic tumor stage/tumor area of CRC in said patient.

In a preferred embodiment, the detection is performed by contacting the sample with antibodies, which specifically recognize an amino acid expressed from a nucleic acid sequence of one of GENE Nos. 1-108.

Preferably, the sample is a CRC tissue sample, a cell lysate or a body fluid. The amino acid sequences are preferably detected by means of multiplex Western blot or ELISA.

The present invention will be further described with reference to the following figures and examples; however, it is to be understood that the present invention is not limited to such figures and examples.

### BRIEF DESCRIPTION OF THE DRAWINGS

FIG. 1. Coexpression of GBP-1 and interferon-induced angiostatic chemokines in colorectal carcinoma. Immunohistochemical staining of GBP-1 in (A, C) CRC tissue and (B, D) healthy mucosa tissue of two representative patients. GBP-1-positive cells are indicated by an arrow, tumor cells are labeled by an asterisk. In situ hybridization of CRC tissue sections with ³⁵S-radiolabeled GBP-1 (E, F) antisense and (G, H) sense RNA strand hybridization probes. Prominent signals were obtained with the antisense hybridization probe (complementary to GBP-1 mRNA) in the stroma of CRC, both in the (E) bright field (BF, black grains) and (F) dark field (DF, white grains) exposure. (G, H) Control hybridization with the GBP-1 sense strand RNA probe did not show specific signals. Immunohistochemical staining of (I) GBP-1, (J) CD31 and (K) CD68 in consecutive sections of CRC. Corresponding tissue areas are indicated by arrows. (L) Example of a CRC tissue negative for GBP-1 in immunohistochemistry. Magnifications: (A-D) ×850, (E-L) ×530. (M) Normalized microarray signal intensities (relative light units: RLU) of GBP-1, CXCL9 and CXCL11 expression in GBP-1-positive (GBP-1↑, n=12) and GBP-1-negative CRC (GBP-1↓, n=12). The tumors are given at corresponding positions in each diagram. (N) Semi-quantitative RT-PCR of GBP-1 coregulated genes (CXCL10, CXCL9, CXCL11, IDO, MCP-2, Mx1, OAS2 and granzyme A) in three different GBP-1-positive (GBP-1↑) and GBP-1-negative (GBP-1↓) CRC. Decreasing amounts of cDNA (undiluted, 1/10, 1/100 and 1/1000) of the different tumors were subjected to each PCR. Amplification of GAPDH demonstrates that equal amounts of cDNA were used from each tumor.

FIG. 2. GBP-1 is associated with angiostasis and increased cancer-related 5-year survival in colorectal carcinoma. (A) CXCR3-B expression was analyzed with semi-quantitative RT-PCR in three GBP-1-positive (GBP-1↑) and GBP-1-negative (GBP-1↓) CRC. CDNA was subjected in decreasing amounts (undiluted, 1/10, 1/100 and 1/1000) to the PCR. Amplification of GAPDH demonstrates that equal amounts of cDNA of the different tumors were used. Immunohistochemical staining of (B, C) GBP-1, (D, E) CD31 and (F, G) Ki-67 (proliferation-associated antigen) on consecutive sections of GBP-1-positive (+) or negative (−) vessels. Corresponding cells are indicated by arrows. Immunohistochemical detection of (H, I) GBP-1, (J) CD68 and (K) CD31 in caseating tuberculosis. (H) Overview (GBP-1 positive cells, arrows) and (I, J, K) consecutive sections (corresponding cell indicated by arrows) of the field indicated in (H). Magnifications (B-G) ×850, (H) ×85, (I-K) ×530. (L) Cancer-related 5-year survival of patients with GBP-1-positive (red, n=124) and -negative colonic carcinoma (black, n=264). The cancer-related survival is depicted by a Kaplan-Meier-Curve and 95% confidence intervals.

FIG. 3. Quantification of GBP-1 staining in the CRC tissue array. CRC tissue arrays were immunohistochemically stained for GBP-1 (brown). (A) Numbers of positive cells (0, negative; 1, <50%; 2, ˜50%; 3, >50%) and (B) GBP-1 expression levels (−, negative; +, weak; ++, middle; +++, high) were determined. Magnification ×215.

FIG. 4. The anti-angiogenic chemokines CXCL9-11 are GBP-1-coregulated genes in the colorectal carcinoma (CRC). A multiplex-RT-PCR for CXCL9-11 and GBP-1 using RNA from seven different colorectal carcinoma patients was performed. Patients were categorized as “GBP-1-negative” or “GBP-1-positive” according to immunohistochemistry results. As a negative (Neg. ctrl.) and positive control (Pos. ctrl.) RNA from unstimulated and IFN-γ-stimulated HUVEC, respectively was used in parallel.

### EXAMPLES

### Example 1

### GBP-1 Indicates an Intrinsic Angiostatic Immune Reaction in Colorectal Carcinoma

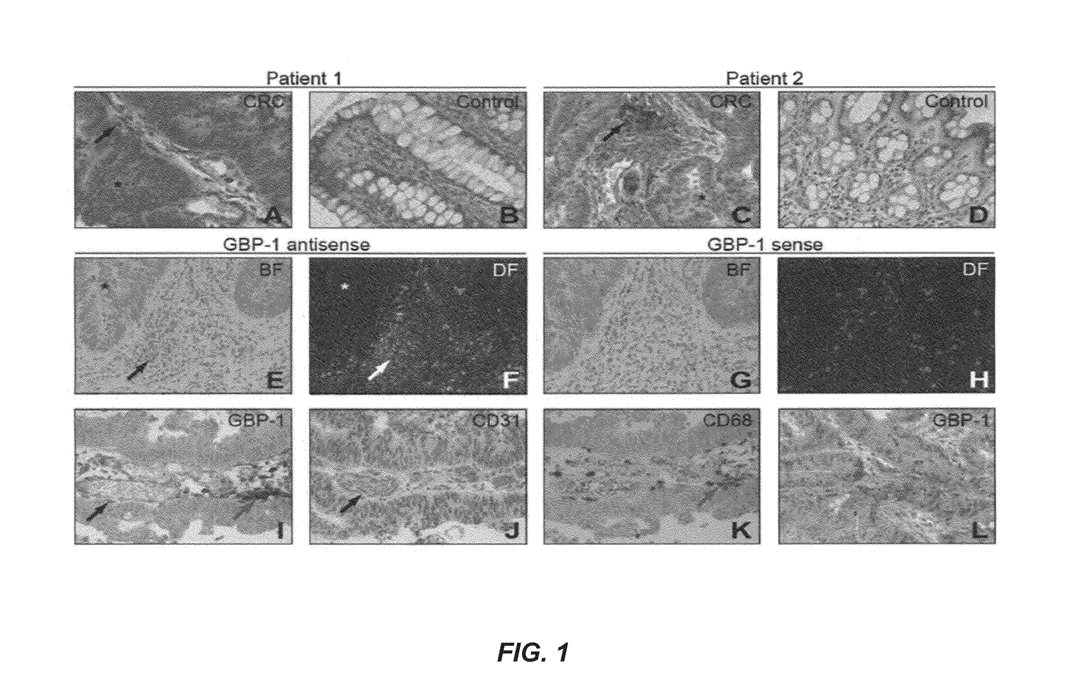

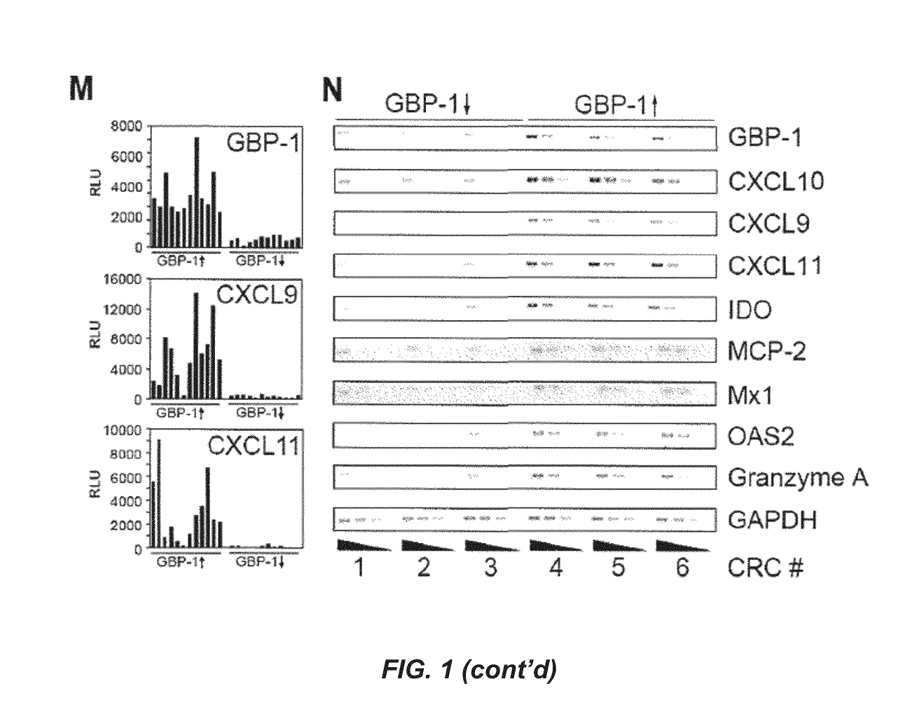

Robust expression of GBP-1 was detected in the desmoplastic stroma of colorectal carcinomas obtained from two different patients by immunohistochemistry (FIG. 1A, C, arrows). GBP-1 was not expressed in the tumor cells (FIG. 1A, C, asterisk) and in adjacent tumor free mucosa of the colon (FIG. 1B, D). These results were confirmed by in situ hybridization. With a GBP-1 mRNA specific probe strong signals were obtained in the tumor stroma exclusively (FIG. 1E, F, arrows, bright field [BF] and dark field [DF] of the same tissue section) but not in the tumor cell area (FIG. 1E, F, asterisk). No unspecific signals were obtained when the respective negative control probe was used (FIG. 1G, H; BF and DF of the same tissue section). Immunohistochemical staining of GBP-1, CD31 and CD68 in consecutive tumor sections demonstrated that GBP-1 (FIG. 1I) is expressed in endothelial cells (FIG. 1I, J, black arrows) and immune cells, most likely monocytes/macrophages (FIG. 1I, K, red arrows). In contrast, CRC obtained from three other patients did not express GBP-1 (FIG. 1L).

### Example 2

### GBP-1 Indicates an Intrinsic Angiostatic Immune Reaction in Colorectal Carcinoma

To characterize the GBP-1-associated micromilieu, 12 GBP-1-positive und 12 GBP-1-negative CRC of patients with closely matched clinical parameters (Table 1, lower panel) were identified by immunohistochemistry and subjected to a transcriptome analysis (HG-U133A, Affymetrix, 22,215 probe sets). Signals were normalized and listed according to their probability to reflect differential expression (p<0.05), significant signal intensity (>300 RLUs) and robust upregulation of expression (>4-fold) in GBP-1-positive tumors. 104 genes fulfilled these criteria (Table 4). Most of these genes were either well-known IFN-induced genes, and/or encoded chemokines or immune reaction-associated genes (Table 4). Interestingly, the three major angiostatic chemokines (CXCL9, CXCL10, CXCL11: table 4, shaded) (Strieter et al., 2005b; Romagnani et al., 2004) were among the eight most strongly upregulated genes in GBP-1-positive tumors. Expression of angiogenic growth factors such as VEGF and basic fibroblast growth factor (bFGF) was not increased in GBP-1-positive CRC.

High reproducibility of the microarray analyses is demonstrated by the fact that within the groups of GBP-1-positive and -negative tumors highly reproducible results were obtained for each gene as shown exemplarily for GBP-1, CXCL9 and CXCL11 (FIG. 1M). In addition, semi-quantitative RT-PCR confirmed the microarray results showing that each of the three angiostatic chemokines (CXCL10, CXCL9, CXCL11) and of five additional IFN-γ-induced and/or immune reaction-associated genes [IFN-γ-inducible indoleamine 2,3-dioxygenase (IDO), monocyte chemotactic protein-2 (MCP-2), Mx1, 2′-5′-oligoadenylate synthetase-2 (OAS2) and granzyme A] were higher expressed in GBP-1-positive as compared to GBP-1-negative tumors (FIG. 1N).

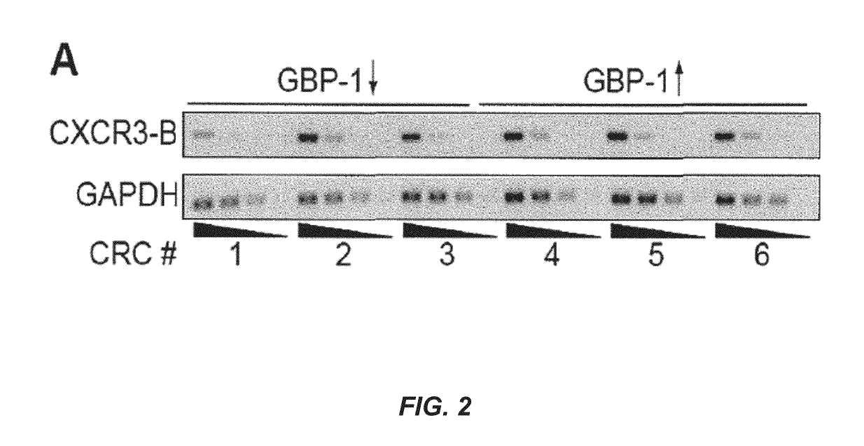

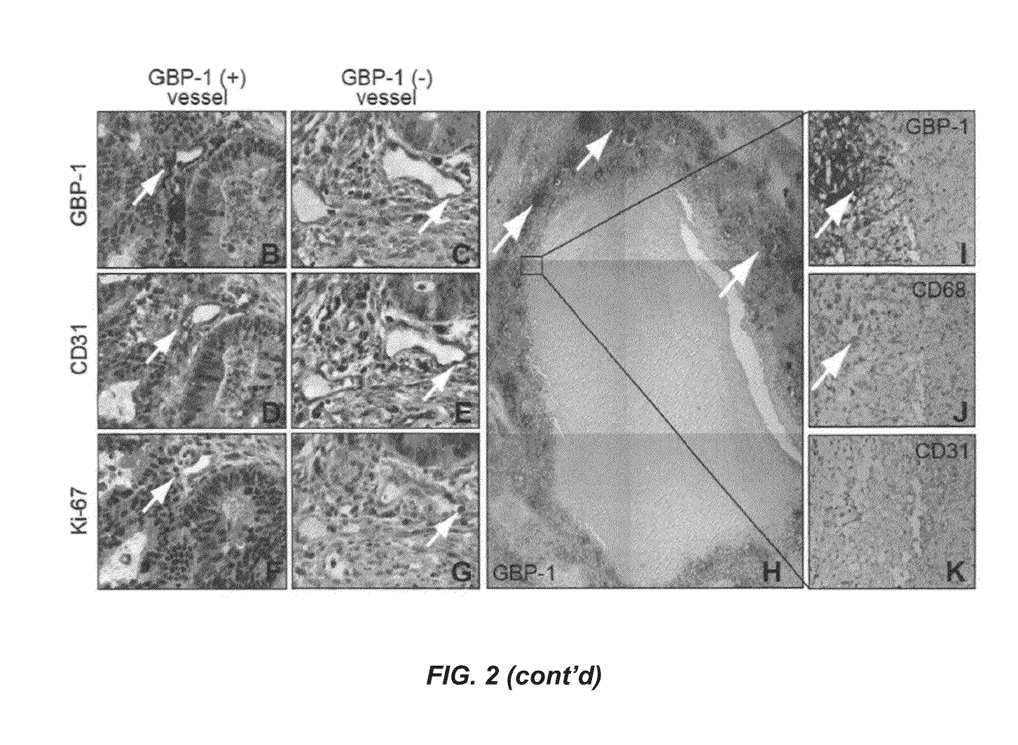

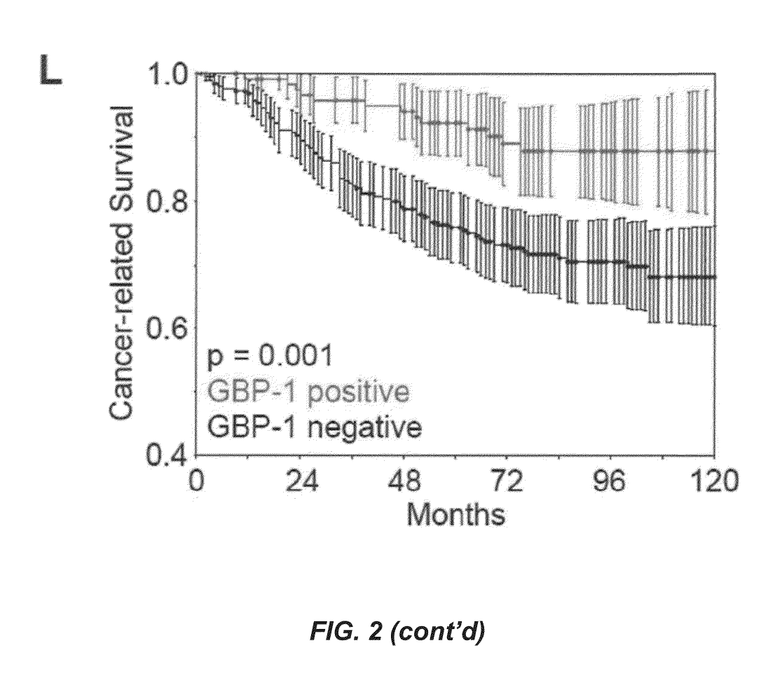

An IFN-γ-dominated micromilieu characterized by the presence of the angiostatic chemokines has recently been described to regulate an intrinsic angiostatic immune reaction (IAR) (Strieter et al., 2005a; Strieter et al., 2006; Stricter et al., 2004; Stricter et al., 2005b). The antiangiogenic chemokines CXCL9-11 inhibit angiogenesis via the chemokine receptor CXCR3-B (Lasagni et al., 2003; Ehlert et al., 2004). RT-PCR showed that this receptor is constitutively expressed in both, GBP-1-positive and -negative CRC (FIG. 2A, CXCR3-B). Therefore, angiostasis can be induced in case CXCL9-11 are present. In addition, a negative correlation of GBP-1 expression and vessel proliferation supported the presence of angiostasis in GBP-1-positive tumors (FIG. 2B, D, F, arrows). Proliferating Ki-67-positive endothelial cells were exclusively detected in GBP-1-negative vessels but never in GBP-1-positive vessels (FIG. 2C, E, G, arrows; red nuclear Ki-67 staining indicates a proliferating endothelial cell). Finally, we challenged the concept that GBP-1 is associated with an intrinsic angiostatic immune reaction in a different disease. Caseating tuberculosis is the prototypic disease of IAR (Strieter et al., 2005a; Strieter et al., 2005b). This is most evident by the almost complete absence of blood vessels in the involved lung tissue. Immunohistochemical stainings of lung biopsies with caseating tuberculosis showed a robust GBP-1 signal (FIG. 2H, I, arrows). In agreement with the angiostatic conditions, endothelial cells were only rarely detected (FIG. 2K) and GBP-1-positive cells were predominantly macrophages (FIG. 2J, arrow).

In addition, 49 genes were identified, which were significantly increased in GBP-1-negative tumors (Table 5).

### Example 3

### GBP-1-Associated Immunoangiostasis Elongates Survival of Colorectal Carcinoma Patients

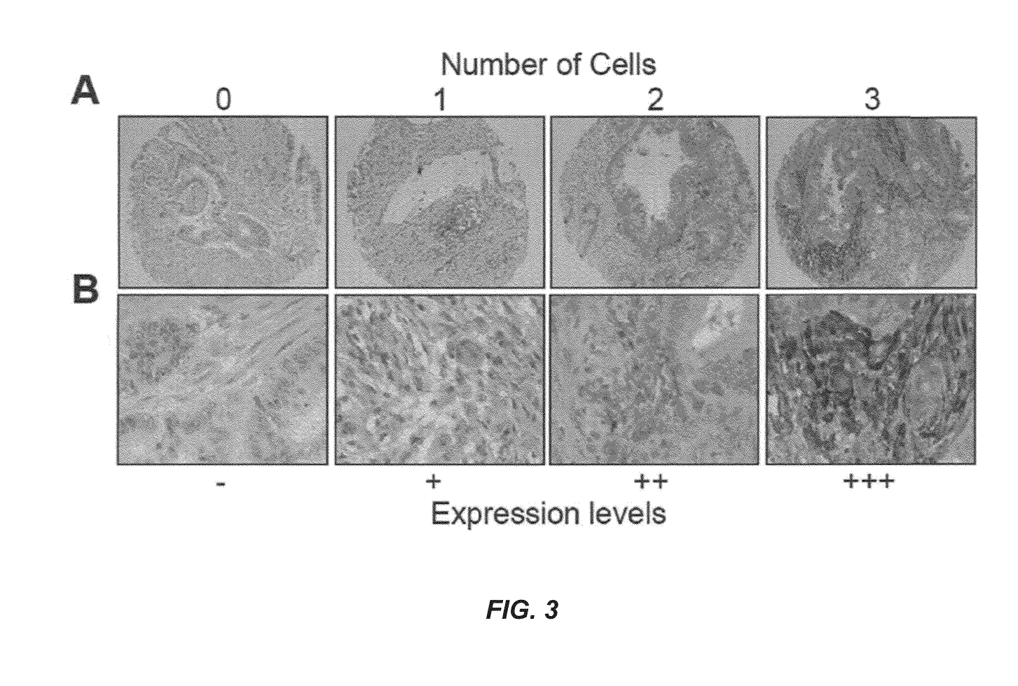

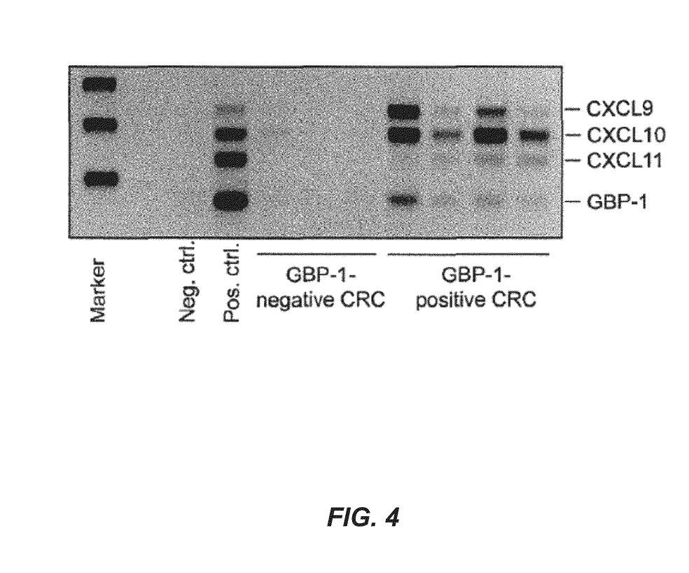

GBP-1 expression in UICC stage II-IV colonic carcinoma (n=388) was investigated by immunohistochemical tissue array technology (Tables 1 and 2). Nine different areas of each tumor were analyzed. Numbers of GBP-1-positive cells and expression levels were quantitatively determined (FIG. 3). GBP-1 was expressed in 32% of all tumors (Table 1, GBP-1 expression in the stroma) and was highly significant (p<0.001) associated with the early tumor stage (Table 2, see Stage and Regional Lymph Nodes). A considerably larger fraction of GBP-1-positive colonic carcinomas were UICC stage II (64.6%) and did not show lymph node metastasis (67.7% pN0) as compared to GBP-1-negative tumors (42.8% UICC II, 45.1% pN0). In contrast, GBP-1-negative tumors were more often in progressed UICC IV stage (11%) and showed metastasis in more than three lymph nodes (22.7% pN2) as compared to GBP-1-positive tumors (5.6% UICC IV, 12.1% pN2). Other clinical parameters such as primary tumor (pT-classification), histopathological grading or extramural venous invasion did not correlate significantly with GBP-1 expression (Table 2). The association with the UICC II stage was significant for all GBP-1-positive tumors, irrespectively of the absolute number of GBP-1-expressing cells and of GBP-1-expression level (Table 6, p value).

Interestingly, patients with GBP-1-positive colonic carcinoma had a highly significant (p<0.001) increased cancer-related 5-year survival rate of 16.2% in univariate analysis (Table 3, upper panel; FIG. 2L). Other well-established prognostic factors such as UICC stage, pT- and pN-status or extramural venous invasion did also correlate with increased survival confirming the representative value of this study group (Table 3). Most importantly, multivariate analysis showed that GBP-1 expression is an independent prognostic marker indicating a relative risk of cancer-related death of 0.5 as compared to colonic carcinoma patients that do not express GBP-1 (Table 3, lower panel).

### Material and Methods

### Clinical Samples

Affymetrix Array:

After informed consent was obtained, 24 patients who underwent surgery for the first manifestation of CRC were included in the study. The investigation was carried out in accordance with the Helsinki declaration. Patients who underwent preoperative radiation or chemotherapy did not participate in the study (Table 1). Patients with familial CRC (familial adenomatous polyposis, hereditary nonpolyposis CRC) were excluded. Stage (UICC 2002), sex ratio, patient age, T-, N-, M-stage, histopathological grading and tumor site were used as conventional clinicopathological parameters (Table 1, lower panel).

Tissue Array:

This study was based on the prospectively collected data of the Erlangen Registry of Colo-Rectal Carcinomas (ERCRC) from 1991 to 2001. 388 patients with the following inclusion criteria were selected: Solitary invasive colon carcinoma (invasion at least of the submucosa), localisation >16 cm from the anal verge, no appendix carcinoma; no other previous or synchronous malignant tumor, except squamous and basal cell carcinoma of the skin and carcinoma in situ of the cervix uteri; carcinoma not arisen in familial adenomatous polyposis, ulcerative colitis or Crohn's disease; treatment by colon resection with formal regional lymph node dissection at the Surgical Department of the University of Erlangen; residual tumor classification RO (no residual tumor, clinical and pathohistological examination); UICC stage II-IV 2002 (UICC (2002) TNM classification of malignant tumors. 6^{th} ed (Sobin L H, Wittekind Ch, eds). John Wiley & Sons, New York) (Table 1, upper panel). Patients who died postoperatively and patients with unknown tumor status (with respect to local and distant recurrence) at the end of the study (Jan. 1, 2006) were excluded. A total of nine punches from each of the 388 patients originating from tumor center (three punches), invasive front (three punches) and desmoplastic stroma in/adjacent to the tumor (three punches) were applied to the tissue array analysis. Median follow-up was 83 months (range 1-177). At the end of the study 88 patients (22.7%) had died of their colon carcinoma. Patient and tumor characteristics of the ERCRC patients are shown in Table 1, upper panel. Curatively resected distant metastases were located in the liver (n=29), distant lymph nodes (n=3), peritoneum (n=3), and others (n=3). The carcinomas were graded in accordance with the recommendations of the WHO using the categories low and high grade (Jass and Sobin 1989). With regard to venous invasion we distinguished between no or only intramural venous invasion (EVI negative [−]) and extramural venous invasion (EVI positive [+]). Emergency presentation was defined as the need for urgent surgery within 48 hours of admission (Soreide et al. 1997).

Caseating Tuberculosis:

Tissue sections of lung biopsies from six patients with the confirmed diagnosis caseating tuberculosis were obtained by the local pathology and areas including caseating granulomas were stained immunohistochemically.

### Immunohistochemical Staining

Staining for GBP-1, CD31, CD68 and Ki-67 was performed as previously described (Lubeseder-Martellato et al., 2002; Guenzi et al., 2001; Guenzi et al., 2003). The latter three antibodies were purchased from DAKO (Hamburg, Germany) and diluted as follows: CD31 (1:50), CD68 (1:200) and Ki-67 (1:300). Stained sections were evaluated by two independent persons. Differing results were evaluated by a third person and discussed until consensus was obtained.

### In Situ Hybridization

Biopsy specimens were processed as previously described (Stürzl et al., 1999; Stürzl et al., 1992). As a template for transcription of ³⁵S-labeled RNA sense/antisense hybridization probes full length GBP-1-encoding cDNA (M55542) was inserted into the pcDNA3.1 expression vector in sense/antisense orientation. T7 polymerase was used for in vitro transcription. After autoradiography sections were stained with haematoxylin and eosin and analyzed in the bright field (expression signals are black silver grains) and dark field (light scattering by silver grains produces white signals) with a Leica aristoplan microscope.

### RT-PCR Analysis

RT-PCR analysis was carried out by using the PCR primers (forward/reverse, 5′-3′ orientation) for both, RT-PCR and multiplex RT-PCR: GBP-1 (GENBANK®) Accession No. M55542): ATGGCATCAGAGATCCACAT (SEQ ID NO: 39), GCTTATGGTACATGCCTTTC (SEQ ID NO: 40); CXCL10 (GENBANK® Accession No. NM_{—}001565.1): AAGGATGGACCACACAGAGG (SEQ ID NO: 41), TGGAAGATGGGAAAGGTGAG (SEQ ID NO: 42); CXCL9 (GENBANK® Accession No. NM_{—}002416.1): TCATCTTGCTGGTTCTGATTG (SEQ ID NO: 43), ACGAGAACGTTGAGATTTTCG (SEQ ID NO: 44); CXCL11 (GENBANK® Accession No. AF030514.1): GCTATAGCCTTGGCTGTGATAT (SEQ ID NO: 45), GCCTTGCTTGCTTCGATTTGGG (SEQ ID NO: 46); IDO (GENBANK® Accession No. M34455): GCAAATGCAAGAACGGGACACT (SEQ ID NO: 47), TCAGGGAGACCAGAGCTTTCACAC (SEQ ID NO: 48); MCP-2 (GENBANK® Accession No. NM_{—}005623): ATTTATTTTCCCCAACCTCC (SEQ ID NO: 49), ACAATGACATTTTGCCGTGA (SEQ ID NO: 50); Mx1 (GENBANK® Accession No. NM_{—}002462.2): TACAGCTGGCTCCTGAAGGA (SEQ ID NO: 51), CGGCTAACGGATAAGCAGAG (SEQ ID NO: 52); OAS2 (GENBANK® Accession No. NM_{—}002535): TTAAATGATAATCCCAGCCC (SEQ ID NO: 53), AAGATTACTGGCCTCGCTGA (SEQ ID NO: 54); Granzyme A (GENBANK® Accession No. NM_{—}006144.2): ACCCTACATGGTCCTACTTAG (SEQ ID NO: 55), AAGTGACCCCTCGGAAAACA (SEQ ID NO: 56); CXCR3-B (GENBANK® Accession No. AF469635): AGTTCCTGCCAGGCCTTTAC (SEQ ID NO: 57), CAGCAGAAAGAGGAGGCTGT (SEQ ID NO: 58); GAPDH: AGCCACATCGCTCAGAACAC (SEQ ID NO: 59), GAGGCATTGCTGATGATCTTG (SEQ ID NO: 60).

### Affymetrix GENECHIP® Analysis

Affymetrix GENECHIP® analysis was carried out as described previously (Croner et al., 2005a; Croner et al., 2005b; Croner et al., 2004). The whole microarray experiment design, setup and results are available through ArrayExpress (http://www<<.>>ebi<<.>>ac<<.>>uk/arrayexpress/) using the access number E-MEXP-833.

### Statistical Analysis

Tissue Array:

The Kaplan-Meier method was used to calculate 5-year rates of cancer-related survival. An event was defined as “cancer-related death”, i. e. death with recurrent locoregional or distant cancer. The 95% confidence intervals (95% CI) were calculated accordingly (Greenwood et al., 1926). Logrank test was used for comparisons of survival. A Cox regression analysis was performed to identify independent prognostic factors. All factors which were found significant in univariate survival analysis were introduced in the multivariate model. 2 patients were excluded because of missing data on extramural venous invasion (n=386). Chi-square test was used to compare frequencies. A p-value of less than 0.05 was considered to be statistically significant. Analyses were performed using SPSS software version 13 (SPSS Inc., Chicago, USA).

Affymetrix Array:

Raw data derived from GENENHIP® assays were normalized by “global scaling” using Affymetrix Microarray Suite, Data Mining Tool. Signals of the 12 GBP-1-positive and 12 GBP-1-negative CRCs, respectively, were averaged and upregulated genes selected according to p<0.05, overall signal intensity >300 RLU and fold change >4.

### Tables

TABLE 1
Clinical Parameters of Colonic Carcinoma Patients Included
in Tissue Array Analysis (n = 388) and of Colorectal
Carcinoma Patients Included in Gene Chip Analysis (n = 24).
TISSUE ARRAY ANALYSIS

| | n | % |
|---|---|---|
| Sex ratio (male/female) | 232/156 = 1.5 | |
| Age median/range (years) | 64/28-91 | |
| GBP-1 Expression in | | |
| the Stroma | | |
| GBP-1-negative (−) | 264 | 68.0 |
| GBP-1-positive (+) | 124 | 32.0 |
| Tumor Site | | |
| Sigmoid colon | 186 | 47.9 |
| Descending colon | 16 | 4.1 |
| Splenic flexure | 23 | 5.9 |
| Transverse colon | 39 | 10.1 |
| Hepatic flexure | 26 | 6.7 |
| Ascending colon | 58 | 14.9 |
| Cecum | 40 | 10.3 |
| Stage (UICC 2002) | | |
| II | 193 | 49.7 |
| III | 159 | 41.0 |
| IV | 36 | 9.3 |
| Primary Tumor | | |
| pT2 | 27 | 7.0 |
| pT3 | 311 | 80.2 |
| pT4 | 50 | 12.9 |
| Regional Lymph Nodes | | |
| pN0 | 203 | 52.3 |
| pN1 | 110 | 28.4 |
| pN2 | 75 | 19.3 |
| Histopathological | | |
| Grading | | |
| Low grade (G1/G2) | 316 | 81.4 |
| High grade (G3/G4) | 72 | 18.6 |
| Extramural Venous | | |
| Invasion (EVI) | | |
| EVI (−) | 340 | 87.6 |
| EVI (+) | 46 | 11.9 |
| Adjuvant Chemotherapy | | |
| No | 311 | 80.2 |
| Yes | 77 | 19.8 |
| Emergency Presentation | | |
| No | 345 | 88.9 |
| Yes | 43 | 11.1 |

AFFYMETRIX GENECHIP ® ANALYSIS

| | GBP-1-positive | GBP-1-negative | P value |
|---|---|---|---|
| n | 12 | 12 | |
| Sex ratio (male/female) | 6/6 = 1 | 8*/3 = 2.6 | 0.265 |
| Age median/range (years) | 69.5/47-80 | 63*/46-75 | 0.453 |
| Tumor Site | | | 0.111 |
| Sigmoid colon | | 2 | |
| Rectum | 5 | 8 | |
| Descending colon | | 1 | |
| Splenic flexure | | 1 | |
| Transverse colon | 1 | | |
| Hepatic flexure | 1 | | |
| Ascending colon | 1 | | |
| Cecum | 4 | | |
| Stage (UICC 2002) | | | 0.459 |
| I | 3 | 2 | |
| II | 4 | 2 | |
| III | 5 | 8 | |
| Primary Tumor | | | 0.128 |
| pT1 | 1 | | |
| pT2 | 3 | 3 | |
| pT3 | 8 | 5 | |
| pT4 | | 4 | |
| Regional Lymph Nodes | | | 0.148 |
| pN0 | 7 | 4 | |
| pN1 | 5 | 5 | |
| pN2 | | 3 | |
| Distant Metastasis | | | |
| M0 | 12 | 12 | |
| Histopathological | | | 0.132 |
| Grading | | | |
| G2 | 11 | 8 | |
| G3 | 1 | 4 | |
| Adjuvant chemotherapy | 12/0 | 11/1 | 0.307 |
| (yes/no) | | | |

P value was assessed using Pearson's chi square test.
*Gender and age of one patient was unknown.

TABLE 2
GBP-1 Expression is Highly Significant Associated with
UICC Stage II/pN0-status of Colonic Carcinoma (n = 388).

| | GBP-1 negative | | GBP-1 positive | | |
|---|---|---|---|---|---|
| | n = 264 | | n = 124 | | P value |
| Stage (UICC 2002) | | | | | <0.001 |
| II | 113 | (42.8%) | 80 | (64.6%) | |
| III | 122 | (46.2%) | 37 | (29.8%) | |
| IV | 29 | (11%) | 7 | (5.6%) | |
| Primary Tumor | | | | | 0.411 |
| pT2 | 16 | (6.0%) | 11 | (8.9%) | |
| pT3 | 211 | (79.9%) | 100 | (80.6%) | |
| pT4 | 37 | (14.1%) | 13 | (10.5%) | |
| Regional Lymph Nodes | | | | | <0.001 |
| pN0 | 119 | (45.1%) | 84 | (67.7%) | |
| pN1 | 85 | (32.2%) | 25 | (20.2%) | |
| pN2 | 60 | (22.7%) | 15 | (12.1%) | |
| Histopathological | | | | | 0.264 |
| Grading | | | | | |
| Low grade (G1/G2) | 219 | (83.0%) | 97 | (78.2%) | |
| High grade (G3/G4) | 45 | (17.0%) | 27 | (21.8%) | |
| Extramural Venous | | | | | 0.056 |
| Invasion | | | | | |
| EVI (−) | 226* | (85.6%) | 114* | (91.9%) | |
| EVI (+) | 37* | (14.0%) | 9* | (7.2%) | |

*Extramural venous invasion status of two patients was unknown. P value was determined by Pearson's chi square test.

TABLE 3
Cancer-related 5-year Survival is Highly Significant Increased in
GBP-1-positive Colonic Carcinoma Patients and Indicates a Significantly
Decreased Relative Risk of Cancer-related Death (n = 388).

| | | 5 year cancer | | |
|---|---|---|---|---|
| UNIVARIATE | | related | | |
| ANALYSIS | n | survival (%) | 95% CI | P value |
| All Patients | 388 | 81.1 | 77.2-85.0 | |
| GBP-1 Expression in | | | | <0.001 |
| the Stroma | | | | |
| GBP-1 neg. (−) | 264 | 76.0 | 70.7-81.3 | |
| GBP-1 pos. (+) | 124 | 92.2 | 87.3-97.1 | |
| Stage (UICC 2002) | | | | <0.001 |
| II | 193 | 91.6 | 87.5-95.7 | |
| III | 159 | 74.2 | 67.3-81.1 | |
| IV | 36 | 57.3 | 40.8-73.8 | |
| Primary Tumor | | | | 0.005 |
| pT2 | 27 | 96.2 | 88.8-100 | |
| pT3 | 311 | 82.3 | 78.0-86.6 | |
| pT4 | 50 | 64.8 | 51.3-78.3 | |
| Regional Lymph Nodes | | | | <0.001 |
| pN0 | 203 | 90.0 | 85.7-94.3 | |
| pN1 | 110 | 86.2 | 79.7-92.7 | |
| pN2 | 75 | 49.1 | 37.3-60.9 | |
| Histopathological | | | | 0.134 |
| Grading | | | | |
| Low grade (G1/G2) | 316 | 82.4 | 78.1-86.7 | |
| High grade (G3/G4) | 72 | 75.2 | 65.0-85.4 | |
| Extramural Venous | | | | <0.001 |
| Invasion | | | | |
| EVI (−) | 340* | 85.8 | 82.1-89.5 | |
| EVI (+) | 46* | 47.6 | 32.7-62.5 | |
| Adjuvant Chemotherapy | | | | 0.207 |
| No | 311 | 82.4 | 78.1-86.7 | |
| Yes | 77 | 75.7 | 65.9-85.5 | |
| Emergency Presentation | | | | <0.001 |
| No | 345 | 83.7 | 79.8-87.6 | |
| Yes | 43 | 57.8 | 42.1-73.5 | |
| MULTIVARIATE | | Relative | | |
| ANALYSIS | n | Risk | 95% CI | P value |
| GBP-1 Expression in | | | | |
| the Stroma | | | | |
| GBP-1 negative (−) | 263 | 1.0 | | |
| GBP-1 positive (+) | 123 | 0.5 | 0.3-0.9 | 0.032 |
| Stage (UICC 2002) | | | | |
| Stage II | 193 | 1.0 | | |
| Stage III | 157 | 2.5 | 1.5-4.2 | 0.001 |
| Stage IV | 36 | 4.3 | 2.2-8.3 | <0.001 |
| Extramural Venous | | | | |
| Invasion | | | | |
| EVI (−) | 340* | 1.0 | | |
| EVI (+) | 46* | 2.7 | 1.7-4.4 | <0.001 |
| Emergency Presentation | | | | |
| No | 344 | 1.0 | | |
| Yes | 42 | 2.1 | 1.2-3.7 | 0.008 |

*Extramural venous invasion status of two patients was unknown. Accordingly, the cancer-related 5-year survival of 388 patients and the relative risk of 386 patients, respectively were analyzed. 95% confidence intervals (95%-CI) and p values as determined by univariate analysis (upper) and multivariate analysis (lower) are given in relation to clinical parameters.

TABLE 4
GBP-1-positive Colorectal Carcinomas (n = 12) were Compared with
GBP-1-negative CRCs (n = 12) by Transcriptome Analysis.

| | | | Accession | | |
|---|---|---|---|---|---|
| GENE No. | Fold change | P value | number | Gene | Group |
| 1 | 25.52 | 0 | AF030514.1 | Homo sapiens interferon stimulated T-cell alpha | IFN, CC |
| | | | | chemoattractant (CXCL11) | |
| 2 | 17.74 | 0.004 | D87021 | Homo sapiens immunoglobulin lambda gene locus DNA | IR |
| 3 | 16.79 | 0 | AF002985.1 | Homo sapiens putative alpha chemokine (H174) | CC |
| 4 | 14.36 | 0 | NM_002416.1 | Homo sapiens monokine induced by gamma interferon | IFN, CC |
| | | | | (CXCL9) | |
| 5 | 14.34 | 0 | NM_005601.1 | Homo sapiens natural killer cell group 7 sequence | IR |
| | | | | (NKG7) | |
| 6 | 13.8 | 0.001 | M24669.1 | Human Ig rearranged H-chain V-region mRNA (C-D- | IR |
| | | | | JH6) | |
| 7 | 13.21 | 0.002 | M24668.1 | Human Ig rearranged H-chain V-region mRNA (C-D- | IR |
| | | | | JH4) | |
| 8 | 13.01 | 0 | NM_001565.1 | Homo sapiens small inducible cytokine subfamily B | IFN, CC |
| | | | | (Cys-X-Cys), member 10 (CXCL10) | |
| 9 | 12.8 | 0 | NM_006820.1 | Homo sapiens interferon-induced protein 44-like | IFN |
| | | | | (IFI44L) | |
| 10 | 12.13 | 0.003 | BG482805 | Homo sapiens rearranged gene for kappa | IR |
| | | | | immunoglobulin subgroup V kappa IV | |
| 11 | 12.07 | 0.001 | L34164.1 | Human Ig rearranged mu-chain gene VH3-D2110-JH2 | IR |
| 12 | 10.81 | 0.002 | AV698647 | Homo sapiens immunoglobulin lambda joining 3 | IR |
| 13 | 10.77 | 0 | L14458.1 | Human Ig rearranged kappa-chain gene V-J-region | IR |
| 14 | 10.7 | 0 | NM_006419.1 | Homo sapiens small inducible cytokine B subfamily, | CC |
| | | | | member 13 (SCYB13, CXCL13) | |
| 15 | 10.53 | 0.003 | L23518.1 | Human Ig rearranged gamma-chain, V-DXP1-JH4b | IR |
| 16 | 10.26 | 0.005 | U80139 | Human immunoglobulin heavy chain variable region | IR |
| | | | | (V4-4) gene | |
| 17 | 10.12 | 0.001 | L23516.1 | Human Ig rearranged gamma-chain, V-DXP4-JH6c | IR |
| 18 | 9.84 | 0.001 | AJ408433 | Homo sapiens partial IGKV gene for immunoglobulin | IR |
| | | | | kappa chain variable region, clone 38 | |
| 19 | 9.65 | 0.003 | M24670.1 | Human Ig rearranged H-chain V-region mRNA (C-D- | IR |
| | | | | JH6) | |
| 20 | 9.07 | 0.005 | AF234255.1 | Homo sapiens clone KM36 immunoglobulin light chain | IR |
| | | | | variable region | |
| 21 | 8.92 | 0 | BG540628 | Human active IgK chain from GM 607, V-kappa-2 | IR |
| | | | | region | |
| 22 | 8.88 | 0.007 | D84143.1 | Human immunoglobulin (mAb59) light chain V region | IR |
| 23 | 8.79 | 0.002 | M85256.1 | Homo sapiens immunoglobulin kappa-chain VK-1 | IR |
| | | | | (IgK) | |
| 24 | 8.73 | 0.002 | AJ275408 | Homo sapiens partial IGVH3 gene for immunoglobulin | IR |
| | | | | heavy chain V region, case 1, cell Mo VI 162 | |
| 25 | 8.58 | 0 | M21121 | Human T cell-specific protein (RANTES) | CC |
| 26 | 8.51 | 0.001 | M34455.1 | Human interferon-gamma-inducible indoleamine 2,3- | IFN |
| | | | | dioxygenase (IDO) | |
| 27 | 8.5 | 0.001 | X51887 | Human V108 gene encoding an immunoglobulin kappa | IR |
| | | | | orphon | |
| 28 | 8.07 | 0.004 | AJ275397 | Homo sapiens partial IGVH1 gene for immunoglobulin | IR |
| | | | | heavy chain V region, case 1, cell Mo V 94 | |
| 29 | 7.71 | 0.002 | AB035175 | Homo sapiens IgH VH gene for immunoglobulin heavy | IR |
| | | | | chain | |
| 30 | 7.7 | 0.001 | L14457.1 | Human Ig rearranged kappa-chain gene V-J-region | IR |
| 31 | 7.65 | 0.003 | AF103529.1 | Homo sapiens isolate donor N clone N88K | IR |
| | | | | immunoglobulin kappa light chain variable region | |
| 32 | 7.46 | 0.024 | AF047245.1 | Homo sapiens clone bsmneg3-t7 immunoglobulin | IR |
| | | | | lambda light chain VJ region, (IGL) | |
| 33 | 7.45 | 0.005 | NM_021181.2 | Homo sapiens SLAM family member 7 (SLAMF7) | IR |
| 34 | 7.44 | 0.001 | AJ275469 | Homo sapiens partial IGVH3 gene for immunoglobulin | IR |
| | | | | heavy chain V region, case 2, cell E 172 | |
| 35 | 7.35 | 0.001 | H53689 | Homo sapiens clone ASPBLL54 immunoglobulin | IR |
| | | | | lambda light chain VJ region | |
| 36 | 7.29 | 0.001 | AJ249377.1 | Homo sapiens partial mRNA for human Ig lambda light | IR |
| | | | | chain variable region, clone MB91 | |
| 37 | 7.2 | 0.003 | M16768.1 | Human T-cell receptor gamma chain VJCI-CII-CIII | IR |
| | | | | region | |
| 38 | 7.11 | 0.001 | M85276 | Homo sapiens NKG5 gene | other |
| 39 | 6.92 | 0.009 | M87268.1 | Human IgM VDJ-region | IR |
| 40 | 6.82 | 0.001 | Y13710 | Homo sapiens mRNA for alternative activated | CC |
| | | | | macrophage specific CC chemokine 1 | |
| 41 | 6.73 | 0 | BC002666.1 | Homo sapiens, guanylate binding protein 1, | IFN |
| | | | | interferon-inducible, 67 kD | |
| 42 | 6.73 | 0.001 | AW408194 | Homo sapiens immunoglobulin kappa variable 1-13 | IR |
| 43 | 6.72 | 0 | NM_000579.1 | Homo sapiens chemokine (C-C motif) receptor 5 | CC |
| | | | | (CCR5) | |
| 44 | 6.69 | 0.008 | BF002659 | Myosin-reactive immunoglobulin heavy chain variable | IR |
| | | | | region | |
| 45 | 6.47 | 0 | NM_004335.2 | Homo sapiens bone marrow stromal cell antigen 2 | IR |
| | | | | (BST2) | |
| 46 | 6.43 | 0.005 | AF043583.1 | Homo sapiens clone ASMneg1-b3 immunoglobulin | IR |
| | | | | lambda chain VJ region, (IGL) | |
| 47 | 6.36 | 0 | NM_004585.2 | Homo sapiens retinoic acid receptor responder | other |
| | | | | (tazarotene induced) 3 (RARRES3) | |
| 48 | 6.31 | 0.003 | X79782.1 | H. sapiens (T1.1) mRNA for IG lambda light chain. | IR |
| 49 | 6.22 | 0.004 | X93006.1 | H. sapiens mRNA for IgG lambda light chain V-J-C | IR |
| | | | | region (clone Tgl11) | |
| 50 | 6.19 | 0.002 | NM_006433.2 | Homo sapiens granulysin (GNLY), transcript variant | IR |
| | | | | NKG5 | |
| 51 | 6.17 | 0.001 | AA680302 | Homo sapiens immunoglobulin lambda locus | IR |
| 52 | 6.03 | 0.001 | BG536224 | Human kappa-immunoglobulin germline pseudogene | IR |
| | | | | (Chr22.4) variable region (subgroup V kappa II) | |
| 53 | 5.81 | 0.015 | L23519.1 | Human Ig rearranged gamma-chain, V-DK4-JH4b | IR |
| 54 | 5.7 | 0 | AI984980 | small inducible cytokine subfamily A, member 8 | CC |
| | | | | (monocyte chemotactic protein 2) (MCP-2) | |
| 55 | 5.69 | 0.002 | AB000221.1 | Homo sapiens mRNA for CC chemokine | CC |
| 56 | 5.65 | 0.005 | AJ239383.1 | Homo sapiens mRNA for immunoglobulin heavy chain | IR |
| | | | | variable region, ID 31 | |
| 57 | 5.63 | 0.001 | U92706 | Homo sapiens mRNA for single-chain antibody | IR |
| 58 | 5.6 | 0.002 | AB001733.1 | Homo sapiens mRNA for single-chain antibody | IR |
| 59 | 5.52 | 0 | NM_006144.2 | Homo sapiens granzyme A (granzyme 1, cytotoxic | IR |
| | | | | T-lymphocyte-associated serine esterase 3) GZMA | |
| 60 | 5.45 | 0.003 | AW404894 | Homo sapiens partial IGKV gene for immunoglobulin | IR |
| | | | | kappa chain variable region, clone 30 | |
| 61 | 5.43 | 0.001 | NM_001548.1 | Homo sapiens interferon-induced protein with | IFN |
| | | | | tetratricopeptide repeats 1 (IFIT1) | |
| 62 | 5.42 | 0.001 | NM_000570.1 | Homo sapiens Fc fragment of IgG, low affinity IIIb, | IR |
| | | | | receptor for (CD16) (FCGR3B) | |
| 63 | 5.35 | 0.001 | AF103530.1 | Homo sapiens isolate donor N clone N8K | IR |
| | | | | immunoglobulin kappa light chain variable region | |
| 64 | 5.33 | 0.001 | M20812 | Human kappa-immunoglobulin germline pseudogene | IR |
| | | | | (cos118) variable region (subgroup V kappa I) | |
| 65 | 5.25 | 0 | NM_002535.1 | Homo sapiens 2′-5′-oligoadenylate synthetase 2 | IFN |
| | | | | (OAS2), transcript variant 2 | |
| 66 | 5.08 | 0 | AI337069 | Homo sapiens cDNA clone IMAGE 2009047 | other |
| 67 | 5.04 | 0.001 | M30894.1 | Human T-cell receptor Ti rearranged gamma-chain | IR |
| | | | | mRNA V-J-C region | |
| 68 | 5 | 0.001 | BG340548 | Human rearranged immunoglobulin heavy chain | IR |
| 69 | 4.98 | 0.001 | BG485135 | immunoglobulin kappa variable 3D-15 | IR |
| 70 | 4.98 | 0.001 | AB014341.1 | Homo sapiens mRNA for VEGF single chain antibody | IR |
| 71 | 4.93 | 0.001 | AF043179.1 | Homo sapiens T cell receptor beta chain (TCRBV13S1- | IR |
| | | | | TCRBJ2S1) | |
| 72 | 4.87 | 0.001 | M87790.1 | Human (hybridoma H210) anti-hepatitis A | IR |
| | | | | immunoglobulin lambda chain variable region, | |
| | | | | constant region, complementarity-determining regions | |
| 73 | 4.79 | 0 | AI768628 | Homo sapiens IMAGE clone similar to: chloride | other |
| | | | | intracellular channel 2 | |
| 74 | 4.69 | 0.001 | M27487.1 | Homo sapiens MHC class II DPw3-alpha-1 chain | IR |
| 75 | 4.54 | 0.013 | L14456.1 | Human Ig rearranged mu-chain gene V-N-D-N-J-region | IR |
| 76 | 4.51 | 0 | NM_006332.1 | Homo sapiens interferon, gamma-inducible protein 30 | IFN |
| | | | | (IFI30) | |
| 77 | 4.47 | 0 | NM_017523.1 | Homo sapiens XIAP associated factor-1 (BIRC4BP) | other |
| 78 | 4.41 | 0.007 | BG397856 | major histocompatibility complex, class II, DQ alpha 1 | IR |
| 79 | 4.4 | 0 | BC002704.1 | Homo sapiens, Similar to signal transducer and activator | IFN |
| | | | | of transcription 1, 91 kd | |
| 80 | 4.39 | 0.001 | NM_022873.1 | Homo sapiens interferon, alpha-inducible protein (clone | IFN |
| | | | | IFI-6-16) (G1P3), transcript variant 3 | |
| 81 | 4.36 | 0 | NM_002462.1 | Homo sapiens myxovirus (influenza) resistance 1, | IFN |
| | | | | homolog of murine (interferon-inducible protein p78) | |
| | | | | (MX1) | |
| 82 | 4.33 | 0 | M87789.1 | Human (hybridoma H210) anti-hepatitis A IgG variable | IR |
| | | | | region, constant region, complementarity-determining | |
| | | | | regions | |
| 83 | 4.31 | 0.002 | X57812.1 | Human rearranged immunoglobulin lambda light chain | IR |
| 84 | 4.29 | 0 | NM_006398.1 | Homo sapiens diubiquitin (UBD) | other |
| 85 | 4.27 | 0 | NM_002838.1 | Homo sapiens protein tyrosine phosphatase, receptor | other |
| | | | | type, C (PTPRC) | |
| 86 | 4.27 | 0.001 | NM_001803.1 | Homo sapiens CD52 antigen (CAMPATH-1 antigen) (CD52) | IR |
| 87 | 4.25 | 0 | NM_001775.1 | Homo sapiens CD38 antigen (p45) (CD38) | IR |
| 88 | 4.25 | 0.002 | M80927.1 | Human glycoprotein mRNA | other |
| 89 | 4.21 | 0.007 | NM_006498.1 | Homo sapiens lectin, galactoside-binding, soluble, 2 | IR |
| | | | | (galectin 2) (LGALS2) | |
| 90 | 4.19 | 0 | NM_005101.1 | Homo sapiens interferon-alpha inducile (clone IFI-ISK) | IFN |
| | | | | (G1P2) | |
| 91 | 4.19 | 0 | NM_006417.1 | Homo sapiens interferon-induced, protein 44 (IFI 44) | IFN |
| 92 | 4.17 | 0.001 | BC000879.1 | Homo sapiens, Similar to kynureninase (L-kynurenine | other |
| | | | | hydrolase), clone MGC:5080 | |
| 93 | 4.14 | 0.001 | M60334.1 | Human MHC class II HLA-DR-alpha | IR |
| 94 | 4.13 | 0.003 | NM_004503.1 | Homo sapiens homeo box C6 (HOXC6) | other |
| 95 | 4.09 | 0.001 | NM_012307.1 | Homo sapiens erythrocyte membrane protein band 4.1- | other |
| | | | | like 3 (EPB41L3) | |
| 96 | 4.08 | 0 | NM_004244.1 | Homo sapiens CD163 antigen (CD163) | IR |
| 97 | 4.08 | 0 | NM_002201.2 | Homo sapiens interferon stimulated gene (20 kD) (ISG20) | IFN |
| 98 | 4.07 | 0 | AI809341 | IMAGE clone similar to: protein tyrosine phosphatase, | other |
| | | | | receptor type, C (PTPRC) | |
| 99 | 4.07 | 0.002 | M60333.1 | Human MHC class II HLA-DRA | IFN |
| 100 | 4.05 | 0.003 | NM_001623.2 | Human allograft-inflammatory factor-1 (AIF-1) | IFN |
| 101 | 4.04 | 0 | NM_017631.1 | hypothetical protein FLJ20035 | other |
| 102 | 4.02 | 0 | NM_002121.1 | Homo sapiens major histocompatibility complex, class | IR |
| | | | | II, DPbeta 1 | |
| 103 | 4.02 | 0.002 | AL022324 | Human DNA sequence from clone CTA-246H3 on | IR |
| | | | | chromosome 22 Contains the gene for IGLL1 | |
| | | | | (immunoglobulin lambda-like polypeptide 1, pre-B-cell | |
| | | | | specific) | |
| 104 | 4.01 | 0.015 | M17955.1 | Human MHC class II HLA-DQ-beta | IR |
| 105 | | | Gi:48146240 | Homo sapiens, guanylate binding protein 2, | |
| 106 | | | Gi:24308156 | Homo sapiens, guanylate binding protein 3, | |
| 107 | | | Gi:15558942 | Homo sapiens, guanylate binding protein 4, | |
| 108 | | | Gi 31377630 | Homo sapiens, guanylate binding protein 5, | |

Genes estimated to be significantly increased in GBP-1-positive CRC are given in the table by fold change increase. Genes were functionally grouped into IFN-induced genes (IFN), chemokines (CC), immune reaction-associated genes (IR) and others. P value was assessed by Mann-Whitney-U-test. Gene names and the corresponding gene bank number are given. The three antiangiogenic chemokines and GBP-1 are shaded.

TABLE 5
Genes Downregulated in GBP-1-positive CRC

| | Average | Average | | p | Accession | |
|---|---|---|---|---|---|---|
| | GBP-1- | GBP-1- | | value of | number | |
| GENE | positive | negative | Fold | differential | in | |
| No. | CRC | CRC | increase | expression | GENBANK ® | Description |
| 109 | 79.12 | 1470.02 | 18.58 | 0.008 | NM_000439.2 | Homo sapiens proprotein convertase |
| | | | | | | subtilisinkexin type 1 (PCSK1) |
| 110 | 45.22 | 472.22 | 10.44 | 0.006 | NM_004626.1 | Homo sapiens wingless-type MMTV |
| | | | | | | integration site family, member 11 |
| | | | | | | (WINT11) |
| 111 | 175.88 | 795.85 | 4.52 | 0.038 | NM_001853.1 | Homo sapiens collagen, type IX, alpha 3 |
| | | | | | | (COL9A3) |
| 112 | 309.95 | 1387.91 | 4.48 | 0.033 | NM_007197.1 | Homo sapiens frizzled (Drosophila) |
| | | | | | | homolog 10 (FZD10) |
| 113 | 186.97 | 722.4 | 3.86 | 0.05 | NM_007191.1 | Homo sapiens Wnt inhibitory factor-1 |
| | | | | | | (WIF-1) |
| 114 | 94.52 | 348.81 | 3.69 | 0.003 | AF202063.1 | Homo sapiens fibroblast growth factor |
| | | | | | | receptor 4, soluble-form splice variant |
| | | | | | | (FGFR4) |
| 115 | 1435.76 | 5248.49 | 3.66 | 0.008 | NM_001823.1 | Homo sapiens creatine kittase, brain (CKI3) |
| 116 | 130,63 | 447.83 | 3.43 | 0.021 | NM_004796.1 | Homo sapiens rietirexin 3 (NRXN3) |
| 117 | 159.13 | 526.83 | 3.31 | 0.002 | NM_004636.1 | Homo sapiens sema domain, |
| | | | | | | immunoglobulin domain (1g), short basic |
| | | | | | | domain, secreted, (sernaphorin) 3B |
| | | | | | | (SEMA3B) |
| 118 | 204.43 | 663.17 | 3.24 | 0.001 | NM_012410.1 | Homo sapiens type 1 transmembrane |
| | | | | | | receptor (seizure-related protein) (PSK- |
| | | | | | | 1) |
| 119 | 1078.19 | 3477.69 | 3.23 | 0.043 | NM_005588.1 | Homo sapiens meprin A, alpha (PABA |
| | | | | | | peptide hydrolase) (MEPIA) |
| 120 | 285.67 | 837.78 | 2.93 | 0.043 | NM_006198.1 | Homo sapiens Purkinje cell protein 4 |
| | | | | | | (PCP4) |
| 121 | 183.81 | 534.82 | 2.91 | 0.021 | AF195953 | Homo sapiens membrane-bound |
| | | | | | | aminopeptidase P (XNPEP2) |
| 122 | 112.07 | 322.61 | 2.88 | 0.033 | AW770748 | imprinted in Prader-Willi syndrome |
| 123 | 332.18 | 898.32 | 2.7 | 0.002 | AB002360.1 | Human mRNA for KIAA0362 gene |
| 124 | 5098.08 | 13469.6 | 2.64 | 0.033 | D13889.1 | Human mRNA fix Id-1H |
| 125 | 1745.44 | 4395.77 | 2.52 | 0.003 | NM_003212.1 | Homo sapiens teratocarcinoma-derived |
| | | | | | | growth factor 1 (TDGF1) |
| 126 | 137.29 | 344.38 | 2.51 | 0.021 | NM_001808.1 | Homo sapiens carboxyl ester lipase-like |
| | | | | | | (bile salt-stimulated lipase-like) (CELL) |
| 127 | 269.58 | 670.96 | 2.49 | 0 | NM_017797.1 | Homo sapiens BTB (POZ) domain |
| | | | | | | containing 2 (BTBD2) |
| 128 | 472.86 | 1153.52 | 2.44 | 0.004 | NM 015392.1 | Homo sapiens neural proliferation, |
| | | | | | | differentiation and control, 1 (NPDC1) |
| 129 | 156.47 | 372.88 | 2.38 | 0.009 | AL531533 | branched chain keto acid dehydrogenase E1, |
| | | | | | | beta polypeptide (maple syrup urine |
| | | | | | | disease) |
| 130 | 864.83 | 2043.48 | 2.36 | 0.043 | NM_001926.2 | Homo sapiens defensin, alpha 6, Paneth |
| | | | | | | cell-specific (DEFA6) |
| 131 | 3010.33 | 6976.21 | 2.32 | 0.002 | NM_018487.1 | Homo sapiens hepatocellular carcinoma- |
| | | | | | | associated antigen 112 (HCA112) |
| 132 | 138.36 | 319.83 | 2.31 | 0.001 | NM_000724.1 | Homo sapiens calcium channel, voltage- |
| | | | | | | dependent, beta 2 subunit (CACNB2) |
| 133 | 176.45 | 406.52 | 2.3 | 0.008 | NM_021924.1 | Homo sapiens mucin and cadherin-like |
| | | | | | | (M UCDHL) |
| 134 | 742.42 | 1703.29 | 2.29 | 0.007 | NM_002591.1 | Homo sapiens phosphoenolpyruvate |
| | | | | | | carboxykinase 1 (soluble) (PCK1) |
| 135 | 987.26 | 2255.8 | 2.28 | 0.006 | AL049593 | phosphoinositide-specific phospholipase C- |
| | | | | | | beta 1 /DEF |
| 136 | 397.75 | 902.54 | 2.27 | 0.018 | NM_025081.1 | Homo sapiens KIAA1305 protein |
| | | | | | | (KIAA1305) |
| 137 | 230.82 | 521.74 | 2.26 | 0.021 | NM_013358.1 | Homo sapiens peptidylarginine deiminase |
| | | | | | | type I (hPAD-colony10) |
| 138 | 2061.12 | 4619.07 | 2.24 | 0.003 | L20817.1 | Homo sapiens tyrosine protein kinase |
| | | | | | | (CAK) gene |
| 139 | 257.46 | 576.21 | 2.24 | 0.015 | NM_000015.1 | Homo sapiens N-acetyltransferase 2 |
| | | | | | | (arylamine N-acetyltransferase) (NAT2) |
| 140 | 176.29 | 393.54 | 2.23 | 0.038 | X17406.1 | Human mRNA for cartilage specific |
| | | | | | | proteoglycan |
| 141 | 169.29 | 376.37 | 2.22 | 0.021 | NM_005060.1 | Homo sapiens RAR-related orphan receptor |
| | | | | | | C (RORC) |
| 142 | 249.42 | 548.12 | 2.2 | 0.009 | NM_016202.1 | Homo sapiens LDL induced EC protein |
| | | | | | | (LOC51157) |
| 143 | 363.79 | 788.76 | 2.17 | 0.009 | U35622.2 | Homo sapiens EWS proteinEIA enhancer |
| | | | | | | binding protein chimera |
| 144 | 583.47 | 1257.57 | 2.16 | 0.002 | AB038783.1 | Homo sapiens MUC3B mRNA for intestinal |
| | | | | | | mucin |
| 145 | 239.74 | 506.5 | 2.11 | 0.001 | NM_004658.1 | Homo sapiens RAS protein activator like 1 |
| | | | | | | (GAP1 like) (RASAL1) |
| 146 | 390.65 | 822.6 | 2.11 | 0.038 | NM_005975.1 | Homo sapiens PTK6 protein tyrosine kinase |
| | | | | | | 6 (PTK6) |
| 147 | 144.03 | 302.12 | 2.1 | 0.038 | NM_000504.2 | Homo sapiens coagulation factor X (F10) |
| 148 | 523.33 | 1094.1 | 2.09 | 0.008 | NM_000196.1 | Homo sapiens hydroxysteroid (11-beta) |
| | | | | | | dehydrogenase 2 (HSD11B2) |
| 149 | 2572.06 | 5352.47 | 2.08 | 0.008 | NM_001038.1 | Homo sapiens sodium channel, nonvoltage- |
| | | | | | | gated 1 alpha (SCNN1A) |
| 150 | 2141.68 | 4420.33 | 2.06 | 0.002 | NM_001954.2 | Homo sapiens discoidin domain receptor |
| | | | | | | family, member 1 (DDR1), transcript |
| | | | | | | variant 2 |
| 151 | 2173.38 | 4478.25 | 2.06 | 0.021 | NM_003915.1 | Homo sapiens copine I (CPNE1) |
| 152 | 573.38 | 1167.21 | 2.04 | 0.001 | U51096.1 | Human homeobox protein Cdx2 |
| 153 | 8537.94 | 17329.82 | 2.03 | 0.005 | BE542815 | general transcription factor IIIA |
| 154 | 456.18 | 925.45 | 2.03 | 0.038 | NM_004624.1 | Homo sapiens vasoactive intestinal peptide |
| | | | | | | receptor 1 (VIPR1) |
| 155 | 691.82 | 1399.03 | 2.02 | 0.043 | NM_002705.1 | Homo sapiens periplakin (PPL) |
| 156 | 217.06 | 437.27 | 2.01 | 0.013 | NM_016339.1 | 1-lomo sapiens Link guanine nucleotide |
| | | | | | | exchange factor II (LOC51195) |
| 157 | 892.73 | 1783.97 | 2 | 0.011 | NM_005766.1 | Homo sapiens FERM, RhoGEF (ARHGEF) |
| | | | | | | and pleckstrin domain protein 1 |
| | | | | | | (chondrocyte-derived) (FARP1) |

TABLE 6
The Association of GBP-1 Expression with UICC II Stage/pN0 Status is Independent
of the Absolute Number of GBP-1-positive Cells and GBP-1 Expression Level.

| | GBP-1: Number of Cells | | | | |
|---|---|---|---|---|---|
| | 0 | 1 | 2 | 3 | P value |
| UICC stage | | | | | |
| II | 122 (43.4%) | 37 (61.7%) | 28 (60.9%) | 20 (80%) | 0.001 |
| III | 129 (45.9%) | 22 (36.7%) | 14 (30.4%) | 3 (12%) | |
| IV | 30 (10.7%) | 1 (1.7%) | 4 (8.7%) | 2 (8%) | |
| Pathologic | | | | | |
| Lymph Node | | | | | |
| Status | | | | | |
| pN0 | 128 (45.6%) | 38 (63.3%) | 30 (65.2%) | 21 (84%) | 0.002 |
| pN1 | 91 (32.4%) | 13 (21.7%) | 10 (21.7%) | 3 (12%) | |
| pN2 | 62 (22.1%) | 9 (15%) | 6 (13%) | 1 (4%) | |

| | GBP-1: Expression Level | | | | |
|---|---|---|---|---|---|
| | − | + | ++ | +++ | P value |
| UICC stage | | | | | |
| II | 122 (43.4%) | 39 (62.9%) | 39 (66.1%) | 7 (70%) | 0.002 |
| III | 129 (45.9%) | 20 (32.3%) | 18 (30.5%) | 1 (10%) | |
| IV | 30 (10.7%) | 3 (4.8%) | 2 (3.4%) | 2 (20%) | |
| Pathologic | | | | | |
| Lymph Node | | | | | |
| Status | | | | | |
| pN0 | 128 (45.6%) | 41 (66.1%) | 39 (66.1%) | 9 (90%) | 0.002 |
| pN1 | 91 (32.4%) | 14 (22.6%) | 11 (18.6%) | 1 (10%) | |
| pN2 | 62 (22.1%) | 7 (11.3%) | 9 (15.3%) | — | |

CRC tissue arrays were immunohistochemically stained for GBP-1. Numbers of positive cells (0, negative; 1, <50%; 2, ~50%; 3, >50%) and expression levels (−, negative; +, weak; ++, middle; +++, high) were determined. P values given were assessed by Pearsons's chi square test.

### Sequences:

```
CXCL9 (GENE No. 4):
NUCLEIC ACID SEQUENCE
(SEQ ID NO: 1)
      1 atccaataca ggagtgactt ggaactccat tctatcacta tgaagaaaag tggtgttctt
    61 ttcctcttgg gcatcatctt gctggttctg attggagtgc aaggaacccc agtagtgaga
  121 aagggtcgct gttcctgcat cagcaccaac caagggacta tccacctaca atacttgaaa
  181 gaccttaaac aatttgcccc aagcccttcc tgcgagaaaa ttgaaatcat tgctacactg
  241 aagaatggag ttcaaacatg tctaaaccca gattcagcag atgtgaagga actgattaaa
  301 aagtgggaga aacaggtcag ccaaaagaaa aagcaaaaga atgggaaaaa acatcaaaaa
  361 aagaaagtcc tgaaagttcg aaaatctcaa cgttctcgtc aaaagaagac tacataagag
  421 accacttcac caataagtat tctgtgttaa aaatgttcta ttttaattat accgctatca
  481 ttccaaagga ggatggcata taatacaaag gcttattdat ttgactagaa aatttaaaac
  541 attactctga aattgtaact aaagttagaa agttgatttt aagaatccaa acgttaagaa
  601 ttgttaaagg ctatgattgt ctttgttctt ctaccaccca ccagttgaat ttcatcatgc
  661 ttaaggccat gattttagca atacccatgt ctacacagat gttcacccaa ccacatccca
  721 ctcacaacag ctgcctggaa gagcagccct aggcttccac gtactgcagc ctccagagag
  781 tatctgaggc acatgtcagc aagtcctaag cctgttagca tgctggtgag ccaagcagtt
  841 tgaaattgag ctggacctca ccaagctgct gtggccatca acctctgtat ttgaatcagc
  901 ctacaggcct cacacacaat gtgtctgaga gattcatgct gattgttatt gggtatcacc
  961 actggagatc accagtgtgt ggctttcaga gcctcctttc tggctttgga agccatgtga
1021 ttccatcttg cccgctcagg ctgaccactt tatttctttt tgttcccctt tgcttcattc
1081 aagtcagctc ttctccatcc taccacaatg cagtgccttt cttctctcca gtgcacctgt
1141 catatgctct gatttatctg agtcaactcc tttctcatct tgtccccaac accccacaga
1201 agtgctttct tctcccaatt catcctcact cagtccagct tagttcaagt cctgcctctt
1261 aaataaacct ttttggacac acaaattatc ttaaaactcc tgtttcactt ggttcagtac
1321 cacatgggtg aacactcaat ggttaactaa ttcttgggtg tttatcctat ctctccaacc
1381 agattgtcag ctccttgagg gcaagagcca cagtatattt ccctgtttct tccacagtgc
1441 ctaataatac tgtggaacta ggttttaata attttttaat tgatgttgtt atgggcagga
1501 tggcaaccag accattgtct cagagcaggt gctggctctt tcctggctac tccatgttgg
1561 ctagcctctg gtaacctctt acttattatc ttcaggacac tcactacagg gaccagggat
1621 gatgcaacat ccttgtcttt ttatgacagg atgtttgctc agcttctcca acaataagaa
1681 gcacgtggta aaacacttgc ggatattctg gactgttttt aaaaaatata cagtttaccg
1741 aaaatcatat aatcttacaa tgaaaaggac tttatagatc agccagtgac caaccttttc
1801 ccaaccatac aaaaattcct tttcccgaag gaaaagggct ttctcaataa gcctcagctt
1861 tctaagatct aacaagatag ccaccgagat ccttatcgaa actcatttta ggcaaatatg
1921 agttttattg tccgtttact tgtttcagag tttgtattgt gattatcaat taccacacca
1981 tctcccatga agaaagggaa cggtgaagta ctaagcgcta gaggaagcag ccaagtcggt
2041 tagtggaagc atgattggtg cccagttagc ctctgcagga tgtggaaacc tccttccagg
2101 ggaggttcag tgaattgtgt aggagaggtt gtctgtggcc agaatttaaa cctatactca
2161 ctttcccaaa ttgaatcact gctcacactg ctgatgattt agagtgctgt ccggtggaga
2221 tcccacccga acgtcttatc taatcatgaa actccctagt tccttcatgt aacttccctg
2281 aaaaatctaa gtgtttcata aatttgagag tctgtgaccc acttaccttg catctcacag
2341 gtagacagta tataactaac aaccaaagac tacatattgt cactgacaca cacgttataa
2401 tcatttatca tatatataca tacatgcata cactctcaaa gcaaataatt tttcacttca
2461 aaacagtatt gacttgtata ccttgtaatt tgaaatattt tctttgttaa aatagaatgg
2521 tatcaataaa tagaccatta atcag
AMINO ACID SEQUENCE
(SEQ ID NO: 2)
MKKSGVLFLLGIILLVLIGVQGTPVVRKGRCSCISTNQGTIHLQSLKDLKQFAPSPSCEKIEII
ATLKNGVQTCLNPDSADVKELIKKWEKQVSQKKKQKNGKKHQKKKVLKVRKSQRSRQKKTT
CXCL10 (GENE No. 8):
NUCLEIC ACID SEQUENCE
(SEQ ID NO: 3)
      1 gagacattcc tcaattgctt agacatattc tgagcctaca gcagaggaac ctccagtctc
    61 agcaccatga atcaaactgc gattctgatt tgctgcctta tctttctgac tctaagtggc
  121 attcaaggag tacctctctc tagaaccgta cgctgtacct gcatcagcat tagtaatcaa
  181 cctgttaatc caaggtcttt agaaaaactt gaaattattc ctgcaagcca attttgtcca
  241 cgtgttgaga tcattgctac aatgaaaaag aagggtgaga agagatgtct gaatccagaa
  301 tcgaaggcca tcaagaattt actgaaagca gttagcaagg aaatgtctda aagatctcct
  361 taaaaccaga ggggagcaaa atcgatgcag tgcttccaag gatggaccac acagaggctg
  421 cctctcccat cacttcccta catggagtat atgtcaagcc ataattgttc ttagtttgca
  481 gttacactaa aaggtgacca atgatggtca ccaaatcagc tgctactact cctgtaggaa
  541 ggttaatgtt catcatccta agctattcag taataactct accctggcac tataatgtaa
  601 gctctactga ggtgctatgt tcttagtgga tgttctgacc ctgcttcaaa tatttccctc
  661 acctttccca tcttccaagg gtactaagga atctttctgc tttggggttt atcagaattc
  721 tcagaatctc aaataactaa aaggtatgca atcaaatctg ctttttaaag aatgctcttt
  781 acttcatgga cttccactgc catcctccca aggggcccaa attctttcag tggctaccta
  841 catacaattc caaacacata caggaaggta gaaatatctg aaaatgtatg tgtaagtatt
  901 cttatttaat gaaagactgt acaaagtata agtcttagat gtatatattt cctatattgt
  961 tttcagtgta catggaataa catgtaatta agtactatgt atcaatgagt aacaggaaaa
1021 ttttaaaaat acagatagat atatgctctg catgttacat aagataaatg tgctgaatgg
1081 ttttcaaata aaaatgaggt actctcctgg aaatattaag aaagactatc taaatgttga
1141 aagatcaaaa ggttactaaa gtaattataa ct
ANIM ACID SEQUENCE
(SEQ ID NO: 4)
MNQTAILICCLIFLTLSGIQGVPLSRTVRCTCISISNQPVNPRSLEKLEIIPASQFCPRVEIIA
TMKKKGEKRCLNPESKAIKNLLKAVSKEMSKRSP
CXCL11 (GENE No. 1):
NUCLEIC ACID SEQUENCE
(SEQ ID NO: 5)
      1 ttcctttcat gttcagcatt tctactcctt ccaagaagag cagcaaagct gaagtagcag
    61 caacagcacc agcagcaaca gcaaaaaaca aacatgagtg tgaagggcat ggctatagcc
  121 ttggctgtga tattgtgtgc tacagttgtt caaggcttcc ccatgttcaa aagaggacgc
  181 tgtctttgca taggccctgg ggtaaaagca gtgaaagtgg cagatattga gaaagcctcc
  241 ataatgtacc caagtaacaa ctgtgacaaa atagaagtga ttattaccct gaaagaaaat
  301 aaaggacaac gatgcctaaa tcccaaatcg aagcaagcaa ggcttataat caaaaaagtt
  361 gaaagaaaga atttttaaaa atatcaaaac atatgaagtc ctggaaaagg gcatctgaaa
  421 aacctagaac aagtttaact gtgactactg aaatgacaag aattctacag taggaaactg
  441 agacttttct atggttttgt gactttcaac ttttgtacag ttatgtgaag gatgaaaggt
  541 gggtgaaagg accaaaaaca gaaatacagt cttcctgaat gaatgacaat cagaattcca
  601 ctgcccaaag gagtccagca attaaatgga tttctaggaa aagctacctt aagaaaggct
  661 ggttaccatc ggagtttaca aagtgctttc acgttcttac ttgttgtatt atacattcat
  721 gcatttctag gctagagaac cttctagatt tgatgcttac aactattctg ttgtgactat
  781 gagaacattt ctgtatctag aagttatctg tctgtattga tctttatgct atattactat
  841 ctgtggttac agtggagaca ttgacattat tactggagtc aagcccttat aagtcaaaag
  901 catctatgtg tcgtaaagca ttcctcaaac attttttcat gcaaatacac acttctttcc
  961 ccaaatatca tgtagcacat caatatgtag ggaaacattc ttatgcatca tttggtttgt
1021 tttataacca attcattaaa tgtaattcat aaaatgtact atgaaaaaaa ttatacgcta
1081 tgggatactg gcaacagtgc acatatttca taaccaaatt agcagcaccg gtcttaattt
1141 gatgtttttc aacttttatt cattgagatg ttttgaagca attaggatat gtgtgtttac
1201 tgtacttttt gttttgatcc gtttgtataa atgatagcaa tatcttggac acatttgaaa
1261 tacaaaatgt ttttgtctac caaagaaaaa tgttgaaaaa taagcaaatg tatacctagc
1321 aatcactttt actttttgta attctgtctc ttagaaaaat acataatcta atcaatttct
1381 ttgttcatgc ctatatactg taaaatttag gtatactcaa gactagttta aagaatcaaa
1441 gtcatttttt tctctaataa actaccacaa cctttctttt ttaaaaaaaa aaa
AMINO ACID SEQUENCE
(SEQ ID NO: 6)
MSVKGMAIALAVILCATVVQGFPMFKRGRCLCIGPGVKAVKVADIEKASIMYPSNNCDKIEVII
TLKENKGQRCLNPKSKQARLIIKKVERKNNF
GBP-1 (GENE No. 41):
NUCLEIC ACID SEQUENCE
(SEQ ID NO: 7)
      1 ggacatggca tcagagatcc acatgacagg cccaatgtgc ctcattgaga acactaatgg
    61 gcgactgatg gcgaatccag aagctctgaa gatcctttct gccattacac agcctatggt
  121 ggtggtggca attgtgggcc tctaccgcac aggcaaatcc tacctgatga acaagctggc
  181 tggaaagaaa aagggcttct ctctgggctc cacggtgcag tctcacacta aaggaatctg
  241 gatgtggtgt gtgccccacc ccaagaagcc aggccacatc ctagttctgc tggacaccga
  301 gggtctggga gatgtagaga agggtgacaa ccagaatgac tcctggatct tcgccctggc
  361 cgtcctcctg agcagcacct tcgtgtacaa tagcatagga accdtcaacc agcaggctat
  421 ggaccaactg tactatatga cagagctgac acatagaatc cgatcaaaat cctcacctga
  481 tgagaatgag aatgaggttg aggattcagc tgactttgtg agcttcttcc cagactttgt
  541 gtggacactg agagatttct ccctggactt ggaagcagat ggacaacccc tcacaccaga
  601 tgagtacctg acatactccc tgaagctgaa gaaaggtacc agtcaaaaag atgaaacttt
  661 taacctgccc agactctgta tccggaaatt cttcccaaag aaaaaatgct ttgtctttga
  721 tcggcccgtt caccgcagga agcttgccca gctcgagaaa ctacaagatg aagagctgga
  781 ccccgaattt gtgcaacaag tagcagactt ctgttcctac atctttagta attccaaaac
  841 taaaactctt tcaggaggca tccaggtcaa cgggcctcgt ctagagagcc tggtgctgac
  901 ctacgtcaat gccatcagca gtggggatct gccgtgcatg gagaacgcag tcctggcctt
  961 ggcccagata gagaactcag ctgcagtgca aaaggctatt gcccactatg aacagcagat
1021 gggccagaag gtgcagctgc ccacagaaag cctccaggag ctgctggacc tgcacaggga
1081 cagtgagaga gaggccattg aagtcttcat caggagttcc ttcaaagatg tggaccatct
1141 atttcaaaag gagttagcgg cccagctaga aaaaaagcgg gatgactttt gtaaacagaa
1201 tcaggaagca tcatcagatc gttgctcagc tttacttcag gtcattttca gtcctctaga
1261 agaagaagtg aaggcgggaa tttattcgaa accagggggc tatcgtctct ttgttcagaa
1321 gctacaagac ctgaagaaaa agtactatga ggaaccgagg aaggggatac aggctgaaga
1381 gattctgcag acatacttga aatccaagga gtctatgact gatgcaattc tccagacaga
1441 ccagactctc acagaaaaag aaaaggagat tgaagtggaa cgtgtgaaag ctgagtctgc
1501 acaggcttca gcaaaaatgt tgcaggaaat gcaaagaaag aatgagcaga tgatggaaca
1561 gaaggagagg agttatcagg aacacttgaa acaactgact gagaagatgg agaacgacag
1621 ggtccagttg ctgaaagagc aagagaggac cctcgctctt aaacttcagg aacaggagca
1681 actactaaaa gagggatttc aaaaagaaag cagaataatg aaaaatgaga tacaggatct
1741 ccagacgaaa atgagacgac gaaaggcatg taccataagc taaagaccag agacttcctg
1801 tca
AMINO ACID SEQUENCE
(SEQ ID NO: 8)
MASEIHMTGPMCLIENTNGRLMANPEALKILSAITQPMVVVAIVGLYRTGKSYLMNKLAGKKKG
FSLGSTVQSHTKGIWMWCVPHPKKPGHILVLLDTEGLGDVEKGDNQNDSWIFALAVLLSSTFVY
NSIGTINQQAMDQLYYVTELTHRIRSKSSPDENENEVEDSADFVSFFPDFVWTLRDFSLDLEAD
GQPLTPDEYLTYSLKLKKGTSQKDETFNLPRLCIRKFFPKKKCFVFDRPVHRRKLAQLEKLQDE
ELDPEFVQQVADFCSYIFSNSKTKTLSGGIQVNGPRLESLVLTYVNAISSGDLPCMENAVLALA
QIENSAAVQKAIAHYEQQMGQKVQLPTESLQELLDLHRDSEREAIEVFIRSSFKDVDHLFQKEL
AAQLEKKRDDFCKQNQEASSDRCSALLQVIFSPLEEEVKAGIYSKPGGYRLFVQKLQDLKKKYY
EEPRKGIQAEEILQTYLKSKESMTDAILQTDQTLTEKEKEIEVERVKAESAQASAKMLQEMQRK
NEQMMEQKERSYQEHLKQLTEKMENDRVQLLKEQERTLALKLQEQEQLLKEGFQKESRIMKNEI
QDLQTKMRRRKACTIS
GBP-2 (GENE No. 105):
NUCLEIC ACID SEQUENCE
(SEQ ID NO: 9)
      1 atggctccag agatcaactt gccgggccca atgagcctca ttgataacac taaagggcag
    61 ctggtggtga atccagaagc tctgaagatc ctatctgcaa ttacgcagcc tgtggtggtg
  121 gtggcgattg tgggcctcta tcgcacaggc aaatcctacc tgatgaacaa gctggctggg
  181 aagaaaaacg gcttctctct aggctccaca gtgaagtctc acaccaaggg aatctggatg
  241 tggtgtgtgc ctcatcccaa gaagccagaa cacaccctag ttctgctcga cactgagggc
  301 ctgggagata tagagaaggg tgacaatgag aatgactcct ggatctttgc cttggccatc
  361 ctcctgagca gcaccttcgt gtacaatagc atgggaacca tcaaccagca gg
  421 caacttcact atgtgacaga gctgacagat cgaatcaagg caaactcctc acctggtaac
  481 aattctgtag acgactcagc tgactttgtg agcttttttc cagcatttgt gtggactctc
  541 agagatttca ccctggaact ggaagtagat ggagaaccca tcactgctga tgactacttg
  601 gagctttcgc taaagctaag aaaaggtact gataagaaaa gtaaaagctt taatgatcct
  661 cggttgtgca tccgaaagtt cttccccaag aggaagtgct tcgtcttcga ttggcccgct
  721 cctaagaagt accttgctca cctagagcag ctaaaggagg aagagctgaa ccctgatttc
  781 atagaacaag ttgcagaatt ttgttcctac atcctcagcc attccaatgt caagactctt
  841 tcaggtggca ttgcagtcaa tgggcctcgt ctagagagcc tggtgctgac ctacgtcaat
  901 gccatcggca gtggggatct accctgcatg gagaacgcag tcctggcctt ggcccagata
  961 gagaactcag ccgcagtgga aaaggctatt gcccactatg aacagcagat gggccagaag
1021 gtgcagctgc ccacggaaac cctccaggag ctgctggacc tgcacaggga cagtgagaga
1081 gaggccattg aagtcttcat gaagaactct ttcaaggatg tggaccaaat gttccagagg
1141 aaattagggg cccagttgga agcaaggcga gatgactttt gtaagcagaa ttccaaagca
1201 tcatcagatt gttgcatggc tttacttcag gatatatttg gccctttaga agaagatgtc
1261 aagcagggaa cattttctaa accaggaggt taccgtctct ttactcagaa gctgcaggag
1321 ctgaagaata agtactacca ggtgccaagg aaggggatac aggccaaaga ggtgctgaaa
1381 aaatatttgg agtccaagga ggatgtggct gatgcacttc tacagactga tcagtcactc
1441 tcagaaaagg aaaaagcgat tgaagtggaa cgtataaagg ctgaatctgc agaagctgca
1501 aagaaaatgt tggaggaaat acaaaagaag aatgaggaga tgatggaaca gaaagagaag
1561 agttatcagg aacatgtgaa acaattgact gagaagatgg agagggacag ggcccagtta
1621 atggcagagc aagagaagac cctcgctctt aaacttcagg aacaggaacg ccttctcaag
1681 gagggattcg agaatgagag caagagactt caaaaagaca tatgggatat ccagatgaga
1741 agcaaatcat tggagccaat atgtaacata ctttaa
AMINO ACID SEQUENCE
(SEQ ID NO: 10)
MAPEINLPGPMSLIDNTKGQLVVNPEALKILSAITQPVVVVAIVGLYRTGKSYLMNKLAGKKNG
FSLGSTVKSHTKGIWMWCVPHPKKPEHTLVLLDTEGLGDIEKGDNENDSWIFALAILLSSTFVY
NSMGTINQQAMDQLHYVTELTDRIKANSSPGNNSVDDSADFVSFFPAFVWTLRDFTLELEVDGE
PITADDYLELSLKLRKGTDKKSKSFNDPRLCIRKFFPKRKCFVFDWPAPKKYLAHLEQLKEEEL
NPDFIEQVAEFCSYILSHSNVKTLSGGIAVNGPRLESLVLTYVNAIGSGDLPCMENAVLALAQI
ENSAAVEKAIAHYEQQMGQKVQLPTETLQELLDLHRDSEREAIEVFMKNSFKDVDQMFQRKLGA
QLEARRDDFCKQNSKASSDCCMALLQDIFGPLEEDVKQGTFSKPGGYRLFTQKLQELKNKYYQV
PRKGIQAKEVLKKYLESKEDVADALLQTDQSLSEKEKAIEVERIKAESAEAAKKMLEEIQKKNE
EMMEQKEKSYQEHVKQLTEKMERDRAQLMAEQEKTLALKLQEQERLLKEGFENESKRLQKDIWD
IQMRSKSLEPICNIL
GBP3 (GENE No. 106):
NUCLEIC ACID SEQUENCE
(SEQ ID NO: 11)
      1 gatcactgag gaaaatccag aaagctacac aacactgaag gggtgaaata aaagtccagc
    61 gatccagcga aagaaaagag aagtgacaga aacaacttta cctggactga agataaaagc
  121 acagacaaga gaacaatgcc ctggacatgg ctccagagat ccacatgaca ggcccaatgt
  181 gcctcattga gaacactaat ggggaactgg tggcgaatcc agaagctctg aaaatcctgt
  241 ctgccattac acagcctgtg gtggtggtgg caattgtggg cctctaccgc acaggaaaat
  301 cctacctgat gaacaagcta gctgggaaga ataagggctt ctctctgggc tccacagtga
  361 aatctcacac caaaggaatc tggatgtggt gtgtgcctca ccccaaaaag ccagaacaca
  421 ccttagtcct gcttgacact gagggcctgg gagatgtaaa gaagggtgac aaccagaatg
  481 actcctggat cttcaccctg gccgtcctcc tgagcagcac tctcgtgtac aatagcatgg
  541 gaaccatcaa ccagcaggct atggaccaac tgtactatgt gacagagctg acacatcgaa
  601 tccgatcaaa atcctcacct gatgagaatg agaatgagga ttcagctgac tttgtgagct
  661 tcttcccaga ttttgtgtgg acactgagag atttctccct ggacttggaa gcagatggac
  721 aacccctcac accagatgag tacctggagt attccctgaa gctaacgcaa ggtaccagtc
  781 aaaaagataa aaattttaat ctgccccaac tctgtatctg gaagttcttc ccaaagaaaa
  841 aatgttttgt cttcgatctg cccattcacc gcaggaagct tgcccagctt gagaaactac
  901 aagatgaaga gctggaccct gaatttgtgc aacaagtagc agacttctgt tcctacatct
  961 ttagcaattc caaaactaaa actctttcag gaggcatcaa ggtcaatggg cctcgtctag
1021 agagcctagt gctgacctat atcaatgcta tcagcagagg ggatctgccc tgcatggaga
1081 acgcagtcct ggccttggcc cagatagaga actcagccgc agtgcaaaag gctattgccc
1141 actatgacca gcagatgggc cagaaggtgc agctgcccgc agaaaccctc caggagctgc
1201 tggacctgca cagggttagt gagagggagg ccactgaagt ctatatgaag aactctttca
1261 aggatgtgga ccatctgttt caaaagaaat tagcggccca gctagacaaa aagcgggatg
1321 acttttgtaa acagaatcaa gaagcatcat cagatcgttg ctcagcttta ottcaggtca
1381 ttttcagtcc tctagaagaa gaagtgaagg cgggaattta ttcgaaacca gggggctatt
1441 gtctctttat tcagaagcta caagacctgg agaaaaagta ctatgaggaa ccaaggaagg
1501 ggatacaggc tgaagagatt ctgcagacat acttgaaatc caaggagtct gtgaccgatg
1561 caattctaca gacagaccag attctcacag aaaaggaaaa ggagattgaa gtggaatgtg
1621 taaaagctga atctgcacag gcttcagcaa aaatggtgga ggaaatgcaa ataaagtatc
1681 agcagatgat ggaagagaaa gagaagagtt atcaagaaca tgtgaaacaa ttgactgaga
1741 agatggagag ggagagggcc cagttgctgg aagagcaaga gaagaccctc actagtaaac
1801 ttcaggaaca ggcccgagta ctaaaggaga gatgccaagg tgaaagtacc caacttcaaa
1861 atgagataca aaagctacag aagaccctga aaaaaaaaac caagagatat atgtcgcata
1921 agctaaagat ctaaacaaca gagcttttct gtcatcctaa cccaaggcat aactgaaaca
1981 attttagaat ttggaacaag tgtcactata tttgataata attagatctt gcatcataac
2041 actaaaagtt tacaagaaca tgcagttcaa tgatcaaaat catgtttttt ccttaaaaag
2101 attgtaaatt gtgcaacaaa gatgcattta cctctgtacc aacagaggag ggatcatgag
2161 ttgccaccac tcagaagttt attcttccag acgaccagtg gatactgagg aaagtcttag
2221 gtaaaaatct tgggacatat ttgggcactg gtttggccaa gtgtacaatg ggtcccaata
2281 tcagaaacaa ccatcctagc ttcctaggga agacagtgta cagttctcca ttatatcaag
2341 gctacaaggt ctatgagcaa taatgtgatt tctggacatt gcccatggat aattctcact
2401 gatggatctc aagctaaagc aaaccatctt atacagagat ctagaatctt atattttcca
2461 taggaaggta aagaaatcat tagcaagagt aggaattgaa tcataaacaa attggctaat
2521 gaagaaatct tttctttctt gttcaattca tctagattat aaccttaatg tgacacctga
2581 gacctttaga cagttgaccc tgaattaaat agtcacatgg taacaattat gcactgtgta
2641 attttagtaa tgtataacat gcaatgatgc actttaactg aagatagaga ctatgttaga
2701 aaattgaact aatttaatta tttgattgtt ttaatcctaa agcataagtt agtcttttcc
2761 tgattcttaa aggtcatact tgaaatcctg ccaattttcc ccaaagggaa tatggaattt
2821 ttttgacttt cttttgagca ataaaataat tgtcttgcca ttacttagta tatgtagact
2881 tcatcccaat tgtcaaacat cctaggtaag tggttgacat ttcttacagc aattacagat
2941 tatttttgaa ctagaaataa actaaactag aaataaaaaa aaaaaaaaaa aaa
GBP-4 (GENE No. 107):
NUCLEIC ACID SEQUENCE
(SEQ ID NO: 12)
      1 atgggtgaga gaactcttca cgctgcagtg cccacaccag gttatccaga atctgaatcc
    61 atcatgatgg cccccatttg tctagtggaa aaccaggaag agcagctgac agtgaattca
  121 aaggcattag agattcttga caagatttct cagcccgtgg tggtggtggc cattgtaggg
  181 ctataccgca caggaaaatc ctatctcatc aatcgtcttg cagcaaagcg caatggcttc
  241 cctctgggct ccacggtgca gtctgaaact aagggcatct ggatgtggtg tgtgccccac
  301 ctctctaagc caaaccacac cctggtcctt ctggacaccg agggcctggg cgatgtagaa
  361 aagagtaacc ctaagaatga ctcgtggatc tttgccctgg ctgtgcttct aagcagcagc
  421 tttgtctata acagcgtgag caccatcaac caccaggccc tggagcagct gcactatgtg
  481 actgagctag cagagctaat cagggcaaaa tcctgcccca gacctgatga agctgaggac
  541 tccagcgagt ttgcgagttt ctttccagac tttatttgga ctgttcggga ttttaccctg
  601 gagctaaagt tagatggaaa ccccatcaca gaagatgagt acctggagaa tgccttgaag
  661 ctgattccag gcaagaatcc caaaattcaa aattcaaaca tgcctagaga gtgtatcagg
  721 catttcttcc gaaaacggaa gtgctttgtc tttgaccggc ctacaaatga caagcaatat
  781 ttaaatcata tggacgaagt gccagaagaa aatctggaaa ggcatttcct tatgcaatca
  841 gacaacttct gttcttatat cttcacccat gcaaagacca agaccctgag agagggaatc
  901 attgtcactg gaaagcggct ggggactctg gtggtgactt atgtagatgc catcaacagt
  961 ggagcagtac cttgtctgga gaatgcagtg acagcactgg cccagcttga gaacccagcg
1021 gctgtgcaga gggcagccga ccactatagc cagcagatgg cccagcaact gaggctcccc
1081 acagacacgc tccaggagct gctggacgtg catgcagcct gtgagaggga agccattgca
1141 gtcttcatgg agcactcctt caaggatgaa aaccatgaat tccagaagaa gcttgtggac
1201 accatagaga aaaagaaggg agactttgtg ctgcagaatg aagaggcatc tgccaaatat
1261 tgccaggctg agcttaagcg gctttcagag cacctgacag aaagcatttt gagaggaatt
1321 ttctctgttc ctggaggaca caatctctac ttagaagaaa agaaacaggt tgagtgggac
1381 tataagctag tgcccagaaa aggagttaag gcaaacgagg tcctccagaa cttcctgcag
1441 tcacaggtgg ttgtagagga atccatcctg cagtcagaca aagccctcac tgctggagag
1501 aaggccatag cagcggagcg ggccatgaag gaagcagctg agaaggaaca ggagctgcta
1561 agagaaaaac agaaggagca gcagcaaatg atggaggctc aagagagaag cttccaggaa
1621 aacatagctc aactcaagaa gaagatggag agggaaaggg aaaaccttct cagagagcat
1681 gaaaggctgc taaaacacaa gctgaaggta caagaagaaa tgcttaagga agaatttcaa
1741 aagaaatctg agcagttaaa taaagagatt aatcaactga aagaaaaaat tgaaagcact
1801 aaaaatgaac agttaaggct cttaaagatc cttgacatgg ctagcaacat aatgattgtc
1861 actctacctg gggcttccaa gctacttgga gtagggacaa aatatcttgg ctcacgtatt
1921 taa
AMINO ACID SEQUENCE
(SEQ ID NO: 13)
MGERTLHAAVPTPGYPESESIMMAPICLVENQEEQLTVNSKALEILDKISQPVVVVAIVGLYRT
GKSYLMNRLAGKRNGFPLGSTVQSETKGIWMWCVPHLSKPNHTLVLLDTEGLGDVEKSNPKNDS
WIFALAVLLSSSFVYNSVSTINHQALEQLHYVTELAELIRAKSCPRPDEAEDSSEFASFFPDFI
WTVRDFTLELKLDGNPITEDEYLENALKLIPGKNPKIQNSNMPRECIRHFFRKRKCFVFDRPTN
DKQYLNHMDEVPEENLERHFLMQSDNFCSYIFTHAKTKTLREGIIVTGKRLGTLVVTYVDAINS
GAVPCLENAVTALAQLENPAAVQRAADHYSQQMAQQLRLPTDTLQELLDVHAACEREAIAVFME
HSFKDENHEFQKKLVDTIEKKKGDFVLQNEEASAKYCQAELKRLSEHLTESILRGIFSVPGGHN
LYLEEKKQVEWDYKLVPRKGVKANEVLQNFLQSQVVVEESILQSDKALTAGEKAIAAERAMKEA
AEKEQELLREKQKEQQQMMEAQERSFQENIAQLKKKMERERENLLREHERLLKHKLKVQEEMLK
EEFQKKSEQLNKEINQLKEKIESTKNEQLRLLKILDMASNIMIVTLPG
ASKLLGVGTKYLGSRI
GBP-5 (GENE No. 108):
NUCLEIC ACID SEQUENCE
(SEQ ID NO: 14)
      1 ctccaggctg tggaaccttt gttctttcac tctttgcaat aaatcttgct gctgctcact
    61 ctttgggtcc acactgcctt tatgagctgt aacactcact gggaatgtct gcagcttcac
  121 tcctgaagcc agcgagacca cgaacccacc aggaggaaca aacaactcca gacgcgcagc
  181 cttaagagct gtaacactca ccgcgaaggt ctgcagcttc actcctgagc cagccagacc
  241 acgaacccac cagaaggaag aaactccaaa cacatccgaa catcagaagg agcaaactcc
  301 tgacacgcca cctttaagaa ccgtgacact caacgctagg gtccgcggct tcattcttga
  361 agtcagtgag accaagaacc caccaattcc ggacacgcta attgttgtag atcatcactt
  421 caaggtgccc atatctttct agtggaaaaa ttattctggc ctccgctgca tacaaatcag
  481 gcaaccagaa ttctacatat ataaggcaaa gtaacatcct agacatggct ttagagatcc
  541 acatgtcaga ccccatgtgc ctcatcgaga actttaatga gcagctgaag gttaatcagg
  601 aagctttgga gatcctgtct gccattacgc aacctgtagt tgtggtagcg attgtgggcc
  661 tctatcgcac tggcaaatcc tacctgatga acaagctggc tgggaagaac aagggcttct
  721 ctgttgcatc tacggtgcag tctcacacca agggaatttg gatatggtgt gtgcctcatc
  781 ccaactggcc aaatcacaca ttagttctgc ttgacaccga gggcctggga gatgtagaga
  841 aggctgacaa caagaatgat atccagatct ttgcactggc actcttactg agcagcacct
  901 ttgtgtacaa tactgtgaac aaaattgatc agggtgctat cgacctactg cacaatgtga
  961 cagaactgac agatctgctc aaggcaagaa actcacccga ccttgacagg gttgaagatc
1021 ctgctgactc tgcgagcttc ttcccagact tagtgtggac tctgagagat ttctgcttag
1081 gcctggaaat agatgggcaa cttgtcacac cagatgaata cctggagaat tccctaaggc
1141 caaagcaagg tagtgatcaa agagttcaaa atttcaattt gccccgtctg tgtatacaga
1201 agttctttcc aaaaaagaaa tgctttatct ttgacttacc tgctcaccaa aaaaagcttg
1261 cccaacttga aacactgcct gatgatgagc tagagcctga atttgtgcaa caagtgacag
1321 aattctgttc ctacatcttt agccattcta tgaccaagac tcttccaggt ggcatcatgg
1381 tcaatggatc tcgtctaaag aacctggtgc tgacctatgt caatgccatc agcagtgggg
1441 atctgccttg catagagaat gcagtcctgg ccttggctca gagagagaac tcagctgcag
1501 tgcaaaaggc cattgcccac tatgaccagc aaatgggcca gaaagtgcag ctgcccatgg
1561 aaaccctcca ggagctgctg gacctgcaca ggaccagtga gagggaggcc attgaagtct
1621 tcatgaaaaa ctctttcaag gatgtagacc aaagtttcca gaaagaattg gagactctac
1681 tagatgcaaa acagaatgac atttgtaaac ggaacctgga agcatcctcg gattattgct
1741 cggctttact taaggatatt tttggtcctc tagaagaagc agtgaagcag ggaatttatt
1801 ctaagccagg aggccataat ctcttcattc agaaaacaga agaactgaag gcaaagtact
1861 atcgggagcc tcggaaagga atacaggctg aagaagttct gcagaaatat ttaaagtcca
1921 aggagtctgt gagtcatgca atattacaga ctgaccaggc tctcacagag acggaaaaaa
1981 agaagaaaga ggcacaagtg aaagcagaag ctgaaaaggc tgaagcgcaa aggttggcgg
2041 cgattcaaag gcagaacgag caaatgatgc aggagaggga gagactccat caggaacaag
2101 tgagacaaat ggagatagcc aaacaaaatt ggctggcaga gcaacagaaa atgcaggaac
2161 aacagatgca ggaacaggct gcacagctca gcacaacatt ccaagctcaa aatagaagcc
2221 ttctcagtga gctccagcac gcccagagga ctgttaataa cgatgatcca tgtgttttac
2281 tctaaagtgc taaatatggg agtttccttt ttttactctt tgtcactgat gacacaacag
2341 aaaagaaact gtagaccttg ggacaatcaa catttaaata aactttataa ttattttttc
2401 aaactttaaa aaaaaaaaaa aaaaaaaaaa a
AMINO ACID SEQUENCE
(SEQ ID NO: 15)
MALEIHMSDPMCLIENFNEQLKVNQEALEIESAITQPVVVVAIVGLYRTGKSYLMNKLAGKNKG
FSVASTVQSHTKGIWIWCVPHPNWPNHTLVLLDTEGLGDVEKADNKNDIQIFALALLLSSTFVY
NTVNKIDQGAIDLLHNVTELTDLLKARNSPDLDRVEDPADSASFFPDLVWTLRDFCLGLEIDGQ
LVTPDEYLENSLRTKQGSDQRVQNFNTPRLCIQKFFPKKKCFIFDLPAHQKKLAQLETLPDDEL
EPEFVQQVTEFCSYIFSHSMTKTLPGGIMVNGSRLKNLVLTYVNAISSGDLPCIENAVLALAOR
ENSAAVQKAIAHYDQQMGQKVQLPMETLQELLDLHRTSEREAIEVFMKNSFKDVDQSFQKELET
LLDAKQNDICKRNLEASSDYCSALLKDIFGPLEEAVKQGIYSKPGGHNLFIQKTEELKAKYYRE
PRKGIQAEEVLQKYLKSKESVSHAILQTDQALTETEKKKKEAQVKAEAEKAEAQRLAAIQQNEQ
MMQERERLHQEQVRQMEIAKQNWLAEQQKMQEQQMQEQAAQLSTTFQAQNRSLLSELQHAQRTV
NNDDPCVLL
```

### REFERENCES

Baylin, S. B., and J. G. Herman. 2000. DNA hypermethylation in tumorigenesis: epigenetics joins genetics. Trends Genet. 16:168-74.
Bossi, P., G. Viale, A. K. Lee, R. Alfano, G. Coggi, and S. Bosari. 1995. Angiogenesis in colorectal tumors: microvessel quantitation in adenomas and carcinomas with clinicopathological correlations. Cancer Res. 55:5049-53.
Cheng Y S, Colonno R J and Yin F H. Interferon induction of fibroblast proteins with guanylate binding activity. J Biol Chem 1983; 258(12):7746-50.
Choi, H. J., M. S. Hyun, G. J. Jung, S. S. Kim, and S. H. Hong. 1998. Tumor angiogenesis as a prognostic predictor in colorectal carcinoma with special reference to mode of metastasis and recurrence. Oncology. 55:575-81.
Clevers H. At the crossroads of inflammation and cancer. Cell 2004; 118(6):671-4.
Cozzolino, F., M. Torcia, D. Aldinucci, M. Ziche, F. Almerigogna, D. Bani, and D. M. Stern. 1990. Interleukin 1 is an autocrine regulator of human endothelial cell growth. Proc Natl Acad Sci USA. 87:6487-91.
Croner R S, Foertsch T, Brueckl W M, Guenther K, Siebenhaar R, Stremmel C, Matzel K E, Papadopoulos T, Kirchner T, Behrens J, Klein-Hitpass L, Stuerzl M, Hohenberger W and Reingruber B. Common denominator genes that distinguish colorectal carcinoma from normal mucosa. Int J Colorectal Dis 2005a; 20(4):353-62.
Croner R S, Guenther K, Foertsch T, Siebenhaar R, Brueckl W M, Stremmel C, Hlubek F, Hohenberger W and Reingruber B. Tissue preparation for gene expression profiling of colorectal carcinoma: three alternatives to laser microdissection with preamplification. J Lab Clin Med 2004; 143(6):344-51.
Croner R S, Peters A, Brueckl W M, Matzel K E, Klein-Hitpass L, Brabletz T, Papadopoulos T, Hohenberger W, Reingruber B and Lausen B. Microarray versus conventional prediction of lymph node metastasis in colorectal carcinoma. Cancer 2005b; 104(2):395-404.
Ehlert J E, Addison C A, Burdick M D, Kunkel S L and Strieter R M. Identification and partial characterization of a variant of human CXCR3 generated by posttranscriptional exon skipping. J Immunol 2004; 173(10):6234-40.
Fajardo, L. F., H. H. Kwan, J. Kowalski, S. D. Prionas, and A. C. Allison. 1992. Dual role of tumor necrosis factor-alpha in angiogenesis. Am J Pathol. 140:539-44.
Farrell R J and Peppercorn M A. Ulcerative colitis. Lancet 2002; 359(9303):331-40.
Fathallah-Shaykh, H. M., L. J. Zhao, A. I. Kafrouni, G. M. Smith, and J. Forman. 2000. Gene transfer of IFN-gamma into established brain tumors represses growth by antiangiogenesis. J Immunol. 164:217-22.
Frater-Schroder, M., W. Risau, R. Hallmann, P. Gautschi, and P. Bohlen. 1987. Tumor necrosis factor type alpha, a potent inhibitor of endothelial cell growth in vitro, is angiogenic in vivo. Proc Natl Acad Sci USA. 84:5277-81.
Friesel, R., A. Komoriya, and T. Maciag. 1987. Inhibition of endothelial cell proliferation by gamma-interferon. J Cell Biol. 104:689-96.
Gerol, M., L. Curry, L. McCarroll, S. Doctrow, and A. RayChaudhury. 1998. Growth regulation of cultured endothelial cells by inflammatory cytokines: mitogenic, anti-proliferative and cytotoxic effects. Comp Biochem Physiol C Pharmacol Toxicol Endocrinol. 120:397-404.
Guenzi, E., K. Töpolt, E. Cornali, C. Lubeseder-Martellato, A. Jörg, K. Matzen, C. Zietz, E. Kremmer, F. Nappi, M. Schwemmle, C. Hohenadl, G. Barillari, E. Tschachler, P. Monini, B. Ensoli, and M. Stürzl. 2001. The helical domain of GBP-1 mediates the inhibition of endothelial cell proliferation by inflammatory cytokines. EMBO J. 20:5568-77.
Greenwood M. The Natural Duration of Cancer. Rep Publ Hlth Med Subj 1926; 33 (London. H M Stationary Office):
Guenzi, E., K. Töpolt, C. Lubeseder-Martellato, A. Jörg, E. Naschberger, R. Benelli, A. Albini, and M. Stürzl. 2003. The guanylate binding protein-1 GTPase controls the invasive and angiogenic capability of endothelial cells through inhibition of MMP-1 expression. EMBO J. 22:3772-82.
Guenzi E, Töpolt K, Cornali E, Lubeseder-Martellato C, Jörg A, Matzen K, Zietz C, Kremmer E, Nappi F, Schwemmle M, Hohenadl C, Barillari G, Tschachler E, Monini P, Ensoli B and Stürzl M. The helical domain of GBP-1 mediates the inhibition of endothelial cell proliferation by inflammatory cytokines. EMBO J 2001; 20(20):5568-77.
Guenzi E, Töpolt K, Lubeseder-Martellato C. Jörg A, Naschberger E, Benelli R, Albini A and Stürzl M. The guanylate binding protein-1 GTPase controls the invasive and angiogenic capability of endothelial cells through inhibition of MMP-1 expression. EMBO J 2003; 22(15):3772-82.
Hawk, E. T., and B. Levin. 2005. Colorectal cancer prevention. J Clin Oncol. 23:378-91.
Hurwitz, H., L. Fehrenbacher, W. Novotny, T. Cartwright, J. Hainsworth, W. Heim, J. Berlin, A. Baron, S. Griffing, E. Holmgren, N. Ferrara, G. Fyfe, B. Rogers, R. Ross, and F. Kabbinavar. 2004. Bevacizumab plus irinotecan, fluorouracil, and leucovorin for metastatic colorectal cancer. N Engl J Med. 350:2335-42.
Ilyas, M., J. Straub, I. P. Tomlinson, and W. F. Bodmer. 1999. Genetic pathways in colorectal and other cancers. Eur J Cancer. 35:1986-2002.
Ishigami, S. I., S. Arii, M. Furutani, M. Niwano, T. Harada, M. Mizumoto, A. Mori, H. Onodera, and M. Imamura. 1998. Predictive value of vascular endothelial growth factor (VEGF) in metastasis and prognosis of human colorectal cancer. Br J Cancer. 78:1379-84.
Itzkowitz S H and Yio X. Inflammation and cancer IV. Colorectal cancer in inflammatory bowel disease: the role of inflammation. Am J Physiol Gastrointest Liver Physiol 2004; 287(1):G7-17.
Jass J R and Sobin L H (1989). Histological Classification of Tumours. Berlin Heidelberg New York, Springer.
Jass, J. R. 2002. Pathogenesis of colorectal cancer. Surg Clin North Am. 82:891-904.
Joseph. I. B., and J. T. Isaacs. 1998. Macrophage role in the anti-prostate cancer response to one class of antiangiogenic agents. J Natl Cancer Inst. 90:1648-53.
Kahlenberg, M. S., J. M. Sullivan, D. D. Witmer, and N. J. Petrelli. 2003. Molecular prognostics in colorectal cancer. Surg Oncol. 12:173-86.
Kang, S. M., K. Maeda, N. Onoda, Y. S. Chung. B. Nakata, Y. Nishiguchi, and M. Sowa. 1997. Combined analysis of p53 and vascular endothelial growth factor expression in colorectal carcinoma for determination of tumor vascularity and liver metastasis. Int J Cancer. 74:502-7.
Kumar, H., K. Heer, P. W. Lee, G. S. Duthie, A. W. MacDonald, J. Greenman, M. J. Kerin, and J. R. Monson. 1998. Preoperative serum vascular endothelial growth factor can predict stage in colorectal cancer. Clin Cancer Res. 4:1279-85.
Lasagni L, Francalanci M, Annunziato F, Lazzeri E, Giannini S, Cosmi L, Sagrinati C, Mazzinghi B, Orlando C, Maggi E, Marra F, Romagnani S, Serio M and Romagnani P. An alternatively spliced variant of CXCR3 mediates the inhibition of endothelial cell growth induced by IP-10, Mig, and I-TAC, and acts as functional receptor for platelet factor 4. J Exp Med 2003; 197(11):1537-49.
Lubeseder-Martellato C, Guenzi E, Jörg A. Töpolt K, Naschberger E, Kremmer E, Zietz C, Tschachler E, Hutzler P, Schwemmle M, Matzen K, Grimm T, Ensoli B and Stürzl M. Guanylate-Binding Protein-1 Expression Is Selectively Induced by Inflammatory Cytokines and Is an Activation Marker of Endothelial Cells during Inflammatory Diseases. Am J Pathol 2002; 161(5): 1749-59.
Mahadevan, V., I. R. Hart, and G. P. Lewis. 1989. Factors influencing blood supply in wound granuloma quantitated by a new in vivo technique. Cancer Res. 49:415-9.
Montrucchio, G., E. Lupia, E. Battaglia, G. Passerini, F. Bussolino, G. Emanuelli, and G. Camussi. 1994. Tumor necrosis factor alpha-induced angiogenesis depends on in situ platelet-activating factor biosynthesis. J Exp Med. 180:377-82.
Naschberger E, Bauer M and Stürzl M. Human guanylate binding protein-1 (hGBP-1) characterizes and establishes a non-angiogenic endothelial cell activation phenotype in inflammatory diseases. Adv Enzyme Regul 2005; 45(215-27.
Naschberger E, Werner T, Vicente A B, Guenzi E, Töpolt K, Leubert R, Lubeseder-Martellato C, Nelson P J and Stürzl M. Nuclear factor-kappaB motif and interferon-alpha-stimulated response element co-operate in the activation of guanylate-binding protein-1 expression by inflammatory cytokines in endothelial cells. Biochem J 2004; 379(Pt 2):409-20.
Naschberger, E., M. Bauer, and M. Sturzl. 2005. Human guanylate binding protein-1 (hGBP-1) characterizes and establishes a non-angiogenic endothelial cell activation phenotype in inflammatory diseases. Adv Enzyme Regul. 45:215-27.
Negrier S, Escudier B, Lasset C, Douillard J Y, Savary J, Chevreau C, Ravaud A, Mercatello A, Peny J, Mousseau M, Philip T and Tursz T. Recombinant human interleukin-2, recombinant human interferon alfa-2a, or both in metastatic renal-cell carcinoma. Groupe Francais d'Immunotherapie. N Engl J Med 1998; 338(18):1272-8.
Norioka, K., T. Mitaka, Y. Mochizuki, M. Hara, M. Kawagoe, and H. Nakamura. 1994. Interaction of interleukin-1 and interferon-gamma on fibroblast growth factor-induced angiogenesis. Jpn J Cancer Res. 85:522-9.
Prakash B, Praefcke G J, Renault L, Wittinghofer A and Herrmann C. Structure of human guanylate-binding protein 1 representing a unique class of GTP-binding proteins. Nature 2000; 403(6769):567-71.
Romagnani P. Annunziato F, Lasagni L, Lazzeri E, Beltrame C, Francalanci M, Uguccioni M, Galli G, Cosmi L, Maurenzig L, Baggiolini M, Maggi E, Romagnani S and Serio M. Cell cycle-dependent expression of CXC chemokine receptor 3 by endothelial cells mediates angiostatic activity. J Clin Invest 2001; 107(1):53-63.
Romagnani P, Lasagni L, Annunziato F, Serio M and Romagnani S. CXC chemokines: the regulatory link between inflammation and angiogenesis. Trends Immunol 2004; 25(4):201-9.
Samaniego, F., P. D. Markham, R. Gendelman, R. C. Gallo, and B. Ensoli. 1997. Inflammatory cytokines induce endothelial cells to produce and release basic fibroblast growth factor and to promote Kaposi's sarcoma-like lesions in nude mice. J Immunol. 158:1887-94.
Schweigerer, L., B. Malerstein, and D. Gospodarowicz. 1987. Tumor necrosis factor inhibits the proliferation of cultured capillary endothelial cells. Biochem Biophys Res Commun. 143:997-1004.
Smith, R. A., V. Cokkinides, A. C. von Eschenbach, B. Levin, C. Cohen, C. D. Runowicz, S. Sener, D. Saslow, and H. J. Eyre. 2002. American Cancer Society guidelines for the early detection of cancer. CA Cancer J Clin. 52:8-22.
Soreide O, Norstein J, Fielding L P and Silen W (1997). International standardization and documentation of the treatment of rectal cancer. Berlin Heidelberg New York, Springer.
Spinetti G, Camarda G, Bernardini G, Romano Di Peppe S, Capogrossi M C and Napolitano M. The chemokine CXCL13 (BCA-1) inhibits FGF-2 effects on endothelial cells. Biochem Biophys Res Commun 2001; 289(1):19-24.
Strieter R M, Belperio J A, Burdick M D and Keane M P. CXC chemokines in angiogenesis relevant to chronic fibroproliferation. Curr Drug Targets Inflamm Allergy 2005a; 4(1):23-6.
Strieter R M, Belperio J A, Phillips R J and Keane M P. CXC chemokines in angiogenesis of cancer. Semin Cancer Biol 2004; 14(3): 195-200.
Strieter R M, Burdick M D, Gomperts B N, Belperio J A and Keane M P. CXC chemokines in angiogenesis. Cytokine Growth Factor Rev 2005b; 16(6):593-609.
Strieter R M, Burdick M D, Mestas J, Gomperts B, Keane M P and Belperio J A. Cancer CXC chemokine networks and tumour angiogenesis. Eur J Cancer 2006; 42(6):768-778.
Stürzl M, Hohenadl C, Zietz C, Castanos-Velez E, Wunderlich A, Ascherl G, Biberfeld P, Monini P, Browning P J and Ensoli B. Expression of K13/v-FLIP gene of human herpesvirus 8 and apoptosis in Kaposi's sarcoma spindle cells. J Natl Cancer Inst 1999; 91(20):1725-33.
Stürzl M, Roth W K, Brockmeyer N H, Zietz C, Speiser B and Hofschneider P H. Expression of platelet-derived growth factor and its receptor in AIDS-related Kaposi sarcoma in vivo suggests paracrine and autocrine mechanisms of tumor maintenance. Proc Natl Acad Sci USA 1992; 89(15):7046-50.
Takayama, T., M. Ohi, T. Hayashi, K. Miyanishi, A. Nobuoka, T. Nakajima, T. Satoh, R. Takimoto, J. Kato, S. Sakamaki, and Y. Niitsu. 2001. Analysis of K-ras, APC, and beta-catenin in aberrant crypt foci in sporadic adenoma, cancer, and familial adenomatous polyposis. Gastroenterology. 121:599-611.
Takebayashi, Y., S. Aklyama, K. Yamada, S. Akiba, and T. Aikou. 1996. Angiogenesis as an unfavorable prognostic factor in human colorectal carcinoma. Cancer. 78:226-31.
Torisu, H., M. Ono, H. Kiryu, M. Furue, Y. Ohmoto, J. Nakayama, Y. Nishioka, S. Sone, and M. Kuwano. 2000. Macrophage infiltration correlates with tumor stage and angiogenesis in human malignant melanoma: possible involvement of TNFalpha and IL-1alpha. Int J Cancer. 85:182-8.
Vogelstein, B., E. R. Fearon, S. R. Hamilton, S. E. Kern, A. C. Preisinger, M. Leppert, Y. Nakamura, R. White, A. M. Smits, and J. L. Bos. 1988. Genetic alterations during colorectal-tumor development. N Engl J Med. 319:525-32.
Yilmaz, A., G. Bieler, O. Spertini, F. J. Lejeune, and C. Ruegg. 1998. Pulse treatment of human vascular endothelial cells with high doses of tumor necrosis factor and interferon-gamma results in simultaneous synergistic and reversible effects on proliferation and morphology. Int J Cancer. 77:592-9.

What is claimed is:

1. An ex vivo method for the detection of an angiostatic tumor stage/tumor area of colorectal carcinoma in a patient comprising a detection step using a microarray, wherein the microarray comprises gene probes capable of specifically hybridizing to the nucleic acids according to GENE Nos. 1-108 or derivatives thereof, wherein the array comprises gene probes hybridizing to a subset of at least 4 of the above nucleic acid sequences, and further wherein the array comprises gene probes specifically hybridizing to the nucleic acid sequences of GENE Nos. 1, 4, 8 and 41.

2. The method of claim 1, wherein the array further comprises gene probes capable of specifically hybridizing to at least one of the nucleic acids according to GENE Nos. 109-157.

3. The method of claim 1, wherein the array further comprises appropriate control gene probes, optionally wherein the control gene is actin or GAPDH.

4. The method of claim 1, wherein the array further comprises gene probes capable of hybridizing to the nucleic acid sequences of GENE Nos. 1, 4, 8, 14, 25, 26, 41, 59, 65, 76, 81, 105, 106, 107, and 108.

5. The method of claim 1, wherein the gene probes are oligonucleotides, cDNA, RNA, or PNA molecules.

6. The method of claim 1, wherein the nucleic acids further comprise a label selected from the group consisting of a radioactive label, a fluorescent label, biotin, digoxigenin, a peroxidase label, a label detectable by alkaline phosphatase, or a combination thereof.

7. The method of claim 1, wherein the gene probes of the array are bound to a solid phase matrix, optionally wherein the solid phase matrix comprises a nylon membrane, glass, or a plastic.

8. An ex vivo method for the diagnosis of an angiostatic tumor stage/tumor area in a CRC patient, the method comprising:
(a) providing a sample of the patient;
(b) extracting RNA from the sample;
(c) optionally transcribing RNA to cDNA or cRNA; and
(d) detecting whether at least four nucleic acid sequences selected from the group consisting of GENE Nos. 1-108 are present in the sample, and whether the sample contains at least the nucleic acid sequences of GENE Nos. 1, 4, 8 and 41,
wherein the presence of said nucleic acids is indicative for the presence of an angiostatic tumor stage/tumor area of CRC in said patient.

9. The method of claim 8, wherein the sample is a CRC tissue sample or a cell lysate or a body fluid sample.

10. The method of claim 9, wherein the detection is performed by RT-PCR.

11. The method of claim 10, wherein the RT-PCR is multiplex RT-PCR.

12. The method of claim 8, wherein the detection is performed by means of complementary gene probes.

13. The method of claim 12, wherein the gene probes are cDNA or oligonucleotide probes.

14. The method of claim 13, wherein the detection is performed using gene probes that are capable of hybridizing to at least a portion of the nucleic acid sequences of GENE Nos. 1-108, or to RNA sequences or derivatives derived therefrom.

15. The method of claim 14, wherein a microarray as defined in claim 1 is used for the detection.

16. The method of claim 14, wherein the hybridization is performed under moderately stringent conditions.

17. An ex vivo method for the diagnosis of an angiostatic tumor stage/tumor area in a CRC, the method comprising:
(a) providing a sample from the patient; and
(b) detecting whether at least four amino acid sequences corresponding to the nucleic acid sequences selected from the group consisting of GENE Nos. 1-108 are present in the sample, and whether the sample contains at least the amino acids corresponding to the nucleic acid sequences of Seq. No. 1, 4, 8 and 41;
wherein the presence of said proteins is indicative for the presence of an angiostatic tumor stage/tumor area of CRC in said patient.

18. The method of claim 17, wherein the detection is performed by contacting the sample with antibodies that specifically recognize an amino acid sequence encoded by a nucleic acid sequence of one of GENE Nos. 1-108.

19. The method of claim 17, wherein the sample is a CRC tissue sample, a cell lysate, or a body fluid.

20. The method of claim 17, wherein the amino acid sequences are detected using multiplex Western blot or ELISA.


SEQUENCE LISTING (60 sequences)

SEQ ID NO: 1 - DNA, 2545 bp, Homo sapiens
  CDS (40)..(417)
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 2 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 3 - DNA, 1172 bp, Homo sapiens
  CDS (67)..(363)
gagacattcctcaattgcttagacatattctgagcctacagcagaggaacctccagtctcagcaccatgaatcaaactgcgattctgatttgctgccttatctttctgactctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatattaagaaagactatctaaatgttgaaagatcaaaaggttaataaagtaattataact

SEQ ID NO: 4 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKEMSKRSP

SEQ ID NO: 5 - DNA, 1493 bp, Homo sapiens
  CDS (94)..(378)
ttcctttcatgttcagcatttctactccttccaagaagagcagcaaagctgaagtagcagcaacagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagggcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccagcaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtcaaaagcatctatgtgtcgtaaagcattcctcaaacattttttcatgcaaatacacacttctttccccaaatatcatgtagcacatcaatatgtagggaaacattcttatgcatcatttggtttgttttataaccaattcattaaatgtaattcataaaatgtactatgaaaaaaattatacgctatgggatactggcaacagtgcacatatttcataaccaaattagcagcaccggtcttaatttgatgtttttcaacttttattcattgagatgttttgaagcaattaggatatgtgtgtttactgtactttttgttttgatccgtttgtataaatgatagcaatatcttggacacatttgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaaaaaa

SEQ ID NO: 6 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 7 - DNA, 1803 bp, Homo sapiens
  CDS (5)..(1783)
ggacatggcatcagagatccacatgacaggcccaatgtgcctcattgagaacactaatgggcgactgatggcgaatccagaagctctgaagatcctttctgccattacacagcctatggtggtggtggcaattgtgggcctctaccgcacaggcaaatcctacctgatgaacaagctggctggaaagaaaaagggcttctctctgggctccacggtgcagtctcacactaaaggaatctggatgtggtgtgtgccccaccccaagaagccaggccacatcctagttctgctggacaccgagggtctgggagatgtagagaagggtgacaaccagaatgactcctggatcttcgccctggccgtcctcctgagcagcaccttcgtgtacaatagcataggaaccatcaaccagcaggctatggaccaactgtactatgtgacagagctgacacatagaatccgatcaaaatcctcacctgatgagaatgagaatgaggttgaggattcagctgactttgtgagcttcttcccagactttgtgtggacactgagagatttctccctggacttggaagcagatggacaacccctcacaccagatgagtacctgacatactccctgaagctgaagaaaggtaccagtcaaaaagatgaaacttttaacctgcccagactctgtatccggaaattcttcccaaagaaaaaatgctttgtctttgatcggcccgttcaccgcaggaagcttgcccagctcgagaaactacaagatgaagagctggaccccgaatttgtgcaacaagtagcagacttctgttcctacatctttagtaattccaaaactaaaactctttcaggaggcatccaggtcaacgggcctcgtctagagagcctggtgctgacctacgtcaatgccatcagcagtggggatctgccgtgcatggagaacgcagtcctggccttggcccagatagagaactcagctgcagtgcaaaaggctattgcccactatgaacagcagatgggccagaaggtgcagctgcccacagaaagcctccaggagctgctggacctgcacagggacagtgagagagaggccattgaagtcttcatcaggagttccttcaaagatgtggaccatctatttcaaaaggagttagcggcccagctagaaaaaaagcgggatgacttttgtaaacagaatcaggaagcatcatcagatcgttgctcagctttacttcaggtcattttcagtcctctagaagaagaagtgaaggcgggaatttattcgaaaccagggggctatcgtctctttgttcagaagctacaagacctgaagaaaaagtactatgaggaaccgaggaaggggatacaggctgaagagattctgcagacatacttgaaatccaaggagtctatgactgatgcaattctccagacagaccagactctcacagaaaaagaaaaggagattgaagtggaacgtgtgaaagctgagtctgcacaggcttcagcaaaaatgttgcaggaaatgcaaagaaagaatgagcagatgatggaacagaaggagaggagttatcaggaacacttgaaacaactgactgagaagatggagaacgacagggtccagttgctgaaagagcaagagaggaccctcgctcttaaacttcaggaacaggagcaactactaaaagagggatttcaaaaagaaagcagaataatgaaaaatgagatacaggatctccagacgaaaatgagacgacgaaaggcatgtaccataagctaaagaccagagccttcctgtca

SEQ ID NO: 8 - protein, 592 aa, Homo sapiens [sequence omitted]

SEQ ID NO: 9 - DNA, 1776 bp, Homo sapiens
  CDS (1)..(1776)
atggctccagagatcaacttgccgggcccaatgagcctcattgataacactaaagggcagctggtggtgaatccagaagctctgaagatcctatctgcaattacgcagcctgtggtggtggtggcgattgtgggcctctatcgcacaggcaaatcctacctgatgaacaagctggctgggaagaaaaacggcttctctctaggctccacagtgaagtctcacaccaagggaatctggatgtggtgtgtgcctcatcccaagaagccagaacacaccctagttctgctcgacactgagggcctgggagatatagagaagggtgacaatgagaatgactcctggatctttgccttggccatcctcctgagcagcaccttcgtgtacaatagcatgggaaccatcaaccagcaggccatggaccaacttcactatgtgacagagctgacagatcgaatcaaggcaaactcctcacctggtaacaattctgtagacgactcagctgactttgtgagcttttttccagcatttgtgtggactctcagagatttcaccctggaactggaagtagatggagaacccatcactgctgatgactacttggagctttcgctaaagctaagaaaaggtactgataagaaaagtaaaagctttaatgatcctcggttgtgcatccgaaagttcttccccaagaggaagtgcttcgtcttcgattggcccgctcctaagaagtaccttgctcacctagagcagctaaaggaggaagagctgaaccctgatttcatagaacaagttgcagaattttgttcctacatcctcagccattccaatgtcaagactctttcaggtggcattgcagtcaatgggcctcgtctagagagcctggtgctgacctacgtcaatgccatcggcagtggggatctaccctgcatggagaacgcagtcctggccttggcccagatagagaactcagccgcagtggaaaaggctattgcccactatgaacagcagatgggccagaaggtgcagctgcccacggaaaccctccaggagctgctggacctgcacagggacagtgagagagaggccattgaagtcttcatgaagaactctttcaaggatgtggaccaaatgttccagaggaaattaggggcccagttggaagcaaggcgagatgacttttgtaagcagaattccaaagcatcatcagattgttgcatggctttacttcaggatatatttggccctttagaagaagatgtcaagcagggaacattttctaaaccaggaggttaccgtctctttactcagaagctgcaggagctgaagaataagtactaccaggtgccaaggaaggggatacaggccaaagaggtgctgaaaaaatatttggagtccaaggaggatgtggctgatgcacttctacagactgatcagtcactctcagaaaaggaaaaagcgattgaagtggaacgtataaaggctgaatctgcagaagctgcaaagaaaatgttggaggaaatacaaaagaagaatgaggagatgatggaacagaaagagaagagttatcaggaacatgtgaaacaattgactgagaagatggagagggacagggcccagttaatggcagagcaagagaagaccctcgctcttaaacttcaggaacaggaacgccttctcaaggagggattcgagaatgagagcaagagacttcaaaaagacatatgggatatccagatgagaagcaaatcattggagccaatatgtaacatactttaa

SEQ ID NO: 10 - protein, 591 aa, Homo sapiens [sequence omitted]

SEQ ID NO: 11 - DNA, 2993 bp, Homo sapiens
gatcactgaggaaaatccagaaagctacacaacactgaaggggtgaaataaaagtccagcgatccagcgaaagaaaagagaagtgacagaaacaactttacctggactgaagataaaagcacagacaagagaacaatgccctggacatggctccagagatccacatgacaggcccaatgtgcctcattgagaacactaatggggaactggtggcgaatccagaagctctgaaaatcctgtctgccattacacagcctgtggtggtggtggcaattgtgggcctctaccgcacaggaaaatcctacctgatgaacaagctagctgggaagaataagggcttctctctgggctccacagtgaaatctcacaccaaaggaatctggatgtggtgtgtgcctcaccccaaaaagccagaacacaccttagtcctgcttgacactgagggcctgggagatgtaaagaagggtgacaaccagaatgactcctggatcttcaccctggccgtcctcctgagcagcactctcgtgtacaatagcatgggaaccatcaaccagcaggctatggaccaactgtactatgtgacagagctgacacatcgaatccgatcaaaatcctcacctgatgagaatgagaatgaggattcagctgactttgtgagcttcttcccagattttgtgtggacactgagagatttctccctggacttggaagcagatggacaacccctcacaccagatgagtacctggagtattccctgaagctaacgcaaggtaccagtcaaaaagataaaaattttaatctgccccaactctgtatctggaagttcttcccaaagaaaaaatgttttgtcttcgatctgcccattcaccgcaggaagcttgcccagcttgagaaactacaagatgaagagctggaccctgaatttgtgcaacaagtagcagacttctgttcctacatctttagcaattccaaaactaaaactctttcaggaggcatcaaggtcaatgggcctcgtctagagagcctagtgctgacctatatcaatgctatcagcagaggggatctgccctgcatggagaacgcagtcctggccttggcccagatagagaactcagccgcagtgcaaaaggctattgcccactatgaccagcagatgggccagaaggtgcagctgcccgcagaaaccctccaggagctgctggacctgcacagggttagtgagagggaggccactgaagtctatatgaagaactctttcaaggatgtggaccatctgtttcaaaagaaattagcggcccagctagacaaaaagcgggatgacttttgtaaacagaatcaagaagcatcatcagatcgttgctcagctttacttcaggtcattttcagtcctctagaagaagaagtgaaggcgggaatttattcgaaaccagggggctattgtctctttattcagaagctacaagacctggagaaaaagtactatgaggaaccaaggaaggggatacaggctgaagagattctgcagacatacttgaaatccaaggagtctgtgaccgatgcaattctacagacagaccagattctcacagaaaaggaaaaggagattgaagtggaatgtgtaaaagctgaatctgcacaggcttcagcaaaaatggtggaggaaatgcaaataaagtatcagcagatgatggaagagaaagagaagagttatcaagaacatgtgaaacaattgactgagaagatggagagggagagggcccagttgctggaagagcaagagaagaccctcactagtaaacttcaggaacaggcccgagtactaaaggagagatgccaaggtgaaagtacccaacttcaaaatgagatacaaaagctacagaagaccctgaaaaaaaaaaccaagagatatatgtcgcataagctaaagatctaaacaacagagcttttctgtcatcctaacccaaggcataactgaaacaattttagaatttggaacaagtgtcactatatttgataataattagatcttgcatcataacactaaaagtttacaagaacatgcagttcaatgatcaaaatcatgttttttccttaaaaagattgtaaattgtgcaacaaagatgcatttacctctgtaccaacagaggagggatcatgagttgccaccactcagaagtttattcttccagacgaccagtggatactgaggaaagtcttaggtaaaaatcttgggacatatttgggcactggtttggccaagtgtacaatgggtcccaatatcagaaacaaccatcctagcttcctagggaagacagtgtacagttctccattatatcaaggctacaaggtctatgagcaataatgtgatttctggacattgcccatggataattctcactgatggatctcaagctaaagcaaaccatcttatacagagatctagaatcttatattttccataggaaggtaaagaaatcattagcaagagtaggaattgaatcataaacaaattggctaatgaagaaatcttttctttcttgttcaattcatctagattataaccttaatgtgacacctgagacctttagacagttgaccctgaattaaatagtcacatggtaacaattatgcactgtgtaattttagtaatgtataacatgcaatgatgcactttaactgaagatagagactatgttagaaaattgaactaatttaattatttgattgttttaatcctaaagcataagttagtcttttcctgattcttaaaggtcatacttgaaatcctgccaattttccccaaagggaatatggaatttttttgactttcttttgagcaataaaataattgtcttgccattacttagtatatgtagacttcatcccaattgtcaaacatcctaggtaagtggttgacatttcttacagcaattacagattatttttgaactagaaataaactaaactagaaataaaaaaaaaaaaaaaaaaa

SEQ ID NO: 12 - DNA, 1923 bp, Homo sapiens
  CDS (1)..(1923)
atgggtgagagaactcttcacgctgcagtgcccacaccaggttatccagaatctgaatccatcatgatggcccccatttgtctagtggaaaaccaggaagagcagctgacagtgaattcaaaggcattagagattcttgacaagatttctcagcccgtggtggtggtggccattgtagggctataccgcacaggaaaatcctatctcatgaatcgtcttgcaggaaagcgcaatggcttccctctgggctccacggtgcagtctgaaactaagggcatctggatgtggtgtgtgccccacctctctaagccaaaccacaccctggtccttctggacaccgagggcctgggcgatgtagaaaagagtaaccctaagaatgactcgtggatctttgccctggctgtgcttctaagcagcagctttgtctataacagcgtgagcaccatcaaccaccaggccctggagcagctgcactatgtgactgagctagcagagctaatcagggcaaaatcctgccccagacctgatgaagctgaggactccagcgagtttgcgagtttctttccagactttatttggactgttcgggattttaccctggagctaaagttagatggaaaccccatcacagaagatgagtacctggagaatgccttgaagctgattccaggcaagaatcccaaaattcaaaattcaaacatgcctagagagtgtatcaggcatttcttccgaaaacggaagtgctttgtctttgaccggcctacaaatgacaagcaatatttaaatcatatggacgaagtgccagaagaaaatctggaaaggcatttccttatgcaatcagacaacttctgttcttatatcttcacccatgcaaagaccaagaccctgagagagggaatcattgtcactggaaagcggctggggactctggtggtgacttatgtagatgccatcaacagtggagcagtaccttgtctggagaatgcagtgacagcactggcccagcttgagaacccagcggctgtgcagagggcagccgaccactatagccagcagatggcccagcaactgaggctccccacagacacgctccaggagctgctggacgtgcatgcagcctgtgagagggaagccattgcagtcttcatggagcactccttcaaggatgaaaaccatgaattccagaagaagcttgtggacaccatagagaaaaagaagggagactttgtgctgcagaatgaagaggcatctgccaaatattgccaggctgagcttaagcggctttcagagcacctgacagaaagcattttgagaggaattttctctgttcctggaggacacaatctctacttagaagaaaagaaacaggttgagtgggactataagctagtgcccagaaaaggagttaaggcaaacgaggtcctccagaacttcctgcagtcacaggtggttgtagaggaatccatcctgcagtcagacaaagccctcactgctggagagaaggccatagcagcggagcgggccatgaaggaagcagctgagaaggaacaggagctgctaagagaaaaacagaaggagcagcagcaaatgatggaggctcaagagagaagcttccaggaaaacatagctcaactcaagaagaagatggagagggaaagggaaaaccttctcagagagcatgaaaggctgctaaaacacaagctgaaggtacaagaagaaatgcttaaggaagaatttcaaaagaaatctgagcagttaaataaagagattaatcaactgaaagaaaaaattgaaagcactaaaaatgaacagttaaggctcttaaagatccttgacatggctagcaacataatgattgtcactctacctggggcttccaagctacttggagtagggacaaaatatcttggctcacgtatttaa

SEQ ID NO: 13 - protein, 640 aa, Homo sapiens
MGERTLHAAVPTPGYPESESIMMAPICLVENQEEQLTVNSKALEILDKISQPVVVVAIVGLYRTGKSYLMNRLAGKRNGFPLGSTVQSETKGIWMWCVPHLSKPNHTLVLLDTEGLGDVEKSNPKNDSWIFALAVLLSSSFVYNSVSTINHQALEQLHYVTELAELIRAKSCPRPDEAEDSSEFASFFPDFIWTVRDFTLELKLDGNPITEDEYLENALKLIPGKNPKIQNSNMPRECIRHFFRKRKCFVFDRPTNDKQYLNHMDEVPEENLERHFLMQSDNFCSYIFTHAKTKTLREGIIVTGKRLGTLVVTYVDAINSGAVPCLENAVTALAQLENPAAVQRAADHYSQQMAQQLRLPTDTLQELLDVHAACEREAIAVFMEHSFKDENHEFQKKLVDTIEKKKGDFVLQNEEASAKYCQAELKRLSEHLTESILRGIFSVPGGHNLYLEEKKQVEWDYKLVPRKGVKANEVLQNFLQSQVVVEESILQSDKALTAGEKAIAAERAMKEAAEKEQELLREKQKEQQQMMEAQERSFQENIAQLKKKMERERENLLREHERLLKHKLKVQEEMLKEEFQKKSEQLNKEINQLKEKIESTKNEQLRLLKILDMASNIMIVTLPGASKLLGVGTKYLGSRI

SEQ ID NO: 14 - DNA, 2431 bp, Homo sapiens
  CDS (525)..(2285)
ctccaggctgtggaacctttgttctttcactctttgcaataaatcttgctgctgctcactctttgggtccacactgcctttatgagctgtaacactcactgggaatgtctgcagcttcactcctgaagccagcgagaccacgaacccaccaggaggaacaaacaactccagacgcgcagccttaagagctgtaacactcaccgcgaaggtctgcagcttcactcctgagccagccagaccacgaacccaccagaaggaagaaactccaaacacatccgaacatcagaaggagcaaactcctgacacgccacctttaagaaccgtgacactcaacgctagggtccgcggcttcattcttgaagtcagtgagaccaagaacccaccaattccggacacgctaattgttgtagatcatcacttcaaggtgcccatatctttctagtggaaaaattattctggcctccgctgcatacaaatcaggcaaccagaattctacatatataaggcaaagtaacatcctagacatggctttagagatccacatgtcagaccccatgtgcctcatcgagaactttaatgagcagctgaaggttaatcaggaagctttggagatcctgtctgccattacgcaacctgtagttgtggtagcgattgtgggcctctatcgcactggcaaatcctacctgatgaacaagctggctgggaagaacaagggcttctctgttgcatctacggtgcagtctcacaccaagggaatttggatatggtgtgtgcctcatcccaactggccaaatcacacattagttctgcttgacaccgagggcctgggagatgtagagaaggctgacaacaagaatgatatccagatctttgcactggcactcttactgagcagcacctttgtgtacaatactgtgaacaaaattgatcagggtgctatcgacctactgcacaatgtgacagaactgacagatctgctcaaggcaagaaactcacccgaccttgacagggttgaagatcctgctgactctgcgagcttcttcccagacttagtgtggactctgagagatttctgcttaggcctggaaatagatgggcaacttgtcacaccagatgaatacctggagaattccctaaggccaaagcaaggtagtgatcaaagagttcaaaatttcaatttgccccgtctgtgtatacagaagttctttccaaaaaagaaatgctttatctttgacttacctgctcaccaaaaaaagcttgcccaacttgaaacactgcctgatgatgagctagagcctgaatttgtgcaacaagtgacagaattctgttcctacatctttagccattctatgaccaagactcttccaggtggcatcatggtcaatggatctcgtctaaagaacctggtgctgacctatgtcaatgccatcagcagtggggatctgccttgcatagagaatgcagtcctggccttggctcagagagagaactcagctgcagtgcaaaaggccattgcccactatgaccagcaaatgggccagaaagtgcagctgcccatggaaaccctccaggagctgctggacctgcacaggaccagtgagagggaggccattgaagtcttcatgaaaaactctttcaaggatgtagaccaaagtttccagaaagaattggagactctactagatgcaaaacagaatgacatttgtaaacggaacctggaagcatcctcggattattgctcggctttacttaaggatatttttggtcctctagaagaagcagtgaagcagggaatttattctaagccaggaggccataatctcttcattcagaaaacagaagaactgaaggcaaagtactatcgggagcctcggaaaggaatacaggctgaagaagttctgcagaaatatttaaagtccaaggagtctgtgagtcatgcaatattacagactgaccaggctctcacagagacggaaaaaaagaagaaagaggcacaagtgaaagcagaagctgaaaaggctgaagcgcaaaggttggcggcgattcaaaggcagaacgagcaaatgatgcaggagagggagagactccatcaggaacaagtgagacaaatggagatagccaaacaaaattggctggcagagcaacagaaaatgcaggaacaacagatgcaggaacaggctgcacagctcagcacaacattccaagctcaaaatagaagccttctcagtgagctccagcacgcccagaggactgttaataacgatgatccatgtgttttactctaaagtgctaaatatgggagtttcctttttttactctttgtcactgatgacacaacagaaaagaaactgtagaccttgggacaatcaacatttaaataaactttataattattttttcaaactttaaaaaaaaaaaaaaaaaaaaaaaa

SEQ ID NO: 15 - protein, 586 aa, Homo sapiens
MALEIHMSDPMCLIENFNEQLKVNQEALEILSAITQPVVVVAIVGLYRTGKSYLMNKLAGKNKGFSVASTVQSHTKGIWIWCVPHPNWPNHTLVLLDTEGLGDVEKADNKNDIQIFALALLLSSTFVYNTVNKIDQGAIDLLHNVTELTDLLKARNSPDLDRVEDPADSASFFPDLVWTLRDFCLGLEIDGQLVTPDEYLENSLRPKQGSDQRVQNFNLPRLCIQKFFPKKKCFIFDLPAHQKKLAQLETLPDDELEPEFVQQVTEFCSYIFSHSMTKTLPGGIMVNGSRLKNLVLTYVNAISSGDLPCIENAVLALAQRENSAAVQKAIAHYDQQMGQKVQLPMETLQELLDLHRTSEREAIEVFMKNSFKDVDQSFQKELETLLDAKQNDICKRNLEASSDYCSALLKDIFGPLEEAVKQGIYSKPGGHNLFIQKTEELKAKYYREPRKGIQAEEVLQKYLKSKESVSHAILQTDQALTETEKKKKEAQVKAEAEKAEAQRLAAIQRQNEQMMQERERLHQEQVRQMEIAKQNWLAEQQKMQEQQMQEQAAQLSTTFQAQNRSLLSELQHAQRTVNNDDPCVLL

SEQ ID NO: 16 - DNA, 498 bp, Homo sapiens
  CDS (1)..(498)
atggagctctgccgatccctggccctgctggggggctccctgggcctgatgttctgcctgattgctttgagcaccgatttctggtttgaggctgtgggtcccacccactcagctcactcgggcctctggccaacagggcatggagacatcatatcaggctacatccacgtgacgcagaccttcagcattatggctgttctgtgggccctggtgtccgtgagcttcctggtcctgtcctgcttcccctcactgttccccccaggccacggcccgcttgtctcaaccaccgcagcctttgctgcagccatctccatggtggtggccatggcggtgtacaccagcgagcggtgggaccagcctccacacccccagatccagaccttcttctcctggtccttctacctgggctgggtctcagctatcctcttgctctgtacaggtgccctgagcctgggtgctcactgtggcggtccccgtcctggctatgaaaccttgtga

SEQ ID NO: 17 - protein, 165 aa, Homo sapiens
MELCRSLALLGGSLGLMFCLIALSTDFWFEAVGPTHSAHSGLWPTGHGDIISGYIHVTQTFSIMAVLWALVSVSFLVLSCFPSLFPPGHGPLVSTTAAFAAAISMVVAMAVYTSERWDQPPHPQIQTFFSWSFYLGWVSAILLLCTGALSLGAHCGGPRPGYETL

SEQ ID NO: 18 - DNA, 2058 bp, Homo sapiens
  CDS (242)..(1483)
gcacgaggaagccacagatctcttaagaactttctgtctccaaaccgtggctgctcgataaatcagacagaacagttaatcctcaatttaagcctgatctaacccctagaaacagatatagaacaatggaagtgacaacaagattgacatggaatgatgaaaatcatctgcgcaactgcttggaaatgtttctttgagtcttctctataagtctagtgttcatggaggtagcattgaagatatggttgaaagatgcagccgtcagggatgtactataacaatggcttacattgattacaatatgattgtagcctttatgcttggaaattatattaatttacgtgaaagttctacagagccaaatgattccctatggttttcacttcaaaagaaaaatgacaccactgaaatagaaactttactcttaaatacagcaccaaaaattattgatgagcaactggtgtgtcgtttatcgaaaacggatattttcattatatgtcgagataataaaatttatctagataaaatgataacaagaaacttgaaactaaggttttatggccaccgtcagtatttggaatgtgaagtttttcgagttgaaggaattaaggataacctagacgacataaagaggataattaaagccagagagcacagaaataggcttctagcagacatcagagactataggccctatgcagacttggtttcagaaattcgtattcttttggtgggtccagttgggtctggaaagtccagttttttcaattcagtcaagtctatttttcatggccatgtgactggccaagccgtagtggggtctgataccaccagcataaccgagcggtataggatatattctgttaaagatggaaaaaatggaaaatctctgccatttatgttgtgtgacactatggggctagatggggcagaaggagcaggactgtgcatggatgacattccccacatcttaaaaggttgtatgccagacagatatcagtttaattcccgtaaaccaattacacctgagcattctacttttatcacctctccatctctgaaggacaggattcactgtgtggcttatgtcttagacatcaactctattgacaatctctactctaaaatgttggcaaaagtgaagcaagttcacaaagaagtattaaactgtggtatagcatatgtggccttgcttactaaagtggatgattgcagtgaggttcttcaagacaactttttaaacatgagtagatctatgacttctcaaagccgggtcatgaatgtccataaaatgctaggcattcctatttccaatattttgatggttggaaattatgcttcagatttggaactggaccccatgaaggatattctcatcctctctgcactgaggcagatgctgcgggctgcagatgattttttagaagatttgcctcttgaggaaactggtgcaattgagagagcgttacagccctgcatttgagataagttgccttgattctgacatttggcccagcctgtactggtgtgccgcaatgagagtcaatctctattgacagcctgcttcagattttgcttttgttcgttttgccttctgtccttggaacagtcatatctcaagttcaaaggccaaaacctgagaagcggtgggctaagataggtcctactgcaaaccacccctccatatttccgtaccatttacaattcagtttctgtgacatctttttaaaccactggaggaaaaatgagatattctctaatttattcttctataacactctatatagagctatgtgagtactaatcacattgaataatagttataaaattattgtatagacatctgcttcttaaacagattgtgagttctttgagaaacagcgtggattttacttatctgtgtattcacagagcttagcacagtgcctggtaatgagcaagcatacttgccattacttttccttcccactctctccaacatcacattcactttaaatttttctgtatatagaaaggaaaactagcctgggcaacatgatgaaaccccatctccactgc

SEQ ID NO: 19 - protein, 413 aa, Homo sapiens
MVERCSRQGCTITMAYIDYNMIVAFMLGNYINLRESSTEPNDSLWFSLQKKNDTTEIETLLLNTAPKIIDEQLVCRLSKTDIFIICRDNKIYLDKMITRNLKLRFYGHRQYLECEVFRVEGIKDNLDDIKRIIKAREHRNRLLADIRDYRPYADLVSEIRILLVGPVGSGKSSFFNSVKSIFHGHVTGQAVVGSDTTSITERYRIYSVKDGKNGKSLPFMLCDTMGLDGAEGAGLCMDDIPHILKGCMPDRYQFNSRKPITPEHSTFITSPSLKDRIHCVAYVLDINSIDNLYSKMLAKVKQVHKEVLNCGIAYVALLTKVDDCSEVLQDNFLNMSRSMTSQSRVMNVHKMLGIPISNILMVGNYASDLELDPMKDILILSALRQMLRAADDFLEDLPLEETGAIERALQPCI

SEQ ID NO: 20 - DNA, 1216 bp, Homo sapiens
  CDS (91)..(420)
ttcggcacttgggagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccagggagaaagaacttccccatacaaataaggcatgaggactatgtgaaaaataaccttgcaggagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtgggggcggggccggggggactctggtatctaattctttaatgattcctataaatctaatgacattcaataaagttgagcaaacattttactt

SEQ ID NO: 21 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 22 - DNA, 1140 bp, Homo sapiens
  CDS (27)..(302)
cctccgacagcctctccacaggtaccatgaaggtctccgcggcacgcctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctgaccacgacgagcagcagttacaaaaaccttccccaggctggacgtggtggctcagccttgtaatcccagcactttgggaggccaaggtgggtggatcacttgaggtcaggagttcgagacagcctggccaacatgatgaaaccccatgtgtactaaaaatacaaaaaattagccgggcgtggtagcgggcgcctgtagtcccagctactcgggaggctgaggcaggagaatggcgtgaacccgggagcggagcttgcagtgagccgagatcgcgccactgcactccagcctgggcgacagagcgagactccgtctcaaaaaaaaaaaaaaaaaaaaaaaaaatacaaaaattagccgcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgcaagggctctgtgacaaggaaggaagtcagcatgcctctagaggcaaggaagggaggaacactgcactcttaagcttccgccgtctcaacccctcacaggagcttactg

SEQ ID NO: 23 - protein, 91 aa, Homo sapiens
MKVSAARLAVILIATALCAPASASPYSSDTTPCCFAYIARPLPRAHIKEYFYTSGKCSNPAVVFVTRKNRQVCANPEKKWVREYINSLEMS

SEQ ID NO: 24 - DNA, 1860 bp, Homo sapiens
  CDS (640)..(1851)
tgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaactcttcagacactgagggggcaccagaggagcagactacaagaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtgagaagtacaactgagaaatcccttttgaaggaaggttaatgtaaccca

SEQ ID NO: 25 - protein, 403 aa, Homo sapiens [sequence omitted]

SEQ ID NO: 26 - DNA, 6746 bp, Homo sapiens
  CDS (2210)..(2261)
  CDS (3111)..(3214)
  CDS (3749)..(3847)
  CDS (5296)..(5467)
  CDS (6353)..(6360)
ctgcagtgtttggtctcaccaagtttcccacaataaagagacatgagtcacctttcaagaccctttacccccaagaatgtggtcttcacacatgagaccaaggtctacaagtggtcaggagagagggggtctgctcagatgggggagtagtgcctgagctggcctcaagagggttaagtggccctgcactgaaaacctggacactgagttagggtagggctgggggaaaacttgggctttggagtcgtagggtctgggttcaaatccacagaccattcccttcctagctgtgtgttggtgggtaattcactggatctttctgagtcctggtttcctcatctgaggtaaaacgagtttgccggttggtctgagagctgttctaggcatggtggggagaccctgacaggcagaggcagccctgctctcaagcagttgatttacagctggggaaacaagacagccacaaatgcaatacctcaaactcaacttctcaccagaaagctccttttcctaattttcacagccagtccctcagcctcctgggccccaaatactagtaaaacctttgcctcctctctcttctttctttcttgtaatcatataggtacaaagtcctaccaattcttcctgaaatatgtttccttatcaaaaagtcctgcaaagccgtgcgtggttgctcatgcctataatcccagcactttggaggctgggaggatcgcttgagtccaggagttcgagaccagcctggacaacatatggagacccatctctaccaaaaattttaaaatcagcaggggtggtagtggcaagcacctgtggtctcatctacttgggaggctgaggtggggggattgttggagcctgggcggttgaggctgcagtgatctgtgattgcaccactgcactctagcctgagggacagagcaagaacttgtatcagaaaaaaaaaaaaaaagtcctgcggtactggacactgccattgcctatacgattcccactccctcatcctccctagcaggatatcaattttgttcgaagtgtcaatgaaggccaggtgcggtggctgatgcctgtaatcctaacactttgggaggccgaggcaggcggatcacctgaggtcaggagttcaagaccagcctggccaacatggtgaaaccctgtctctactaaaaacacacaaattagcagggcatggtggcgtgcacctgtaatcccagctactcaggaggctgagacaggagaatcacttgaacccggagtggaggttgcaatcagccaagatcacaccactgcacttcagcttgggtgacaagagtgaaactctgtctcaaaaaagaaaaacaaaacaaaaacaaacaacaacaacaaaaagcaaagtgtcagtgaaggtccagcaaaagactcccttcctattgccctttgcagccagggtcatcatgtgacacagttcagatcaatgagatggaggctgagggtccctgggaaagatgtttttcctatacaggtaccacctctttcagcttcactctttccattttccacgtgaacaggccttgtagcctggaggagctacagctgcctttttgagatgctgaggcaccctgtctgaagaaggccctcacatcactcaacttgactactgggtgagcccttggagaggcttcccagcctctgctcttcaagccgaagtaccacaggggacacgagtcccagagttacaggaccccagctatggttcatgtgtaaagggaaccattaggcaaccaggggaaatgatgaagaagatctacatttacaaatgtggaaagatgttcgtggtatattgttaaattaaaaagctgtttaaaaatagtttttgggtcaagtgagatgactcacttatacttttagtataagtatgtcccatgcaatatctggaacgtacttgtactaaggggtttctccctccatcggcacatcccaggcatcctggcagctgctggcctccagcaaccccacattctagttgtgtgggagtgggttgtggcatggaccctgtgggctaccactgccctgacctgcttcttcacacactggtatttgtatctgtggtaaacccagtgacacgggggagatgacatacaaaaagggcaggacctgagaaagattaagctgcaggctccctgcccataaaacagggtgtgaaaggcatctcagcggctgccccaccatggctacctgggccctcctgctccttgcagccatgctcctgggcaacccaggtaaggccttcccctcgggatcgatcctgatggcccacccagcctcgcactctcaggctggctgaacctggagcttggactctgtgggcacccaggtgcccctgcctccccccggccttctcccccgtcatggaggcctggccctcccctcagagccaggcttagtccggtgtgctgcccagcctgtcactggcctggccaaggaggagagacaggccagggattctggtcctaactctactggccacactgtgtggcctgagacccccctttccctcccaagcccctgcctccgcatctgcgtggtgaaggccattggcctcatcggtggatctgcgtttcctcgggcctacactgtctaggattgtgcggggctggtgagagaacaagatctcttccgtgttcaaggcagacttcctgccccctgcaccctgctctctcccaggccttgaggtcagtgtgagccccaagggcaagaacacttctggaagggagagtggatttggctgggccatctggatggaaggtaaaaaaagaaaatcccttgaaaggagattgagggaagtttctagacaaaccgacccccaaatctgtgttgctgggggaacagaggagaagagagagtctcgccctcctggctttctagaaggaacgtgagaacacgtgtttgtgctgagagtgggtcagagcggctccagggcaaagcatgtggacaggtatcctggccccctgcaaggcccagctcctgtcctaggccctggtcacctcctggactcccaccagccaggagaacgggctttccctctccttccgcctgcggaggggaagctgaagtctggtcttcctcaggtctggtcttctctcgtctgagccctgagtactacgacctggcaagagcccacctgcgtgatgaggagaaatcctgcccgtgcctggcccaggagggcccccaggtacgtgttggctctctgctcacctgccacagtccctctcctttccctcctccctggtggctcctggggtgaggtctggagctctctaatggtcaggaggtgggagtggaggctgggctgtttctgacgatgctggttttgttgaattcatgtctggccaggagggctacaggtatctggcagactcctccaggaggatcctctggggtctcaccctccaaggagcctggggctgcagaacccaaataggcagactcccctgggagttcctcaataggagaggggcaagtgcagggctgggaaagtactggggttgtgggaggctgtttctggggtgtctcagagcctctaagacaagcaaaagggtgggtaggggccaggcagccagttcaggccttcagtgtatccacgctctgggaagagatcacggacattcctgccggcctcagaaacacaaagggcccctttcctgggcactttcacgcgctcccagagtgtctgagagaccatcataagggctttctttcctgacagggtgacctgttgaccaaaacacaggagctgggccgtgactacaggacctgtctgacgatagtccaaaaactgaagaagatggtggataagcccacccaggtgaggccaaggggctacagagcctcctgtctgctgctcaatggaggggccagcctgtgaccaggtcggggatcggggagcccgggggcaccttgcacagtgatcctgggggagggcttcctagaagggaatctgtgagtccccgtgtgtctgtggatgaatttcagagaacttgtgaaattgtgactctctggaactgtgtaagtcagacggcagagtatacatggttttcatcatgtatcctcaaagagggcttgtcccagagaagttaggaatcttcccctaaagccctaacatttgtgtccaaggcagagtttgagaagctagttccccaagaggcctgggtcaggactgataaatcccagatctgctacttccaagctgcatggccttgggcaagtcacttccactttctgagcccctgttatcttatctttgaaatgtgatggataatagtccctatcttgcaagttgtcaaacccttttttttttttttccttgagataggatcttactctgagacccaggctggagtgcactggtgtgatcttggctcactgcaacctctgcctccctggcccaagcaattctcctgtctaagcctcctgagtacctggggctccaggtgtgcgccaccatgcccagctaatttttgtacttttgtagaaacagggtctcactgtgttgcccaggctggtctccaacttctgagctgaagcaatccacctgccttggcctcccaaagtgtgggattataggcatgagccactgcacctggctgctgaagctttttaaaagagctgagggctgggatgtgcttagctccacgtccagcactgagtaaatgcttaacgaatgactgtgttactaccaagaattattgtttcactctccctccttccctctcctctgctgccccaaactactcagcatcctggcactgcaggctcgcacttagccctggatacccagattcatcctcctcccctgggatggcatagaagagactttaaaaccaaatgagccaagactccaagctctgaccacacctcccacccccaccagtcttctctatgcaccccctctatatctggagcccccagccaggttctggaccaaggtagctacatggcagagcatttaatgtgtgcctggcagccatgggcaccattctccacacagaaggcagggacaggtgcacaaggggctgagaccccagcagggctaactgtccttgtctcaggagccctacctggccagtcttgggccaggccttggggactgggagtaggggctgacccgtctgtacagtctctggccccatggcaccaggtgccagctcctcgcacccagtactcccattgctagggctgctggaacctgcagggttggcagagctgggcaggactcaccctataaccatgtccactgtggtgctgctgctgcagagaagtgtttccaatgctgcgacccgggtgtgtaggacggggaggtcacgatggcgcgacgtctgcagaaatttcatgaggaggtatcagtctagagttatccagggcctcgtggccggagaaactgcccagcagatctgtgaggacctcaggttgtgtataccttctacaggtgagtgcagaggtgacagcagggatacctcctgagggttggagacagcttcccccaggatatatcaaagctgcctccttactcccccatctcccagcatgggaaagtgtggagaattgagcagatggactttagctagaaatgtttgagaaatactgattagagcttgggcttcagacacaggtggttgtggagtaaaatctggtctccatctctccctggctgtgtgaccttaagcaaataacttgacctctctgagcttcagtttcttcatctgtgaaggagagatagcaatcctgatttttgagattggaatgagaattgaaggaggtcaccgtgtgtgtggacctgaccctggggaaatgtcctcagactgaggctattcaaggtcatcagaccctcagtcaaactccaacccagcccagcacatggcccctggggtcgggagctggggccatatcctcccccacaatcctgggccctgagatctgggctagggaacccttcaggcaggggagcatgaggcctttccctccatggctgcccaggctgtgctgagagaacagatctcggctgtaggaaacggggccagaaaggggcctcggtgattggctctggcagctcagctggcacttgccaatagctctgggattttatgctggcagatcgggggtccccaccatttcctgtcattggagcttgtggcttttctattcaaggccccacagcctgctcaggctgccgactggcttccaggatgtgcctctgggtgtgttcagtagggtcaggtggctctgggaccttaagcaagtaacattctgagtgcctgcttctccttgaggacccaccacatctgcccacagctagctgttctctccgctccaggtcccctctgagccctctcaccttgtcctgtggaagaagcacaggctcctgtcctcagatcccgggaacgtcagcaacctctgccggctcctcgcttcctcgatccagaatccactctccagtctccctcccctgactccctctgctgtcctcccctctcaggagaataaagtgtcaagcaagattttagccgcagctgcttcttctttggtggatttgaggggtgggtgtcagtggcatgctggggtgagctgtgtagtccttcaataaatgtctgtcgtgtgtcccatacactgttgtagatgttatggatttagtggtgaacgagacaaccttaacagcattcacacagttagtcgtgaaatgcttactgagcactcaccacagccatgca

SEQ ID NO: 27 - protein, 145 aa, Homo sapiens
MATWALLLLAAMLLGNPGLVFSRLSPEYYDLARAHLRDEEKSCPCLAQEGPQGDLLTKTQELGRDYRTCLTIVQKLKKMVDKPTQRSVSNAATRVCRTGRSRWRDVCRNFMRRYQSRVIQGLVAGETAQQICEDLRLCIPSTGPL

SEQ ID NO: 28 - DNA, 3655 bp, Homo sapiens
  CDS (358)..(1416)
cttcagatagattatatctggagtgaaggatcctgccacctacgtatctggcatagtattctgtgtagtgggatgagcagagaacaaaaacaaaataatccagtgagaaaagcccgtaaataaaccttcagaccagagatctattctccagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttggtttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatataggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctttctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttcta

SEQ ID NO: 29 - protein, 352 aa, Homo sapiens [sequence omitted]

SEQ ID NO: 30 - DNA, 983 bp, Homo sapiens
  CDS (10)..(552)
gtggaattcatggcatctacttcgtatgactattgcagagtgcccatggaagacggggataagcgctgtaagcttctgctggggataggaattctggtgctcctgatcatcgtgattctgggggtgcccttgattatcttcaccatcaaggccaacagcgaggcctgccgggacggccttcgggcagtgatggagtgtcgcaatgtcacccatctcctgcaacaagagctgaccgaggcccagaagggctttcaggatgtggaggcccaggccgccacctgcaaccacactgtgatggccctaatggcttccctggatgcagagaaggcccaaggacaaaagaaagtggaggagcttgagggagagatcactacattaaaccataagcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtgagatcccaggaagctggcacatcttggaaggtccgtcctgctcggcttttcgcttgaacattcccttgatctcatcagttctgagcgggtcatggggcaacacggttagcggggagagcacggggtagccggagaagggcctctggagcaggtctggaggggccatggggcagtcctgggtgtggggacacagtcgggttgacccagggctgtctccctccagagcctccctccggacaatgagtcccccctcttgtctcccaccctgagattgggcatggggtgcggtgtggggggcatgtgctgcctgttgttatgggttttttttgcggggggggttgcttttttctggggtctttgagctccaaaaaataaacacttcctttgagggagagcaaaaaaaaaaaaaaaaaaaaaaaaaaaaaaaaaaaa

SEQ ID NO: 31 - protein, 180 aa, Homo sapiens
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 32 - DNA, 768 bp, Homo sapiens
  CDS (62)..(556)
ccttcagcataaaagctgatccacaaacaagaggagcaccagacctcctcttggcttcgagatggcttcgccacaccaagagcccaaacctggagacctgattgagattttccgccttggctatgagcactgggccctgtatataggagatggctacgtgatccatctggctcctccaagtgagtaccccggggctggctcctccagtgtcttctcagtcctgagcaacagtgcagaggtgaaacgggggcgcctggaagatgtggtgggaggctgttgctatcgggtcaacaacagcttggaccatgagtaccaaccacggcccgtggaggtgatcatcagttctgcgaaggagatggttggtcagaagatgaagtacagtattgtgagcaggaactgtgagcactttgtcgcccagctgagatatggcaagtcccgctgtaaacaggtggaaaaggccaaggttgaagtcggtgtggccacggcgcttggaatcctggttgttgctggatgctcttttgcgattaggagataccaaaaaaaagcaacagcctgaagcagccacaaaatcctgtgttagaagcagctgtgggggtcccagtggagatgagcctcccccatgcctccagcagcctgaccctcgtgccctgtctcaggcgttctctagatcctttcctctgtttccctctctcgctggcaaaagtatgatctaattgaaacaagactgaaggatcaataaacagccatctgccccttcaaaaaaaaaaa

SEQ ID NO: 33 - protein, 164 aa, Homo sapiens
MASPHQEPKPGDLIEIFRLGYEHWALYIGDGYVIHLAPPSEYPGAGSSSVFSVLSNSAEVKRGRLEDVVGGCCYRVNNSLDHEYQPRPVEVIISSAKEMVGQKMKYSIVSRNCEHFVAQLRYGKSRCKQVEKAKVEVGVATALGILVVAGCSFAIRRYQKKATA

SEQ ID NO: 34 - DNA, 714 bp, Homo sapiens
  misc_feature (394)..(394): n is a, c, g, or t
ttagttttaaattatatatatttttttctttttgaaattttttttcaaaattccatagttatatacagtaacatcaatttcaacaacagcagtacaaagcacacatttgacttacaggagcactgattgccaaagaataccaaagtgtttcaccttgatgatccatgggaggaccccacaacactacagacaggtaggagggagaacaatgacattttgccgtgaacttagttgagctcacaggaaaacaaaaacaaaaaacacatcaccaggattttgcattttacattcactaggatgtatgacagatagagttaaaacatcaacaacttaaatataattaaatactttttaagaaatattatgctttaaattatttaaaaataattcttgnggggatgttataataaaattaatgtcacactgactggggggaagttggggggaaaaataataaagcttttctttaagctctgatcctcagtccatgtattgaagggctcatggcttcaaattttggaaatttttggtccagatggcttattggaatccctggacccatcttcctttgggggcaacacagaaccttcttgcccggttttgtctttggaagattacagctttcttgggacattggaggttgggtgaattctttgtggtacctttccagcctttggataaggaaatttccttatggataccgttaaagccgca

SEQ ID NO: 35 - DNA, 878 bp, Homo sapiens
  CDS (39)..(827)
cagattttcaggttgattgatgtgggacagcagccacaatgaggaactcctatagatttctggcatcctctctctcagttgtcgtttctctcctgctaattcctgaagatgtctgtgaaaaaattattggaggaaatgaagtaactcctcattcaagaccctacatggtcctacttagtcttgacagaaaaaccatctgtgctggggctttgattgcaaaagactgggtgttgactgcagctcactgtaacttgaacaaaaggtcccaggtcattcttggggctcactcaataaccagggaagagccaacaaaacagataatgcttgttaagaaagagtttccctatccatgctatgacccagccacacgcgaaggtgaccttaaacttttacagctgacggaaaaagcaaaaattaacaaatatgtgactatccttcatctacctaaaaagggggatgatgtgaaaccaggaaccatgtgccaagttgcagggtgggggaggactcacaatagtgcatcttggtccgatactctgagagaagtcaatatcaccatcatagacagaaaagtctgcaatgatcgaaatcactataattttaaccctgtgattggaatgaatatggtttgtgctggaagcctccgaggtggaagagactcgtgcaatggagattctggaagccctttgttgtgcgagggtgttttccgaggggtcacttcctttggccttgaaaataaatgcggagaccctcgtgggcctggtgtctatattcttctctcaaagaaacacctcaactggataattatgactatcaagggagcagtttaaataaccgtttcctttcatttactgtggcttcttaatcttttcacaaataaa

SEQ ID NO: 36 - protein, 262 aa, Homo sapiens
MRNSYRFLASSLSVVVSLLLIPEDVCEKIIGGNEVTPHSRPYMVLLSLDRKTICAGALIAKDWVLTAAHCNLNKRSQVILGAHSITREEPTKQIMLVKKEFPYPCYDPATREGDLKLLQLTEKAKINKYVTILHLPKKGDDVKPGTMCQVAGWGRTHNSASWSDTLREVNITIIDRKVCNDRNHYNFNPVIGMNMVCAGSLRGGRDSCNGDSGSPLLCEGVFRGVTSFGLENKCGDPRGPGVYILLSKKHLNWIIMTIKGAV

SEQ ID NO: 37 - DNA, 1642 bp, Homo sapiens
  CDS (65)..(1501)
ccagatctcagaggagcctggctaagcaaaaccctgcagaacggctgcctaatttacagcaaccatgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaagaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttctactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctataaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaatatt

SEQ ID NO: 38 - protein, 478 aa, Homo sapiens
MSTNGDDHQVKDSLEQLRCHFTWELSIDDDEMPDLENRVLDQIEFLDTKYSVGIHNLLAYVKHLKGQNEEALKSLKEAENLMQEEHDNQANVRSLVTWGNFAWMYYHMGRLAEAQTYLDKVENICKKLSNPFRYRMECPEIDCEEGWALLKCGGKNYERAKACFEKVLEVDPENPESSAGYAISAYRLDGFKLATKNHKPFSLLPLRQAVRLNPDNGYIKVLLALKLQDEGQEAEGEKYIEEALANMSSQTYVFRYAAKFYRRKGSVDKALELLKKALQETPTSVLLHHQIGLCYKAQMIQIKEATKGQPRGQNREKLDKMIRSAIFHFESAVEKKPTFEVAHLDLARMYIEAGNHRKAEENFQKLLCMKPVVEETMQDIHFYYGRFQEFQKKSDVNAIIHYLKAIKIEQASLTRDKSINSLKKLVLRKLRRKALDLESLSLLGFVYKLEGNMNEALEYYERALRLAADFENSVRQGP

SEQ ID NO: 39 - DNA, 20 bp, Artificial Sequence
  Artifically synthesized oligonucleotide primer
atggcatcagagatccacat

SEQ ID NO: 40 - DNA, 20 bp, Artificial Sequence
  Artifically synthesized oligonucleotide primer
gcttatggtacatgcctttc

SEQ ID NO: 41 - DNA, 20 bp, Artificial Sequence
  Artifically synthesized oligonucleotide primer
aaggatggaccacacagagg

SEQ ID NO: 42 - DNA, 20 bp, Artificial Sequence
  Artifically synthesized oligonucleotide primer
tggaagatgggaaaggtgag

SEQ ID NO: 43 - DNA, 21 bp, Artificial Sequence
  Artifically synthesized oligonucleotide primer
tcatcttgctggttctgattg

SEQ ID NO: 44 - DNA, 21 bp, Artificial Sequence
  Artifically synthesized oligonucleotide primer
acgagaacgttgagattttcg

SEQ ID NO: 45 - DNA, 22 bp, Artificial Sequence
  Artifically synthesized oligonucleotide primer
gctatagccttggctgtgatat

SEQ ID NO: 46 - DNA, 22 bp, Artificial Sequence
  Artifically synthesized oligonucleotide primer
gccttgcttgcttcgatttggg

SEQ ID NO: 47 - DNA, 22 bp, Artificial Sequence
  Artifically synthesized oligonucleotide primer
gcaaatgcaagaacgggacact

SEQ ID NO: 48 - DNA, 24 bp, Artificial Sequence
  Artifically synthesized oligonucleotide primer
tcagggagaccagagctttcacac

SEQ ID NO: 49 - DNA, 20 bp, Artificial Sequence
  Artifically synthesized oligonucleotide primer
atttattttccccaacctcc

SEQ ID NO: 50 - DNA, 20 bp, Artificial Sequence
  Artifically synthesized oligonucleotide primer
acaatgacattttgccgtga

SEQ ID NO: 51 - DNA, 20 bp, Artificial Sequence
  Artifically synthesized oligonucleotide primer
tacagctggctcctgaagga

SEQ ID NO: 52 - DNA, 20 bp, Artificial Sequence
  Artifically synthesized oligonucleotide primer
cggctaacggataagcagag

SEQ ID NO: 53 - DNA, 20 bp, Artificial Sequence
  Artifically synthesized oligonucleotide primer
ttaaatgataatcccagccc

SEQ ID NO: 54 - DNA, 20 bp, Artificial Sequence
  Artifically synthesized oligonucleotide primer
aagattactggcctcgctga

SEQ ID NO: 55 - DNA, 21 bp, Artificial Sequence
  Artifically synthesized oligonucleotide primer
accctacatggtcctacttag

SEQ ID NO: 56 - DNA, 20 bp, Artificial Sequence
  Artifically synthesized oligonucleotide primer
aagtgacccctcggaaaaca

SEQ ID NO: 57 - DNA, 20 bp, Artificial Sequence
  Artifically synthesized oligonucleotide primer
agttcctgccaggcctttac

SEQ ID NO: 58 - DNA, 20 bp, Artificial Sequence
  Artifically synthesized oligonucleotide primer
cagcagaaagaggaggctgt

SEQ ID NO: 59 - DNA, 20 bp, Artificial Sequence
  Artifically synthesized oligonucleotide primer
agccacatcgctcagaacac

SEQ ID NO: 60 - DNA, 21 bp, Artificial Sequence
  Artifically synthesized oligonucleotide primer
gaggcattgctgatgatcttg